(19) United States
(12) Patent Application Publication
(10) Pub. No.: US 2022/0175760 A1
(43) Pub. Date: Jun. 9, 2022

(54) PHARMACEUTICAL FORMULATIONS

(71) Applicant: Janssen Pharmaceutica NV, Beerse (BE)

(72) Inventors: Kristof Leonard KIMPE, Beerse (BE); Sanket Manoj SHAH, Antwepen (BE); Audrey Antoinette Renee LATHUILE, Eindhoven (NL); Rene HOLM, Woluwe Saint Pierre (BE); Thomas Eddy R NEEFS, Herentals (BE); Hana PROKOPCOVA, Gierle (BE)

(21) Appl. No.: 17/431,448

(22) PCT Filed: Feb. 20, 2020
(86) PCT No.: PCT/EP2020/054488

§ 371 Date: Aug. 17, 2021

(30) Foreign Application Priority Data
Feb. 22, 2019 (CN) PCT/CN2019/075844

(51) Int. Cl. A61K 31/4725; A61K 9/48; A61K 47/32; A61K 47/22; A61K 47/10

(52) U.S. Cl. CPC A61K 31/4725; A61K 9/4833; A61K 47/32; A61K 47/10; A61K 47/22; A61K 9/4816; A61K 9/4866

(57) ABSTRACT

The invention relates to pharmaceutical formulations comprising an active pharmaceutical ingredient, a polyethylene glycol having a freezing point of at least about 30° C., and a crystallisation rate inhibitor. Solid dosage forms comprising said pharmaceutical formulations, processes for preparing these and their use in methods of treatment are also described.

### FIELD OF THE INVENTION

The present invention relates to pharmaceutical formulations comprising an active pharmaceutical ingredient, a polyethylene glycol having a freezing point of at least about 30° C., and a crystallisation rate inhibitor, and solid dosage forms comprising said pharmaceutical formulations. The invention also relates to processes to prepare such pharmaceutical formulations and to the use of such pharmaceutical formulations for the treatment of a disease, syndrome, condition, or disorder.

### BACKGROUND OF THE INVENTION

Many active pharmaceutical ingredients (API) have properties such as hydrophobicity and instability leading to challenges in providing suitable pharmaceutical formulations.

MALT1 (mucosa-associated lymphoid tissue lymphoma translocation 1) is a key mediator of the classical NFKB signaling pathway. WO 2018/119036 discloses a class of active pharmaceutical agents which are MALT1 inhibitors that may provide a therapeutic benefit to patients suffering from cancer and/or immunological diseases.

There exists a need for improved pharmaceutical formulations of active pharmaceutical ingredients, such as the MALT1 inhibitors described in WO 2018/119036. In particular there exists a need for pharmaceutical formulations with an acceptable bioavailability, in particular in a solid dosage form.

### SUMMARY OF THE INVENTION

The present invention is directed to a pharmaceutical formulation, comprising:
a) a polyethylene glycol having a freezing point of at least about 30° C.;
b) an active pharmaceutical ingredient that is soluble in molten polyethylene glycol wherein the polyethylene glycol is as defined in a); and
c) a crystallisation rate inhibitor.

Embodiments of the invention include a pharmaceutical formulation as described herein, wherein the active pharmaceutical ingredient is a MALT1 inhibitor.

The invention also provides a solid dosage form comprising a pharmaceutical formulation described herein.

In embodiments in which the active pharmaceutical ingredient is a MALT1 inhibitor, the invention provides methods for treating or ameliorating a disease, syndrome, condition, or disorder in a subject, including a mammal and/or human in which the disease, syndrome, condition, or disorder is affected by the inhibition of MALT1, including but not limited to, cancer and/or immunological diseases, using pharmaceutical formulations and solid dosage forms described herein.

The present invention is also directed to the use of such pharmaceutical formulations in the preparation of a medicament wherein the medicament is prepared for treating a disease, syndrome, disorder or condition that is affected by the inhibition of MALT1, such as cancer and/or immunological diseases.

Exemplifying the invention are methods of treating a disease, syndrome, condition, or disorder mediated by MALT1, selected from the group consisting of lymphomas, leukemias, carcinomas, and sarcomas, e.g. non-Hodgkin's lymphoma (NHL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, and GIST (gastrointestinal stromal tumor), comprising, consisting of, and/or consisting essentially of, administering to a subject in need thereof a therapeutically effective amount of the pharmaceutical formulation or solid dosage form.

In another embodiment, the present invention is directed to pharmaceutical formulations and solid dosage forms described herein for use in the treatment of a disease, syndrome, condition, or disorder affected by the inhibition of MALT1, such as cancer and/or immunological disease. The disease, syndrome, condition, or disorder may be selected from the group consisting of lymphomas, leukemias, carcinomas, and sarcomas, e.g. non-Hodgkin's lymphoma (NHL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, and GIST (gastrointestinal stromal tumor).

The invention also provides a process for preparing a pharmaceutical formulation described herein, the process comprising the steps of:
a) forming a melt comprising polyethylene glycol having a freezing point of at least about 30° C., an active pharmaceutical ingredient, and a crystallisation rate inhibitor, wherein the forming a melt step comprises heating polyethylene glycol to a temperature above its freezing point; and
b) cooling the melt below the freezing point of the polyethylene glycol;
   to provide a pharmaceutical formulation described herein.

The invention also provides a process for preparing a solid dosage form described herein, the process comprising the steps of:
a) forming a melt comprising polyethylene glycol having a freezing point of at least about 30° C., an active pharmaceutical ingredient, and a crystallisation rate inhibitor, wherein the forming a melt step comprises heating polyethylene glycol to a temperature above its freezing point;
b) filling a hard capsule with the melt; and
c) cooling the filled capsule below the freezing point of the polyethylene glycol;
   to provide a solid dosage form described herein.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific disclosure of the drawings. In the drawings:

FIG. 1 is an X-ray powder diffraction (XRPD) pattern of the crystalline form of Compound A monohydrate as obtained in Example 1.

FIG. 2 is an XRPD of the precipitate formed when a supersaturated solution of Compound A in NMP is added to Fasted state simulated intestinal fluid (FaSSIF) containing 1% PVPVA64.

### DETAILED DESCRIPTION OF THE INVENTION

The disclosure may be more fully appreciated by reference to the following description, including the following glossary of terms and the concluding examples. It is to be appreciated that certain features of the disclosed pharmaceutical formulations and methods which are, for clarity, described herein in the context of separate aspects, may also be provided in combination in a single aspect. Conversely, various features of the disclosed pharmaceutical formulations and methods that are, for brevity, described in the context of a single aspect, may also be provided separately or in any sub-combination.

Some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

Throughout the description and claims of this specification, the words “comprise” and “contain” and variations of the words, for example “comprising” and “comprises”, mean “including but not limited to”, and are not intended to (and do not) exclude other components.

With reference to substituents, the term “independently” refers to the situation where when more than one substituent is possible, the substituents may be the same or different from each other.

The term “alkyl” whether used alone or as part of a substituent group, refers to straight and branched carbon chains having 1 to 8 carbon atoms. Therefore, designated numbers of carbon atoms (e.g., C₁₋₈) refer independently to the number of carbon atoms in an alkyl moiety or to the alkyl portion of a larger alkyl-containing substituent. In substituent groups with multiple alkyl groups such as, (C₁₋₆alkyl)₂amino-, the C₁₋₆alkyl groups of the dialkylamino may be the same or different.

The term “alkoxy” refers to an —O-alkyl group, wherein the term “alkyl” is as defined above.

The terms “alkenyl” and “alkynyl” refer to straight and branched carbon chains having 2 to 8 carbon atoms, wherein an alkenyl chain contains at least one double bond and an alkynyl chain contains at least one triple bond.

The term “cycloalkyl” refers to saturated or partially saturated, monocyclic or polycyclic hydrocarbon rings of 3 to 14 carbon atoms. Examples of such rings include cyclopropyl, cyclobutyl, cyclopentyl, cyclohexyl, cycloheptyl, and adamantyl.

The term “heterocyclyl” refers to a nonaromatic monocyclic or bicyclic ring system having 3 to 10 ring members that include at least 1 carbon atom and from 1 to 4 heteroatoms independently selected from N, O, and S. Included within the term heterocyclyl is a nonaromatic cyclic ring of 5 to 7 members in which 1 to 2 members are N, or a nonaromatic cyclic ring of 5 to 7 members in which 0, 1 or 2 members are N and up to 2 members are O or S and at least one member must be either N, O, or S; wherein, optionally, the ring contains 0 to 1 unsaturated bonds, and, optionally, when the ring is of 6 or 7 members, it contains up to 2 unsaturated bonds. The carbon atom ring members that form a heterocycle ring may be fully saturated or partially saturated. The term “heterocyclyl” also includes two 5 membered monocyclic heterocycloalkyl groups bridged to form a bicyclic ring. Such groups are not considered to be fully aromatic and are not referred to as heteroaryl groups. When a heterocycle is bicyclic, both rings of the heterocycle are non-aromatic and at least one of the rings contains a heteroatom ring member. Examples of heterocycle groups include, and are not limited to, pyrrolinyl (including 2H-pyrrole, 2-pyrrolinyl or 3-pyrrolinyl), pyrrolidinyl, imidazolinyl, imidazolidinyl, pyrazolinyl, pyrazolidinyl, piperidinyl, morpholinyl, thiomorpholinyl, and piperazinyl. Unless otherwise noted, the heterocycle is attached to its pendant group at any heteroatom or carbon atom that results in a stable structure.

The term “aryl” refers to an unsaturated, aromatic monocyclic or bicyclic ring of 6 to 10 carbon members. Examples of aryl rings include phenyl and naphthalenyl.

The term “heteroaryl” refers to an aromatic monocyclic or bicyclic aromatic ring system having 5 to 10 ring members and which contains carbon atoms and from 1 to 4 heteroatoms independently selected from the group consisting of N, O, and S. Included within the term heteroaryl are aromatic rings of 5 or 6 members wherein the ring consists of carbon atoms and has at least one heteroatom member. Suitable heteroatoms include nitrogen, oxygen, and sulfur. In the case of 5 membered rings, the heteroaryl ring preferably contains one member of nitrogen, oxygen or sulfur and, in addition, up to 3 additional nitrogens. In the case of 6 membered rings, the heteroaryl ring preferably contains from 1 to 3 nitrogen atoms. For the case wherein the 6 membered ring has 3 nitrogens, at most 2 nitrogen atoms are adjacent. Examples of heteroaryl groups include furyl, thienyl, pyrrolyl, oxazolyl, thiazolyl, imidazolyl, pyrazolyl, oxazolyl, thiazolyl, oxadiazolyl, triazolyl, thiadiazolyl, pyridinyl, pyridazinyl, pyrimidinyl, pyrazinyl, indolyl, isoindolyl, benzofuryl, benzothienyl, indazolyl, benzimidazolyl, benzothiazolyl, benzoxazolyl, benzisoxazolyl, benzothiadiazolyl, benzotriazolyl, quinolinyl, isoquinolinyl and quinazolinyl. Unless otherwise noted, the heteroaryl is attached to its pendant group at any heteroatom or carbon atom that results in a stable structure.

The term “halogen” or “halo” refers to fluorine, chlorine, bromine and iodine atoms.

The term “carboxy” refers to the group —C(═O)OH.

The term “formyl” refers to the group —C(═O)H.

The term “oxo” or “oxido” refers to the group (═O).

Whenever the term “alkyl” or “aryl” or either of their prefix roots appear in a name of a substituent (e.g., arylalkyl, alkylamino) the name is to be interpreted as including those limitations given above for “alkyl” and “aryl.” Designated numbers of carbon atoms (e.g., C₁-C₆) refer independently to the number of carbon atoms in an alkyl moiety, an aryl moiety, or in the alkyl portion of a larger substituent in which alkyl appears as its prefix root. For alkyl and alkoxy substituents, the designated number of carbon atoms includes all of the independent members included within a given range specified. For example C₁₋₆ alkyl would include methyl, ethyl, propyl, butyl, pentyl and hexyl individually as well as sub-combinations thereof (e.g., C₁₋₂, C₁₋₃, C₁₋₄, C₁₋₅, C₂₋₆, C₃₋₆, C₄₋₆, C₅₋₆, C₂₋₅, etc.).

In general, under standard nomenclature rules used throughout this disclosure, the terminal portion of the designated side chain is described first followed by the adjacent functionality toward the point of attachment. Thus, for example, a “C₁-C₆ alkylcarbonyl” substituent refers to a group of the formula:

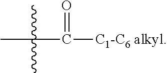

The label “R” at a stereocenter designates that the stereocenter is purely of the R-configuration as defined in the art; likewise, the label “S” means that the stereocenter is purely of the S-configuration. As used herein, the labels “*R” or “*S” at a stereocenter are used to designate that the stereocenter is of pure but unknown absolute configuration. As used herein, the label “RS” refers to a stereocenter that exists as a mixture of the R- and S-configurations.

A compound containing one stereocenter drawn without a stereo bond designation is a mixture of two enantiomers. A compound containing two stereocenters both drawn without stereo bond designations is a mixture of four diastereomers. A compound with two stereocenters both labeled “RS” and drawn with stereo bond designations is a mixture of two enantiomers with relative stereochemistry as drawn. A compound with two stereocenters both labeled “*RS” and drawn with stereo bond designations is a mixture of two enantiomers with a single, but unknown, relative stereochemistry.

Unlabeled stereocenters drawn without stereo bond designations are mixtures of the R- and S-configurations. For unlabeled stereocenters drawn with stereo bond designations, the relative and absolute stereochemistry is as depicted.

Unless otherwise noted, it is intended that the definition of any substituent or variable at a particular location in a molecule be independent of its definitions elsewhere in that molecule. It is understood that substituents and substitution patterns on the compounds of the present invention can be selected by one of ordinary skill in the art to provide compounds that are chemically stable and that can be readily synthesized by techniques known in the art as well as those methods set forth herein.

A person of ordinary skill in the art would recognize that the compounds described herein may exist as tautomers and that other tautomeric arrangements of the structures depicted herein are possible. It is understood that all tautomeric forms are encompassed by a structure where one possible tautomeric arrangement of the groups of the compound is described, even if not specifically indicated.

For example, it is understood that

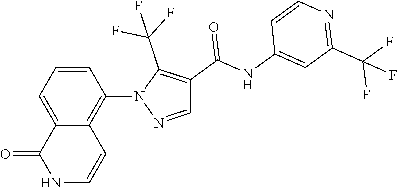 [SMILES: O=C(Nc1ccnc(C(F)(F)F)c1)c1cnn(-c2cccc3c(=O)[nH]ccc23)c1C(F)(F)F]

also encompasses by the following structure

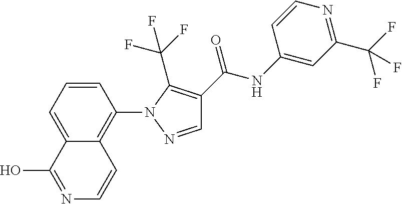 [SMILES: O=C(Nc1ccnc(C(F)(F)F)c1)c1cnn(-c2cccc3c(O)nccc23)c1C(F)(F)F]

Any convenient tautomeric arrangement may be utilized in describing the compounds.

For use in medicine, salts of compounds of Formula (I) refer to non-toxic “pharmaceutically acceptable salts.” “Pharmaceutically acceptable” may mean approved or approvable by a regulatory agency of the Federal or a state government or the corresponding agency in countries other than the United States, or that is listed in the U. S. Pharmacopoeia or other generally recognized pharmacopoeia for use in animals, and more particularly, in humans.

Other salts may, however, be useful in the preparation of compounds of Formula (I) or of their pharmaceutically acceptable salt forms thereof. Suitable pharmaceutically acceptable salts of compounds of Formula (I) include acid addition salts that can, for example, be formed by mixing a solution of the compound with a solution of a pharmaceutically acceptable acid such as, hydrochloric acid, sulfuric acid, fumaric acid, maleic acid, succinic acid, acetic acid, benzoic acid, citric acid, tartaric acid, carbonic acid or phosphoric acid. Furthermore, where the compounds of Formula (I) carry an acidic moiety, suitable pharmaceutically acceptable salts thereof may include alkali metal salts such as, sodium or potassium salts; alkaline earth metal salts such as, calcium or magnesium salts; and salts formed with suitable organic ligands such as, quaternary ammonium salts. Thus, representative pharmaceutically acceptable salts include acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide, and valerate.

Representative acids and bases that may be used in the preparation of pharmaceutically acceptable salts include acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hippuric acid, hydrobromic acid, hydrochloric acid, (+)-L-lactic acid, (+)-DL-lactic acid, lactobionic acid, maleic acid, (−)-L-malic acid, malonic acid, (+)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and bases including ammonia, L-arginine, benethamine, benzathine, calcium hydroxide, choline, deanol, diethanolamine, diethylamine, 2-(diethylamino)-ethanol, ethanolamine, ethylenediamine, N-methylglucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, sodium hydroxide, triethanolamine, tromethamine, and zinc hydroxide.

Embodiments of the present invention include prodrugs of compounds of Formula (I). In general, such prodrugs will be functional derivatives of the compounds that are readily convertible in vivo into the required compound. Thus, in the methods of treating or preventing embodiments of the present invention, the term “administering” encompasses the treatment or prevention of the various diseases, conditions, syndromes and disorders described with the compound specifically disclosed or with a compound that may not be specifically disclosed, but which converts to the specified compound in vivo after administration to a patient. Conventional procedures for the selection and preparation of suitable prodrug derivatives are described, for example, in “Design of Prodrugs”, ed. H. Bundgaard, Elsevier, 1985.

Where the compounds of Formula (I) have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the compounds may exist as polymorphs and as such are intended to be included in the present invention. In addition, some of the compounds may form solvates with water (i.e., hydrates) or common organic solvents, and such solvates are also intended to be encompassed within the scope of this invention. The skilled artisan will understand that the term compound as used herein, is meant to include solvated compounds of Formula (I).

Where the processes for the preparation of the compounds of Formula (I) give rise to mixture of stereoisomers, these isomers may be separated by conventional techniques such as, preparative chromatography. The compounds may be prepared in racemic form, or individual enantiomers may be prepared either by enantiospecific synthesis or by resolution. The compounds may, for example, be resolved into their component enantiomers by standard techniques such as, the formation of diastereomeric pairs by salt formation with an optically active acid such as, (−)-di-p-toluoyl-d-tartaric acid and/or (+)-di-p-toluoyl-l-tartaric acid followed by fractional crystallisation and regeneration of the free base. The compounds may also be resolved by formation of diastereomeric esters or amides, followed by chromatographic separation and removal of the chiral auxiliary. Alternatively, the compounds may be resolved using a chiral HPLC column.

In one embodiment of the pharmaceutical formulation of the present invention, the compound of Formula (I) is a compound comprising, consisting of, and/or consisting essentially of the (+)-enantiomer wherein said compound is substantially free from the (−)-isomer. In the present context, substantially free means less than about 25%, preferably less than about 10%, more preferably less than about 5%, even more preferably less than about 2% and even more preferably less than about 1% of the (−)-isomer calculated as

$$\% ( + ) \text{-} enantiomer = \frac{( mass ( + ) \text{-} enantionmer )}{( mass ( + ) \text{-} enantionmer ) + ( mass ( - ) \text{-} enantionmer )} \times 100.$$

In another embodiment of the pharmaceutical formulation of the present invention, the compound of Formula (I) is a compound comprising, consisting of, and consisting essentially of the (−)-enantiomer wherein said compound is substantially free from the (+)-isomer. In the present context, substantially free from means less than about 25%, preferably less than about 10%, more preferably less than about 5%, even more preferably less than about 2% and even more preferably less than about 1% of the (+)-isomer calculated as

$$\% ( - ) \text{-} enantiomer = \frac{( mass ( - ) \text{-} enantionmer )}{( mass ( + ) \text{-} enantionmer ) + ( mass ( - ) \text{-} enantionmer )} \times 100.$$

It is intended that within the scope of the present invention, any one or more element(s), in particular when mentioned in relation to a compound of Formula (I), shall comprise all isotopes and isotopic mixtures of said element(s), either naturally occurring or synthetically produced, either with natural abundance or in an isotopically enriched form. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. The isotopes may be radioactive or non-radioactive. Radiolabelled compounds of formula (I) may comprise one or more radioactive isotope(s) selected from the group of ³H, ¹¹C, ¹⁸F, ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ²H, ³H, ¹¹C and ¹⁸F.

During any of the processes for preparation of the compounds of the various embodiments of the present invention, it may be necessary and/or desirable to protect sensitive or reactive groups on any of the molecules concerned. This may be achieved by means of conventional protecting groups such as those described in Protective Groups in Organic Chemistry, Second Edition, J. F. W. McOmie, Plenum Press, 1973; T. W. Greene & P. G. M. Wuts, Protective Groups in Organic Synthesis, John Wiley & Sons, 1991; and T. W. Greene & P. G. M. Wuts, Protective Groups in Organic Synthesis, Third Edition, John Wiley & Sons, 1999. The protecting groups may be removed at a convenient subsequent stage using methods known from the art.

The term “room temperature” (RT) refers to a temperature of from about 15° C. to about 30° C., in particular from about 20° C. to about 30° C. Preferably, room temperature is a temperature of about 25° C.

An average molecular weight may, for example, refer to a number average or weight average molecular weight. Average molecular weight may, for example, be measured using gel permeation chromatography.

The term “subject” refers to an animal, preferably a mammal, most preferably a human, who has been the object of treatment, observation or experiment.

The term “therapeutically effective amount” refers to an amount of an active compound or pharmaceutical agent which elicits the biological or medicinal response in a tissue system, animal or human that is being sought by a researcher, veterinarian, medical doctor or other clinician, including reduction or inhibition of an enzyme or a protein activity, or ameliorating symptoms, alleviating conditions, slowing or delaying disease progression, or preventing a disease.

In embodiments in which the active pharmaceutical ingredient is a MALT1 inhibitor, the term “therapeutically effective amount” may refer to the amount of a formulation of the present invention that, when administered to a subject, is effective to (1) at least partially alleviate, inhibit, prevent, and/or ameliorate a condition, or a disorder or a disease (i) mediated by MALT1; or (ii) associated with MALT1 activity; or (iii) characterized by activity (normal or abnormal) of MALT1; or (2) reduce or inhibit the activity of MALT1; or (3) reduce or inhibit the expression of MALT1; or (4) modify the protein levels of MALT1.

The term “MALT1-mediated” refers to any disease, syndrome, condition, or disorder that might occur in the absence of MALT1 but can occur in the presence of MALT1. Suitable examples of a disease, syndrome, condition, or disorder mediated by MALT1 include, but are not limited to, lymphomas, leukemias, carcinomas, and sarcomas, e.g. non-Hodgkin's lymphoma (NHL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocytic leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, and GIST (gastrointestinal stromal tumor).

As used herein, the term “MALT1 inhibitor” refers to an agent that inhibits or reduces at least one condition, symptom, syndrome, disorder, and/or disease of MALT1.

As used herein, unless otherwise noted, the term “affect” or “affected” (when referring to a disease, syndrome, condition or disorder that is affected by the inhibition of MALT1) includes a reduction in the frequency and/or severity of one or more symptoms or manifestations of said disease, syndrome, condition or disorder; and/or includes the prevention of the development of one or more symptoms or manifestations of said disease, syndrome, condition or disorder or the development of the disease, condition, syndrome or disorder.

As used herein, the term “treat”, “treating”, or “treatment” of any disease, condition, syndrome or disorder refers, in one embodiment, to ameliorating the disease, condition, syndrome or disorder (i.e. slowing or arresting or reducing the development of the disease or at least one of the clinical symptoms thereof). In another embodiment, “treat”, “treating”, or “treatment” refers to alleviating or ameliorating at least one physical parameter including those which may not be discernible by the patient. In a further embodiment, “treat”, “treating”, or “treatment” refers to modulating the disease, condition, syndrome or disorder either physically (e.g. stabilization of a discernible symptom), physiologically, (e.g. stabilization of a physical parameter), or both. In yet another embodiment, “treat”, “treating”, or “treatment” refers to preventing or delaying the onset or development or progression of the disease, condition, syndrome or disorder.

### Pharmaceutical Formulations

The invention provides a pharmaceutical formulation, comprising:
a) a polyethylene glycol having a freezing point of at least about 30° C.;
b) an active pharmaceutical ingredient (API) that is soluble in molten polyethylene glycol wherein the polyethylene glycol is as defined in a); and
c) a crystallisation rate inhibitor.
In particular, the invention provides a pharmaceutical formulation, comprising:
a) a polyethylene glycol having a freezing point of at least about 30° C.;
b) an active pharmaceutical ingredient (API) which is

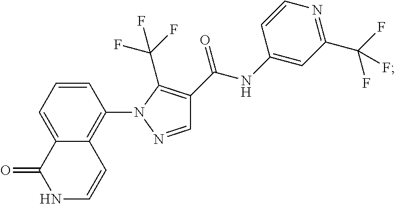 [SMILES: O=C(Nc1ccnc(C(F)(F)F)c1)c1cnn(-c2cccc3c(=O)[nH]ccc23)c1C(F)(F)F]

and
c) a crystallisation rate inhibitor.
In particular, the invention provides a pharmaceutical formulation, comprising:
a) a polyethylene glycol having a freezing point of at least about 30° C.;
b) an active pharmaceutical ingredient (API) which is

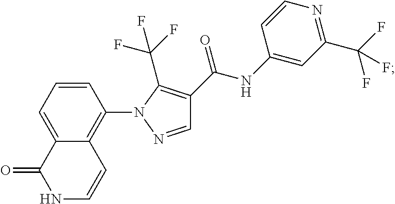 [SMILES: O=C(Nc1ccnc(C(F)(F)F)c1)c1cnn(-c2cccc3c(=O)[nH]ccc23)c1C(F)(F)F]

and
c) a crystallisation rate inhibitor which is PVPVA64.
In an embodiment, the API is soluble in the polyethylene glycol molten at 5° C. above the freezing point of said polyethylene glycol.

The pharmaceutical formulation of the invention may comprise at most about 50 w/w %, at most about 45 w/w %, at most about 40 w/w %, at most about 35 w/w %, or at most about 30 w/w % of the active pharmaceutical ingredient relative to the total weight of the formulation. The pharmaceutical formulation may comprise at least about 0.1 w/w %, at least about 1 w/w %, at least about 5 w/w %, at least about 10 w/w %, or at least about 15 w/w % of the active pharmaceutical ingredient relative to the total weight of the formulation. The pharmaceutical formulation may comprise from about 0.1 w/w % to about 40 w/w %, from about 1 w/w % to about 30 w/w %, or from about 5 w/w % to about 25 w/w % of the active pharmaceutical ingredient relative to the total weight of the formulation. The formulation may comprise from about 20 w/w % to about 25 w/w % of the active pharmaceutical ingredient relative to the total weight of the formulation.

The pharmaceutical formulation of the invention may contain about 0.1 mg to about 3000 mg of the API, or any particular amount or range therein, in particular from about 1 mg to about 1000 mg of the API, or any particular amount or range therein, or, more particularly, from about 10 mg to about 500 mg of the API, or any particular amount or range therein, of API in a regimen of about 1 to about (4×) per day for an average (70 kg) human; although, it is apparent to one skilled in the art that the therapeutically effective amount for said API will vary as will the diseases, syndromes, conditions, and disorders being treated.

The pharmaceutical formulation of the invention comprises polyethylene glycol having a freezing point of at least about 30° C. The polyethylene glycol may have a freezing point of from about 30° C. to about 70° C., from about 35° C. to about 70° C., from about 35° C. to about 65° C., or from about 40° C. to about 60° C. The polyethylene glycol may have a freezing point of from about 35° C. to about 65° C. In a particular embodiment, the pharmaceutical formulation of the invention comprises polyethylene glycol having an upper limit of the freezing point of at least about 30° C. The polyethylene glycol may have an upper limit of the freezing point of from about 30° C. to about 70° C., from about 35° C. to about 70° C., from about 35° C. to about 65° C., or from about 40° C. to about 60° C. The polyethylene glycol may have an upper limit of the freezing point of from about 35° C. to about 65° C. The freezing point may be determined using the procedure provided in 2.2.18 of the European Pharmacopoeia 6^{th} Edition, which is incorporated herein by reference. As an example PEG1500 has a freezing point of 42-48° C., which means that the upper limit of the freezing point is 48° C.

The above freezing points of polyethylene glycol can alternatively be referred to as “melting point”. The above freezing point values and ranges therefore also provide equivalent melting point values and ranges. The polyethylene glycols may also be characterised by melting point.

The polyethylene glycol (PEG) may have an average molecular weight of at least about 900 g/mol or at least about 1000 g/mol. The polyethylene glycol may have an average molecular weight of from about 1000 to about 20000 g/mol, from about 1000 to about 10000 g/mol, or from about 1000 to 5000 g/mol. The polyethylene glycol may have an average molecular weight of at least about 1400 g/mol. The polyethylene glycol may be a PEG grade selected from PEG1000, PEG1450, PEG1500, PEG1540, PEG2000, PEG3000, PEG3350, PEG3400, PEG4000, PEG4600, PEG5500, PEG6000, PEG8000, PEG9000, PEG10000, PEG12000 and PEG20000. The polyethylene glycol may be selected from PEG1500, PEG2000 and PEG3000. The polyethylene glycol may be selected from PEG1500, PEG2000, PEG3350 and PEG 4000. The polyethylene glycol of the invention may comprise a mixture of two or more PEG grades.

As the average molecular weight of polyethylene glycol increases, hygroscopicity decreases, while freezing point increases. The solubility of the API in the polyethylene glycol may also decrease as average molecular weight of polyethylene glycol increases. Therefore, the skilled person can select a polyethylene glycol with an average molecular weight that provides an optimal balance of these properties for any particular API.

Various PEG grades are commercially available. Characterisation of various PEG grades is, for example, provided in the European Pharmacopoeia 6^{th} 1 Edition.

The PEG grades disclosed herein may refer to polyethylene glycols with average molecular weights within a range corresponding to the specified grade as set out in the European Pharmacopoeia 6^{th} Edition. The range of average molecular weights may be at most about +/−10% of the specified grade. For example, PEG1000 may be a polyethylene glycol with an average molecular weight of 950-1050 g/mol. PEG1500 may be a polyethylene glycol with an average molecular weight of 1400-1600 g/mol. PEG2000 may be a polyethylene glycol with an average molecular weight of 1800-2200 g/mol. PEG3000 may be a polyethylene glycol with an average molecular weight of 2700-3300 g/mol. PEG4000 may be a polyethylene glycol with an average molecular weight of 3700-4400 g/mol.

The average molecular weight may be determined using the procedure provided in the US Pharmacopoeia Official Monographs, page information USP42-NF37-5882 (“Polyethylene Glycol, Assay, Average Molecular Weight”) which is incorporated herein by reference.

Names and abbreviations for polyethylene glycol include but are not limited to PEG and macrogol. Macrogol is the international non-proprietary name for polyethylene glycol used in medicine.

The pharmaceutical formulation of the invention may comprise at least about 20 w/w %, at least about 30 w/w %, at least about 40 w/w %, at least about 50 w/w %, at least about 60 w/w %, or at least about 65 w/w % polyethylene glycol relative to the total weight of the formulation. The pharmaceutical formulation may comprise from about 70 w/w % to about 95 w/w %, from about 70 w/w % to about 90 w/w %, from about 70 w/w % to about 85 w/w %, or from about 70 w/w % to about 80 w/w % of polyethylene glycol relative to the total weight of the formulation.

The pharmaceutical formulation of the invention may be a solid dispersion. In particular, the pharmaceutical formulation may be a solid solution. Solid solutions are discussed in Leuner & Dressman, Eur. J Pharm. Biopharm., 50, 2000, 47-60, which is incorporated herein by reference.

The pharmaceutical formulation of the invention also comprises a crystallisation rate inhibitor. A crystallisation rate inhibitor refers to an excipient, for example a polymeric excipient, that is added to the formulation with the aim of inhibiting crystallisation of an API when the formulation is administered to a subject. A crystallisation rate inhibitor may be used to improve the bioavailability of an API where the crystalline form is typically significantly lower in comparison to the amorphous/dissolved state. The crystallisation rate inhibitor may be referred to as a crystallisation inhibitor or a stabilizer.

In an embodiment, the crystallisation rate inhibitor is selected from polyvinylpyrrolidone (PVP), a polyvinylpyrrolidone-vinyl acetate copolymer (PVPVA), a poly(meth)acrylate polymer (e.g. methacrylic acid-methyl methacrylate copolymer), a cyclodextrin or a cyclodextrin derivative (e.g. (2-hydroxypropyl)-β-cyclodextrin (HPBCD)), hydroxypropylcellulose, methylcellulose, hydroxypropyl methylcellulose (HPMC), hydroxypropyl methylcellulose acetate succinate (HPMCAS), a polyethylene glycol-polyvinyl acetate-polyvinyl caprolactame graft copolymer, a poloxamer (e.g. poloxamer 188, 338, or 407), and combinations thereof.

In an embodiment, the crystallisation rate inhibitor is selected from polyvinylpyrrolidone (PVP) and a polyvinylpyrrolidone-vinyl acetate copolymer (PVPVA), and a combination thereof. In a further embodiment, the crystallisation rate inhibitor is selected from polyvinylpyrrolidone-vinyl acetate copolymer (PVPVA). The PVPVA may be a copolymer of 1-vinyl-2-pyrrolidone and vinyl acetate in a ratio of 6:4 by mass (PVPVA64).

Names and abbreviations for polyvinylpyrrolidone-vinyl acetate copolymer include, but are not limited to, PVPVA, PVP-VAc-copolymer, and poly(1-vinylpyrrolidone-co-vinyl-acetate).

Names and abbreviations for a copolymer of 1-vinyl-2-pyrrolidone and vinyl acetate in a ratio of 6:4 by mass (PVPVA64) include, but are not limited to, copolyvidone, copovidum, and copovidone. Examples of commercially available PVPVA64 are Kollidon® VA64, Kollidon® VA64 Fine, Luviskol VA64®, and Plasdone S-630®.

Names and abbreviations for polyvinylpyrrolidone include, but are not limited to, PVP, povidone and crospovidone. Crospovidone is a crosslinked homopolymer of vinyl pyrrolidone.

Examples of commercially available poly(meth)acrylate polymers are Eudragit® polymers. Eudragit® polymers include amino alkyl methacrylate copolymers, methacrylic acid copolymers, methacrylic ester copolymers, and ammonioalkyl methacrylate copolymers. For example, Eudragit® L 100-55 is a copolymer of ethyl acrylate and methacrylic acid.

An example of a commercially available HPBCD is Cavasol®.

An example of a commercially available polyethylene glycol-polyvinyl acetate-polyvinylcaprolactame-based graft copolymer is Soluplus®.

Names and abbreviations for hydroxypropylcellulose include, but are not limited to, hypromellose.

An example of a commercially available HPMC is Methocel®. An example of a commercially available HPMCAS is Affinisol™.

Poloxamers are triblock copolymers based on poly(ethylene oxide) and poly(propylene oxide). Examples of commercially available poloxamers are Pluronic® polymers.

The crystallisation rate inhibitor may be soluble in polyethylene glycol or may form a suspension in polyethylene glycol.

The capsule of the solid dosage form may have the role of the crystallisation rate inhibitor. For example, the capsule might be a HPMC capsule.

The pharmaceutical formulation of the invention may comprise at most about 20 w/w % of the crystallisation rate inhibitor relative to the total weight of the formulation. The pharmaceutical formulation may comprise at least about 0.1 w/w % of the crystallisation rate inhibitor relative to the total weight of the formulation. The pharmaceutical formulation may comprise from about 1 w/w % to about 15 w/w % or from about 1 w/w % to about 10 w/w % of the crystallisation rate inhibitor relative to the total weight of the formulation. The pharmaceutical formulation may comprise about 1 w/w %, about 5 w/w % or about 10 w/w % of the crystallisation rate inhibitor.

Crystallisation inhibition can be important for solid dosage forms, in particular those containing formulations of APIs, the absorption of which is solubility and/or dissolution rate limited, such as APIs belonging to BCS class II or IV. Without being bound to any theory, when a solid dosage form containing polyethylene glycol is administered, the polyethylene glycol component will dissolve in the aqueous environment in the gastrointestinal tract, resulting in an API solvent shift from polyethylene glycol to water. If the API is poorly soluble in water, this leads to a high supersaturation of the API in the aqueous environment, resulting in precipitation. The presence of a crystallisation rate inhibitor can lead to the API precipitating out of solution as an amorphous form rather than a crystalline form. Amorphous forms are generally resolubilised much more quickly than crystalline forms, thus resulting in faster absorption of the API into the blood. Crystallisation rate inhibitors can therefore improve the oral bioavailability of APIs.

The pharmaceutical formulation of the invention optionally comprises an antioxidant. The antioxidant may be selected from tocopherol (vitamin E), thiodipropionic acid, lipoic acid, hydroquinone, phytic acid, monothioglycerol, sodium thioglycolate, thioglycol, vitamin E acetate, beta carotene, butylated hydroxyanisole (BHA), butylated hydroxytoluene (BHT), cysteine, cysteine hydrochloride, propyl gallate (PG), sodium metabisulfite, ascorbyl palmitate, ascorbyl stearate, potassium metabisulfite, disodium EDTA (ethylenediamine tetraacetic acid; also known as disodium edentate), EDTA, erythorbic acid, ethoxyquin, glutathione, gum guaiac, lecithin, TBHQ (tert butyl hydroxyquinone), tartaric acid, citric acid, citric acid monohydrate, methane sulfonic acid, methionine, sodium thiosulfate, sodium sulphite, and a combination thereof.

The antioxidant may be selected from tocopherol (vitamin E), lipoic acid, hydroquinone, monothioglycerol, thioglycol, beta carotene, butylated hydroxyanisole (BHA), butylated hydroxytoluene (BHT), propyl gallate (PG), ascorbyl palmitate, ascorbyl stearate, ethoxyquin, TBHQ (tert butyl hydroxyquinone), and a combination thereof. The antioxidant may be tocopherol (vitamin E) or propyl gallate. The antioxidant may be tocopherol (vitamin E). The antioxidant may be propyl gallate. In a particular embodiment the tocopherol (vitamin E) is all-rac-alpha tocopherol.

The antioxidant may be all-rac-alpha tocopherol.

The pharmaceutical formulation of the invention may comprise from about 0.001 w/w % to about 2 w/w % of antioxidant relative to the total weight of the formulation. The pharmaceutical formulation may comprise from about 0.001 w/w % to about 1 w/w % of antioxidant relative to the total weight of the formulation. The pharmaceutical formulation may comprise from about 0.01 w/w % to about 2 w/w % of antioxidant relative to the total weight of the formulation. The pharmaceutical formulation may comprise from about 0.01 w/w % to about 1 w/w % of antioxidant relative to the total weight of the formulation. The pharmaceutical formulation may comprise from about 0.01 w/w % to about 0.5 w/w % of antioxidant relative to the total weight of the formulation. The pharmaceutical formulation may comprise about 0.01 w/w % or about 0.1 w/w % of antioxidant.

The pharmaceutical formulation of the invention may further comprise one or more pharmaceutically acceptable excipients, as described in more detail herein. Pharmaceutically acceptable excipients include, but are not limited to, disintegrants, binders, diluents, lubricants, stabilizers, osmotic agents, colorants, plasticizers, coatings and the like.

More particularly, suitable pharmaceutical excipients comprise one or more of the following: (i) diluents such as lactose, mannitol, microcrystalline cellulose, dicalcium phosphate, maltodextrin, starch and the like; (ii) binders such as polyvinylpyrrolidone (such as povidone), methylcellulose, hydroxypropyl cellulose, hydroxypropyl methylcellulose (such as METHOCEL® E-5), and the like; (iii) disintegrants such as sodium starch glycolate, croscarmellose sodium, crospovidone, L-HPC (low substituted hydroxypropylcellulose), pregelatinized starch, maize starch and the like; (iv) wetting agents such as surfactants, such as sodium lauryl stearate, docusate sodium, polysorbate 20, polysorbate 80 and the like; (v) lubricants such as magnesium stearate, sodium stearyl fumarate, stearic acid, talc, and the like; (vi) flow promoters or glidants such as colloidal silicon dioxide, talc and the like; and other excipients known to be useful in the preparation of pharmaceutical formulations; (vii) stabilizers such as myristic acid, palmitic acid, stearic acid, cetyl alcohol, cetostearyl alcohol, stearylalcohol, glyceryl distearate, glycerol monostearate, glyceryl dibehenate, hard fat or any combination thereof. Additional suitable pharmaceutical excipients and their properties may be found in texts such as Handbook of Pharmaceutical Excipients, Edited by R. C. Rowe, P. J. Sheskey & P. J. Weller, Sixth Edition (Published by Pharmaceutical Press, a Division of Royal Pharmaceutical Society of Great Britain).

Fillers or diluents for use in the pharmaceutical formulations of the present invention include fillers or diluents typically used in the formulation of pharmaceuticals. Examples of fillers or diluents for use in accordance with the present invention include, but are not limited to, sugars such as lactose, dextrose, glucose, sucrose, cellulose, starches and carbohydrate derivatives, polysaccharides (including dextrates and maltodextrin), polyols (including mannitol, xylitol, and sorbitol), cyclodextrins, calcium carbonates, magnesium carbonates, microcrystalline cellulose, combinations thereof, and the like. In certain preferred embodiments the filler or diluent is lactose, microcrystalline cellulose, or combination thereof. Several types of microcrystalline cellulose are suitable for use in the formulations described herein, for example, microcrystalline cellulose selected from the group consisting of Avicel® types: PH101, PH102, PH103, PH105, PH 112, PHi 13, PH200, PH301, and other types of microcrystalline cellulose, such as silicified microcrystalline cellulose. Several types of lactose are suitable for use in the formulations described herein, for example, lactose selected from the group consisting of anhydrous lactose, lactose monohydrate, lactose fast flo, directly compressible anhydrous lactose, and modified lactose monohydrate.

Binders for use in the pharmaceutical formulations of the present invention include binders commonly used in the formulation of pharmaceuticals. Examples of binders for use in accordance with the present invention include but are not limited to cellulose derivatives (including hydroxypropyl cellulose, hydroxypropyl methylcellulose, methylcellulose, and sodium carboxymethyl cellulose), glycol, sucrose, dextrose, corn syrup, polysaccharides (including acacia, targacanth, guar, alginates and starch), corn starch, pregelatinized starch, modified corn starch, gelatin, polyvinylpyrrolidone, polyethyleneglycol, combinations thereof and the like.

Disintegrants for use in the pharmaceutical formulations of the present invention include disintegrants commonly used in the formulation of pharmaceuticals. Examples of disintegrants for use in accordance with the present invention include but are not limited to starches, and crosslinked starches, celluloses and polymers, combinations thereof and the like. Representative disintegrants include microcrystalline cellulose, croscarmellose sodium, alginic acid, sodium alginate, crosprovidone, cellulose, agar and related gums, sodium starch glycolate, corn starch, potato starch, sodiumstarch glycolate, Veegum HV, methylcellulose, L-HPC (low substituted hydroxypropylcellulose), agar, bentonite, sodium carboxymethylcellulose, calcium carboxymethylcellulose, carboxymethylcellulose, alginic acid, guar gum, maize starch, pregelatinized starch, combinations thereof, and the like.

Lubricants, glidants or anti-tacking agents for use in the pharmaceutical formulations of the present invention include lubricants, glidants and anti-tacking agents commonly used in the formulation of pharmaceuticals. Examples for use in accordance with the present invention include but are not limited to magnesium carbonate, magnesium laurylsulphate, calcium silicate, talc, fumed silicon dioxide, combinations thereof, and the like. Other useful lubricants include but are not limited to magnesium stearate, calcium stearate, stearic acid, sodium stearyl fumarate, sodium lauryl sulphate, magnesium lauryl sulphate, sodium benzoate, colloidal silicon dioxide, magnesium aluminometasillicate (such as Neusilin®), magnesium oxide, magnesium silicate, mineral oil, hydrogenated vegetable oils, waxes, glyceryl behenate, and combinations thereof, and the like.

Surfactants for use in the pharmaceutical formulations of the present invention include surfactants commonly used in the formulation of pharmaceuticals. Examples of surfactants for use in accordance with the present invention include but are not limited to zwitterionic, ionic- and nonionic surfactants or wetting agents commonly used in the formulation of pharmaceuticals, such as ethoxylated castor oil, polyglycolyzed glycerides, acetylated monoglycerides, sorbitan fatty acid esters, poloxamers (e.g. Pluronic®), polyethylene glycol (15)-hydroxystearate (e.g. Solutol®), polyoxyethylene sorbitan fatty acid esters, polyoxyethylene derivatives, monoglycerides or ethoxylated derivatives thereof, diglycerides or polyoxyethylene derivatives thereof, dioctyl sulfosuccinate sodium salt (sodium docusate), sodium laurylsulfate (SLS), cholic acid or derivatives thereof, lecithins, phospholipids, combinations thereof, and the like. Non-ionic surfactants may have an HLB (hydrophile-lipophile balance) value higher than 10.

The pharmaceutical formulations disclosed herein can further comprise one or more flow regulators (or glidants). Flow regulators may be present in powders or granules and are admixed in order to increase their flowability of the formulation during manufacture, particularly in the preparation of tablets produced by pressing powders or granules. Flow regulators which can be employed include, but are not limited to, highly disperse silicon dioxide (Aerosil®) or dried starch.

Tablet dosage forms may further comprise a coating. Suitable coatings are film-forming polymers, such as, for example, those from the group of the cellulose derivatives (such as HPC (hydroxypropylcellulose), HPMC (hydroxypropoxymethylcellulose), MC (methylcellulose), HPMCAS (hydroxypropoxymethylcelluclose acetate succinate)), dextrins, starches, natural gums, such as, for example, gum arabic, xanthans, alginates, polyvinyl alcohol, polymethacrylates and derivatives thereof, such as, for example, Eudragit®, which may be applied to the tablet as solutions or suspensions by means of the various pharmaceutical conventional methods, such as, for example, film coating. The coating is typically applied as a solution/suspension which, in addition to any film-forming polymer present, may further comprise one or more adjuvants, such as hydrophilisers, plasticisers, surfactants, dyes and white pigments, such as, for example, titanium dioxide.

One skilled in the art will readily recognize that the appropriate pharmaceutically acceptable excipients are selected such that they are compatible with other excipients and do not bind with the active pharmaceutical ingredient or cause degradation.

The pharmaceutical formulation of the invention preferably is provided as a solid formulation. Formulations containing polyethylene glycols that are solid at ambient temperature (e.g. polyethylene glycols with a freezing point of at least about 30° C.) are generally expected to have improved stability relative to formulations containing liquid polyethylene glycols (e.g. polyethylene glycols with a freezing point of at most about 25° C.). The reduced mobility of molecules in the solid phase reduces reactivity rates and therefore slows any degradation, compared to molecules in the liquid phase.

The pharmaceutical formulation can be obtained by
a) forming a melt comprising polyethylene glycol having a freezing point of at least about 30° C., an active pharmaceutical ingredient, and a crystallisation rate inhibitor, wherein the forming a melt step comprises heating polyethylene glycol to a temperature above its freezing point; and
b) cooling the melt below the freezing point of the polyethylene glycol.

It will be appreciated that any of the above discussion relating to components of the pharmaceutical formulation may apply to any of the other aspects and embodiments of the invention. For example, any embodiment of a polyethylene glycol, crystallisation rate inhibitor, API, and/or any other component of a pharmaceutical formulation as disclosed herein may be present in combination in a pharmaceutical formulation of the invention.

### Active Pharmaceutical Ingredient

Active pharmaceutical ingredients are those which exert a pharmacological, immunological or metabolic action with a view to restoring, correcting or modifying physiological functions or to make a medical diagnosis. Examples thereof are:
analgesic and anti-inflammatory drugs;
anti-arrhythmic drugs;
antibacterial and antiprotozoal agents;
anti-coagulants;
antidepressants;
anti-diabetic drugs;
anti-epileptic drugs;
antifungal agents;
antihistamines;
anti-hypertensive drugs;
anti-muscarinic agents;
antineoplastic agents and antimetabolites;
anti-migraine drugs;
anti-Parkinsonian drugs;
antipsychotic, hypnotic and sedating agents;
anti-stroke agents;
antitussive;
antivirals;
beta-adrenoceptor blocking;
cardiac inotropic agents;
corticosteroids;
disinfectants;
diuretics;
enzymes;
essential oils;
gastro-intestinal agents;
lipid regulating agents;
local anaesthetics;
opioid analgesics;
parasympathomimetics and anti-dementia drugs;
sex hormones;
stimulating agents;
vasodilators.

The invention provides a pharmaceutical formulation, comprising:

a) a polyethylene glycol having a freezing point of at least about 30° C.;
b) an active pharmaceutical ingredient that is soluble in molten polyethylene glycol wherein the polyethylene glycol is as defined in a); and
c) a crystallisation rate inhibitor.

In an embodiment, the API is soluble in the polyethylene glycol molten at 5° C. above the freezing point of said polyethylene glycol.

The solubility may be measured at a temperature above the freezing point of polyethylene glycol or may be measured using hot stage microscopy.

In particular, the API is sufficiently soluble in the molten polyethylene glycol to enable a therapeutically effective dose of the API to be administered in a formulation of the invention. In particular, the solubility of the API in the formulation is sufficient to ensure long term physical stability in a dissolved state at the desired concentration in the formulation. The concentration of API may be as high as deemed necessary to limit the size of the particular dosage form (e.g. capsule size and number) to be taken by a patient in order to reach the therapeutically effective dose. For example, if a capsule size of at most size 00 (dosage form volume=1 mL) is recommended to allow ease of swallowability and if the estimated targeted therapeutic dose is up to 1 g, 5 capsules of a 200 mg/dosage form formulation per day would be desired for a patient to reach the therapeutically effective targeted dose. Therefore, in this example, the API would have a solubility of at least 200 mg/mL in the formulation. Lower solubility would represent an increase in the number of capsules in order to reach the estimated therapeutically effective dose.

The API may have a solubility of at least about 1, 5, 10, 20, 50, 100, 200, 300, or 350 mg/mL in PEG1500 at a temperature of 50° C. The API may have a solubility of at least about 1, 5, 10, 20, 50, 100, 200, 250, 300, 350 or 400 mg/mL in PEG1500 at a temperature of 50° C. The API may have a solubility ranging from 1-400 mg/mL in PEG1500 at a temperature of 50° C. The API may have a solubility ranging from 1-350 mg/mL in PEG1500 at a temperature of 50° C., in particular ranging from 1-300 mg/mL in PEG1500 at a temperature of 50° C., more in particular ranging from 1-250 mg/mL in PEG1500 at a temperature of 50° C. The API may have a solubility ranging from 20-400 mg/mL in PEG1500 at a temperature of 50° C. The API may have a solubility ranging from 20-350 mg/mL in PEG1500 at a temperature of 50° C., in particular ranging from 20-300 mg/mL in PEG1500 at a temperature of 50° C., more in particular ranging from 20-250 mg/mL in PEG1500 at a temperature of 50° C. The API may have a solubility ranging from 100-400 mg/mL in PEG1500 at a temperature of 50° C. The API may have a solubility ranging from 100-350 mg/mL in PEG1500 at a temperature of 50° C., in particular ranging from 100-300 mg/mL in PEG1500 at a temperature of 50° C., more in particular ranging from 100-250 mg/mL in PEG1500 at a temperature of 50° C. The API may have a solubility of at least about 1, 5, 10, 20, 50, 100, 200, 300, or 350 mg/mL in PEG1500 at a temperature of 53° C. The API may have a solubility of at least about 1, 5, 10, 20, 50, 100, 200, 250, 300, 350 or 400 mg/mL in PEG1500 at a temperature of 53° C. The API may have a solubility ranging from 1-400 mg/mL in PEG1500 at a temperature of 53° C. The API may have a solubility ranging from 1-350 mg/mL in PEG1500 at a temperature of 53° C., in particular ranging from 1-300 mg/mL in PEG1500 at a temperature of 53° C., more in particular ranging from 1-250 mg/mL in PEG1500 at a temperature of 53° C. The API may have a solubility ranging from 20-400 mg/mL in PEG1500 at a temperature of 53° C. The API may have a solubility ranging from 20-350 mg/mL in PEG1500 at a temperature of 53° C., in particular ranging from 20-300 mg/mL in PEG1500 at a temperature of 53° C., more in particular ranging from 20-250 mg/mL in PEG1500 at a temperature of 53° C. The API may have a solubility ranging from 100-400 mg/mL in PEG1500 at a temperature of 53° C. The API may have a solubility ranging from 100-350 mg/mL in PEG1500 at a temperature of 53° C., in particular ranging from 100-300 mg/mL in PEG1500 at a temperature of 53° C., more in particular ranging from 100-250 mg/mL in PEG1500 at a temperature of 53° C. The API may have a solubility of 375-400 mg/mL in a mixture of PEG1500 with 5% PVPVA at temperature of 50° C. The API may have a solubility of 375-400 mg/mL in a mixture of PEG1500 with 5% PVPVA at temperature of 53° C.

Solubility may be measured using a classical shake-flask determination (within a range using visual assessment). This method is typically used for determination at 50° C.

Solubility may be measured using hot stage microscopy or differential scanning microscopy (DSC). This method is typically used for determination of solubility at room temperature.

In an embodiment, the API has poor solubility in water. In an embodiment, the API has a solubility of at most about 50, 20, 10, 1, 0.1, 0.01, or 0.001 mg/mL in water. Solubility may be measured e.g. at 25° C. or 50° C. using the shake-flask method. The API may be defined as sparingly soluble (from 30 to 100 parts water for 1 part API), slightly soluble (from 100 to 1000 parts water for 1 part API), very slightly soluble (from 1000 to 10,000 parts water for 1 part API), or practically insoluble (more than 10,000 parts water for 1 part API) in water, as defined by The Pharmacopeia of the United States of America, in the chapter “General notices and Requirements” (Page information USP42-NF37 2S-9081; Section 5.30 Description and Solubility).

In particular, the API is in amorphous form or dissolved state (i.e. molecular dispersion) in the pharmaceutical formulation.

The active pharmaceutical ingredient (API) may be a MALT1 inhibitor. For example, embodiments of the invention include a pharmaceutical formulation as described herein, wherein the active pharmaceutical ingredient is a compound of Formula (I)

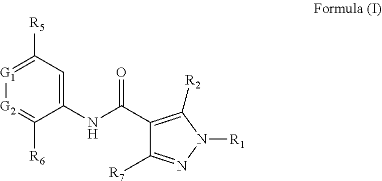

wherein
R₁ is selected from the group consisting of
i) naphthalen-1-yl, optionally substituted with a fluoro or amino substituent; and
ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, ethyl, propyl, isopropyl, trifluoromethyl, cyclopropyl, methoxymethyl, difluoromethyl, 1,1-difluoroethyl, hydroxymethyl, 1-hydroxyethyl, 1-ethoxyethyl, hydroxy, methoxy, ethoxy, fluoro, chloro, bromo, methylthio, cyano, amino, methylamino, dimethylamino, 4-oxotetrahydrofuran-2-yl, 5-oxopyrrolidin-2-yl, 1,4-dioxanyl, aminocarbonyl, methylcarbonyl, methylaminocarbonyl, oxo, 1-(t-butoxycarbonyl)azetidin-2-yl, N-(methyl)formamidomethyl, tetrahydrofuran-2-yl, 3-hydroxy-pyrrolidin-1-yl, pyrrolidin-2-yl, 3-hydroxyazetidinyl, azetidin-3-yl, or azetidin-2-yl;
R₂ is selected from the group consisting of C₁₋₄alkyl, 1-methoxy-ethyl, difluoromethyl, fluoro, chloro, bromo, cyano, and trifluoromethyl;
G₁ is N or C(R₄);
G₂ is N or C(R₃); such that only one of G₁ and G₂ are N in any instance;
R₃ is independently selected from the group consisting of trifluoromethyl, cyano, C₁₋₄alkyl, fluoro, chloro, bromo, methylcarbonyl, methylthio, methylsulfinyl, and methanesulfonyl; or, when G₁ is N, R₃ is further selected from C₁₋₄alkoxycarbonyl;
R₄ is selected from the group consisting of
i) hydrogen, when G₂ is N;
ii) C₁₋₄alkoxy;
iii) cyano;
iv) cyclopropyloxy;
v) a heteroaryl selected from the group consisting of triazolyl, oxazolyl, isoxazolyl, pyrazolyl, pyrrolyl, thiazolyl, tetrazolyl, oxadiazolyl, imidazolyl, 2-amino-pyrimidin-4-yl, 2H-[1,2,3]triazolo[4,5-c]pyridin-2-yl, 2H-[1,2,3]triazolo[4,5-b]pyridin-2-yl, 3H-[1,2,3]triazolo[4,5-b]pyridin-3-yl, 1H-[1,2,3]triazolo[4,5-c]pyridin-1-yl, wherein the heteroaryl is optionally substituted with one or two substituents independently selected from oxo, C₁₋₄alkyl, carboxy, methoxycarbonyl, aminocarbonyl, hydroxymethyl, aminomethyl, (dimethylamino)methyl, amino, methoxymethyl, trifluoromethyl, amino(C₂₋₄alkyl)amino, or cyano;
vi) 1-methyl-piperidin-4-yloxy;
vii) 4-methyl-piperazin-1-ylcarbonyl;
viii) (4-aminobutyl)aminocarbonyl;
ix) (4-amino)butoxy;
x) 4-(4-aminobutyl)-piperazin-1-ylcarbonyl;
xi) methoxycarbonyl;
xii) 5-chloro-6-(methoxycarbonyl)pyridin-3-ylaminocarbonyl;
xiii) 1,1-dioxo-isothiazolidin-2-yl;
xiv) 3-methyl-2-oxo-2,3-dihydro-1H-imidazol-1-yl;
xv) 2-oxopyrrolidin-1-yl;
xvi) (E)-(4-aminobut-1-en-1-yl-aminocarbonyl;
xvii) difluoromethoxy;
and
xviii) morpholin-4-ylcarbonyl;
R₅ is independently selected from the group consisting of hydrogen, chloro, fluoro, bromo, methoxy, methylsulfonyl, cyano, C₁₋₄alkyl, ethynyl, morpholin-4-yl, trifluoromethyl, hydroxyethyl, methylcarbonyl, methylsulfinyl, 3-hydroxy-pyrrolidin-1-yl, pyrrolidin-2-yl, 3-hydroxyazetidinyl, azetidin-3-yl, azetidin-2-yl, methylthio, and 1,1-difluoroethyl;
or R₄ and R₅ may be taken together to form 8-chloro-4-methyl-3-oxo-3,4-dihydro-2H-benzo[b][1,4]oxazin-6-yl, 8-chloro-3-oxo-3,4-dihydro-2H-benzo[b][1,4]oxazin-6-yl, 2-methyl-1-oxo-1,2,3,4-tetrahydroisoquinolin-7-yl, 4-methyl-3-oxo-3,4-dihydro-2H-benzo[b][1,4]oxazin-6-yl, 3-oxo-3,4-dihydro-2H-benzo[b][1,4]oxazin-6-yl, 1-methyl-1H-pyrazolo[3,4-b]pyridin-5-yl, 1H-pyrazolo[3,4-b]pyridin-5-yl, 2,3-dihydro-[1,4]dioxino[2,3-b]pyridin-5-yl, 1,3-dioxolo[4,5]pyridine-5-yl, 1-oxo-1,3-dihydroisobenzofuran-5-yl, 2,2-dimethylbenzo[d][1,3]dioxol-5-yl, 2,3-dihydrobenzo[b][1,4]dioxin-6-yl, 1-oxoisoindolin-5-yl, or 2-methyl-1-oxoisoindolin-5-yl, 1H-indazol-5-yl;
R₆ is hydrogen, C₁₋₄alkyl, fluoro, 2-methoxy-ethoxy, chloro, cyano, or trifluoromethyl; and
R₇ is hydrogen or fluoro;
or an enantiomer, diastereomer, solvate, or pharmaceutically acceptable salt form thereof.

Embodiments of the present invention include a pharmaceutical formulation as described herein, wherein the active pharmaceutical ingredient is a compound of Formula (I)

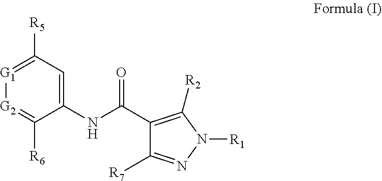

wherein
AA) R₁ is
   i) naphthalen-1-yl, optionally substituted with a fluoro or amino substituent; or
   ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, ethyl, propyl, isopropyl, trifluoromethyl, methoxymethyl, difluoromethyl, 1,1-difluoroethyl, hydroxymethyl, 1-hydroxyethyl, hydroxy, methoxy, fluoro, chloro, bromo, cyano, amino, methylamino, 4-oxotetrahydrofuran-2-yl, 5-oxopyrrolidin-2-yl, 1,4-dioxanyl, aminocarbonyl, methylaminocarbonyl, oxo, N-(methyl)formamidomethyl, tetrahydrofuran-2-yl, 3-hydroxy-pyrrolidin-1-yl, pyrrolidin-2-yl, 3-hydroxyazetidinyl, azetidin-3-yl, or azetidin-2-yl;
BB) R₁ is
   i) naphthalen-1-yl, optionally substituted with a fluoro or amino substituent; or
   ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, difluoromethyl, hydroxymethyl, 1-hydroxyethyl, hydroxy, fluoro, cyano, amino, aminocarbonyl, methylaminocarbonyl, oxo, tetrahydrofuran-2-yl, 3-hydroxy-pyrrolidin-1-yl, pyrrolidin-2-yl, 3-hydroxyazetidinyl, azetidin-3-yl, or azetidin-2-yl;
CC) R₁ is
   i) naphthalen-1-yl, optionally substituted with an amino or fluoro substituent; or
   ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from hydroxymethyl, 1-hydroxyethyl, hydroxy, fluoro, cyano, amino, 3-hydroxyazetidinyl, or oxo;
DD) R₁ is
   i) naphthalen-1-yl, 4-amino-naphthalen-1-yl, 4-fluoronaphthalen-1-yl, or 5-fluoronaphthalen-1-yl;
      or
   ii) a heteroaryl selected from the group consisting of isoquinolin-1-yl, isoquinolin-4-yl, isoquinolin-5-yl, isoquinolin-8-yl, quinolin-7-yl, cinnolin-4-yl, imidazo[1,2-a]pyrazin-8-yl, phthalazin-1-yl, naphthyridin-5-yl, thieno[3,2-c]pyridin-4-yl, furo[3,2-c]pyridin-4-yl, furo[2,3-c]pyridin-7-yl, quinoxalin-5-yl, 1H-indazolylfuro[3,2-b]pyridin-7-yl, pyrazolo[1,5-a]pyrazin-4-yl, quinolin-4-yl, quinolin-5-yl, 1-aminoisoquinolin-4-yl, 1-oxo-1,2-dihydroisoquinolin-5-yl, benzo[d]thiazol-7-yl, 1-hydroxyisoquinolin-5-yl, benzo[d][1,2,3]thiadiazol-7-yl, thieno[2,3-c]pyridin-4-yl, pyrazolo[1,5-a]pyridin-4-yl, thieno[3,2-b]pyridin-7-yl, 2-oxo-1,2-dihydroquinolin-4-yl, 1-amino-8-fluoroisoquinolin-4-yl, 8-fluoroisoquinolin-4-yl, 1-cyanoisoquinolin-5-yl, pyrrolo[2,1-f][1,2,4]triazin-4-yl, 7-(1-hydroxyethyl)thieno[2,3-c]pyridin-4-yl, thieno[2,3-d]pyrimidin-4-yl, thieno[2,3-c]pyridin-7-yl, 1,7-naphthyridin-5-yl, pyrrolo[1,2-a]pyrazin-1-yl, imidazo[1,2-a]pyridin-5-yl, 1-aminocarbonyl-isoquinolin-4-yl, benzo[d]thiazol-4-yl, 8-fluoro-1-hydroxyisoquinolin-4-yl, thieno[3,2-d]pyrimidin-4-yl, 8-fluoroimidazo[1,2-a]pyridin-5-yl, 3-methylimidazo[1,2-a]pyridin-5-yl, 1-oxo-quinolin-4-yl, 8-aminoquinolin-5-yl, benzo[d]oxazol-4-yl, 3-methylthieno[3,2-b]pyridin-7-yl, 1-(hydroxymethyl)isoquinolin-4-yl, (3R-hydroxypyrrolidin-1-yl)isoquinolin-4-yl, (1-hydroxyethyl)isoquinolin-4-yl, 8-fluoroisoquinolin-4-yl, 2-(difluoromethyl)quinolin-4-yl, 8-fluoroquinolin-5-yl, 1-hydroxyisoquinolin-4-yl, 1-(tetrahydrofuran-2-yl)isoquinolin-4-yl, 7-(difluoromethyl)thieno[2,3-c]pyridin-4-yl, 1-(1-hydroxyethyl)isoquinolin-4-yl, 1-cyanoisoquinolin-4-yl, 1-(1(R)-hydroxyethyl)isoquinolin-4-yl, quinazolin-4-yl, 2-methylimidazo[1,2-a]pyridin-3-yl, thiazolo[5,4-d]pyrimidin-7-yl, 6-N-oxido-1H-pyrazol-1-yl)thieno[2,3-c]pyridin-4-yl, imidazo[1,2-a]pyridin-3-yl, furo[2,3-d]pyrimidin-4-yl, 2-fluoroquinolin-5-yl, isoquinolin-5-yl, benzo[d]isothiazol-3-yl, 7-methylpyrazolo[1,5-a]pyridin-4-yl, 1-(hydroxyethyl)quinolin-4-yl, 1-(methoxymethyl)isoquinolin-4-yl, 1-fluoroisoquinolin-4-yl, 1-(difluoromethyl)isoquinolin-4-yl, 8-fluoroquinolin-4-yl, 8-fluoroquinolin-5-yl, 1-(tetrahydrofuran-2(R)-yl)isoquinolin-4-yl, 2-amino-[1,2,4]triazolo[1,5-a]pyridin-5-yl, 1-(4-oxotetrahydrofuran-2-yl)isoquinolin-4-yl, 2-(aminocarbonyl)quinolin-4-yl, 1H-indazol-7-yl, 1-(1,4-dioxan-2-yl)isoquinolin-4-yl, 2-methylimidazo[1,2-a]pyridin-5-yl, 1-chloroisoquinolin-4-yl, 2-cyanoquinolin-4-yl, 8-fluoro-1-(methylamino)isoquinolin-4-yl, benzo[d]isoxazol-3-yl, 2-aminobenzo[d]thiazol-7-yl, 2-fluoroquinolin-5-yl, 1,7-naphthyridin-4-yl, imidazo[1,2-a]pyrazin-5-yl, (N-(methyl)formamido)methyl)isoquinolin-4-yl, [1,2,4]triazolo[1,5-a]pyridin-5-yl, 2-methylbenzo[d]oxazol-7-yl, 1,5-naphthyridin-4-yl, 5-oxopyrrolidin-2-ylisoquinolin-4-yl, 1-methyl-1H-indazol-3-yl, 8-fluoroimidazo[1,2-a]pyridin-5-yl, 1-(tetrahydrofuran-2-yl)isoquinolin-4-yl, 1-(4-oxotetrahydrofuran-2-yl)isoquinolin-4-yl, 1-(1,1-difluoroethyl)isoquinolin-4-yl, 1-(1(*S)-hydroxyethyl)isoquinolin-4-yl, 1-(methylamino)isoquinolin-4-yl, 4-fluoroisoquinolin-1-yl, 1H-pyrazolo[4,3-b]pyridin-7-yl, 5-fluoroquinolin-8-yl, 6-fluoroimidazo[1,2-a]pyridin-5-yl, 2-methylfuro[3,2-b]pyridin-7-yl, 8-(difluoromethyl)quinolin-5-yl, 1-(4-oxotetrahydrofuran-2R-yl)isoquinolin-4-yl, 1-(dimethylamino)isoquinolin-4-yl, 1-methyl-1H-pyrazolo[3,4-c]pyridin-7-yl, 2-methyl-[1,2,4]triazolo[1,5-a]pyridin-5-yl, 2-methoxyquinolin-4-yl, imidazo[1,2-a]pyrimidin-5-yl, 2-(difluoromethyl)thieno[2,3-c]pyridin-4-yl, quinolin-5-yl, 1-(1-ethoxyethyl)isoquinolin-4-yl, 2-(azetidin-2-yl)quinolin-4-yl, 2-methylbenzo[d]thiazol-7-yl, 2-acetylquinolin-4-yl, 1-(methylthio)isoquinolin-4-yl, 2-aminoquinolin-5-yl, 1-methoxyisoquinolin-5-yl, imidazo[1,2-b]pyridazin-6-yl, 1-(pyrrolidin-2-yl)isoquinolin-4-yl, 4-(difluoromethyl)quinolin-5-yl, 1-acetylisoquinolin-5-yl, 2-aminoquinolin-5-yl, 1-(azetidin-2-yl)isoquinolin-4-yl, 1-ethoxyisoquinolin-4-yl, 1-methyl-1H-pyrazolo[3,4-b]pyridin-4-yl, 1-aminoisoquinolin-5-yl, 1-methyl-1H-indazol-4-yl, 2-aminoquinolin-4-yl, 2-oxo-1,2-dihydroquinolin-5-yl, 1-(azetidin-3-yl)isoquinolin-4-yl, 2-methylthieno[3,2-b]pyridin-7-yl, benzo[d][1,2,3]thiadiazol-4-yl, 1-(1(S)-hydroxyethyl)isoquinolin-5-yl, imidazo[1,2-a]pyridin-8-yl, 2-methyl-1-oxo-1,2-dihydroisoquinolin-5-yl, 2-(tetrahydrofuran-2-yl)quinolin-5-yl, 1-(1(R)-hydroxyethyl)isoquinolin-5-yl, 1,6-naphthyridin-4-yl, 1H-pyrazolo[3,4-d]pyrimidin-4-yl, 2-aminocarbonyl-quinolin-5-yl, 2-chloroquinolin-5-yl, 2-chloroquinolin-4-yl, 2-cyanoquinolin-5-yl, 1-aminoisoquinolin-5-yl, 2-methoxyquinolin-5-yl, 2-methylbenzo[d]oxazol-4-yl, 2-(difluoromethyl)quinolin-5-yl, 2-(azetidin-2-yl)quinolin-5-yl, 1-(azetidin-2-yl)isoquinolin-5-yl, 1,5-bis(tetrahydrofuran-2-yl)isoquinolin-4-yl, 1-oxo-1,2-dihydroisoquinolin-4-yl, 2-methyl-1-oxo-1,2-dihydroisoquinolin-4-yl, 1-(3-hydroxyazetidin-1-yl)isoquinolin-4-yl, 8-fluoro-1-(3-hydroxyazetidin-1-yl)isoquinolin-4-yl, (R)-8-fluoro-1-(3-hydroxypyrrolidin-1-yl)isoquinolin-4-yl, (S)-8-fluoro-1-(3-hydroxypyrrolidin-1-yl)isoquinolin-4-yl, 3-hydroxyazetidin-1-yl)thieno[2,3-c]pyridin-4-yl, 8-(3-hydroxyazetidin-1-yl)imidazo[1,2-a]pyridin-5-yl, 7-(3-hydroxyazetidin-1-yl)pyrazolo[1,5-a]pyridin-4-yl, 1-(3-hydroxyazetidin-1-yl)isoquinolin-5-yl, and 1-(1-t-butoxycarbonylazetidin-2-yl)isoquinolin-5-yl;
EE) R₁ is
   i) naphthalen-1-yl or 4-fluoronaphthalen-1-yl, 4-amino-naphthalen-1-yl or 5-fluoronaphthalen-1-yl;
      or
   ii) a heteroaryl selected from the group consisting of thieno[3,2-c]pyridin-4-yl, isoquinolin-4-yl, 8-fluoroquinolin-4-yl, furo[3,2-c]pyridin-4-yl, quinolin-5-yl, furo[2,3-c]pyridin-7-yl, benzofuran-4-yl 1,7-naphthyridin-5-yl, pyrrolo[1,2-a]pyrazin-1-yl, imidazo[1,2-a]pyridin-5-yl, 1-aminocarbonyl-isoquinolin-4-yl, pyrrolo[1,2-a]pyrazin-1-yl, benzo[d]thiazol-4-yl, 8-fluoro-1-hydroxyisoquinolin-4-yl, thieno[3,2-d]pyrimidin-4-yl, 8-fluoroimidazo[1,2-a]pyridin-5-yl, 3-methylimidazo[1,2-a]pyridin-5-yl, 1-aminoisoquinolin-4-yl, 1-oxo-quinolin-4-yl, 8-aminoquinolin-5-yl, benzo[d]oxazol-4-yl, 3-methylthieno[3,2-b]pyridin-7-yl, 1-(hydroxymethyl)isoquinolin-4-yl, (3R-hydroxypyrrolidin-1-yl)isoquinolin-4-yl, (1-hydroxyethyl)isoquinolin-4-yl, 8-fluoroisoquinolin-4-yl, 2-(difluoromethyl)quinolin-4-yl, 8-fluoroquinolin-5-yl, 1-hydroxyisoquinolin-4-yl, benzo[d]thiazol-4-yl, 1-aminoisoquinolin-4-yl, 1-(tetrahydrofuran-2-yl)isoquinolin-4-yl, 7-(difluoromethyl)thieno[2,3-c]pyridin-4-yl, 1-(1-hydroxyethyl)isoquinolin-4-yl, 1-cyanoisoquinolin-4-yl, 1-(1(R)-hydroxyethyl)isoquinolin-4-yl, quinazolin-4-yl, 2-methylimidazo[1,2-a]pyridin-3-yl, thiazolo[5,4-d]pyrimidin-7-yl, imidazo[1,2-a]pyridin-5-yl, benzo[d][1,2,3]thiadiazol-7-yl, 6-N-oxido-1H-pyrazol-1-yl)thieno[2,3-c]pyridin-4-yl, imidazo[1,2-a]pyridin-3-yl, furo[2,3-d]pyrimidin-4-yl, 2-fluoroquinolin-5-yl, isoquinolin-5-yl, benzo[d]isothiazol-3-yl, 7-methylpyrazolo[1,5-a]pyridin-4-yl, 1-oxo-1,2-dihydroisoquinolin-4-yl, 2-methyl-1-oxo-1,2-dihydroisoquinolin-4-yl, 1-(3-hydroxyazetidin-1-yl)isoquinolin-4-yl, 8-fluoro-1-(3-hydroxyazetidin-1-yl)isoquinolin-4-yl, (R)-8-fluoro-1-(3-hydroxypyrrolidin-1-yl)isoquinolin-4-yl, (S)-8-fluoro-1-(3-hydroxypyrrolidin-1-yl)isoquinolin-4-yl, 3-hydroxyazetidin-1-yl)thieno[2,3-c]pyridin-4-yl, 8-(3-hydroxyazetidin-1-yl)imidazo[1,2-a]pyridin-5-yl, 7-(3-hydroxyazetidin-1-yl)pyrazolo[1,5-a]pyridin-4-yl, 1-(3-hydroxyazetidin-1-yl)isoquinolin-5-yl, and 1-(hydroxyethyl)quinolin-4-yl;
FF) R₂ is independently selected from the group consisting of methyl, isopropyl, cyano, bromo, chloro, and trifluoromethyl;
GG) R₂ is independently selected from the group consisting of methyl, isopropyl, cyano, and trifluoromethyl;
HH) R₂ is trifluoromethyl;
II) R₃ is independently selected from the group consisting of trifluoromethyl, cyano, methylcarbonyl, methylthio, methylsulfinyl, methanesulfonyl, and chloro; or, when G₁ is N, R₃ is further selected from C₁₋₄alkoxycarbonyl;
JJ) R₃ is independently selected from the group consisting of trifluoromethyl, cyano, and chloro;
KK) G₂ is N or C(R₃), wherein R₃ is chloro;
LL) G₂ is N;
MM) R₄ is selected from the group consisting of
   i) hydrogen, when G₂ is N;
   ii) C₁₋₄alkoxy;
   iii) cyano;
   iv) cyclopropyloxy;
   v) carboxy;
   vi) a heteroaryl selected from the group consisting of triazolyl, oxazolyl, pyrazolyl, thiazolyl, oxadiazolyl, imidazolyl, and pyrimidin-4-yl, wherein the heteroaryl is optionally substituted with one or two substituents independently selected from the group consisting of C₁₋₄alkyl, carboxy, methoxycarbonyl, hydroxymethyl, aminocarbonyl, (dimethylamino)methyl, amino, methoxymethyl, trifluoromethyl, amino(C₂₋₄alkyl)amino, and cyano;
   vii) 1-methyl-piperidin-4-yloxy;
   viii) 4-methyl-piperazin-1-ylcarbonyl;
   ix) (4-aminobutyl)aminocarbonyl;
   x) (4-amino)butoxy;
   xi) methoxycarbonyl;
   xii) 5-chloro-6-(methoxycarbonyl)pyridin-3-ylaminocarbonyl;
   xiii) 1,1-dioxo-isothiazolidin-2-yl;
   and
   xiv) morpholin-4-ylcarbonyl;
NN) R₄ is selected from the group consisting of
   i) hydrogen;
   ii) C₁₋₄alkoxy;
   iii) cyano;
   iv) cyclopropyloxy;
   v) a heteroaryl selected from the group consisting of triazolyl, oxazolyl, pyrazolyl, thiazolyl, oxadiazolyl, and imidazolyl, wherein the heteroaryl is optionally substituted with one or two substituents independently selected from the group consisting of methyl, carboxy, methoxycarbonyl, hydroxymethyl, aminocarbonyl, (dimethylamino)methyl, and amino, methoxymethyl;
   vi) (4-amino)butoxy;
   vii) methoxycarbonyl;
   viii) 5-chloro-6-(methoxycarbonyl)pyridin-3-ylaminocarbonyl;
      and
   ix) 1,1-dioxo-isothiazolidin-2-yl;
OO) R₄ is selected from the group consisting of
   i) methoxy;
   ii) a heteroaryl independently selected from the group consisting of 2H-1,2,3-triazol-2-yl, 4-carboxy-2H-1,2,3-triazol-2-yl, 4-(hydroxymethyl)-2H-1,2,3-triazol-2-yl, 4-methyl-2H-1,2,3-triazol-2-yl, oxazol-2-yl, 4-amino-2H-1,2,3-triazol-2-yl, 4-(hydroxymethyl)-1H-pyrazol-1-yl, 4-(hydroxymethyl)-2H-1,2,3-triazol-2-yl, 4-((dimethylamino)methyl)-2H-1,2,3-triazol-2-yl, 4-methoxycarbonyl-2H-1,2,3-triazol-2-yl, 4-aminocarbonyl-2H-1,2,3-triazol-2-yl, 1-methyl-1H-pyrazol-3-yl, 1,3,4-oxadiazol-2-yl, 2-methyl-2H-tetrazol-5-yl, 5-amino-1-methyl-1H-pyrazol-3-yl, 4-(hydroxymethyl)-1H-pyrazol-1-yl, 4-cyano-2H-1,2,3-triazol-2-yl, 5-amino-1H-1,2,3-triazol-1-yl, 2H-1,2,3-triazol-4-yl, 2H-tetrazol-5-yl, 4-(aminomethyl)-1H-pyrazol-1-yl, 4-(methoxymethyl)-2H-1,2,3-triazol-2-yl, 2-methyl-2H-tetrazol-5-yl, and 4-methyl-1H-1,2,3-triazol-1-yl;
      and
   iii) methoxycarbonyl;
PP) R₄ is independently selected from the group consisting of 2H-1,2,3-triazol-2-yl, 4-carboxy-2H-1,2,3-triazol-2-yl, 4-(hydroxymethyl)-2H-1,2,3-triazol-2-yl, 4-methyl-2H-1,2,3-triazol-2-yl, oxazol-2-yl, 1H-imidazol-2-yl, 4-amino-2H-1,2,3-triazol-2-yl, 4-(hydroxymethyl)-1H-pyrazol-1-yl, 4-(hydroxymethyl)-2H-1,2,3-triazol-2-yl, 4-((dimethylamino)methyl)-2H-1,2,3-triazol-2-yl, 4-methoxycarbonyl-2H-1,2,3-triazol-2-yl, 4-aminocarbonyl-2H-1,2,3-triazol-2-yl, 1-methyl-1H-pyrazol-3-yl, and 1,3,4-oxadiazol-2-yl;
QQ) R₅ is hydrogen, chloro, fluoro, bromo, cyano, methyl, ethyl, or trifluoromethyl; or, R₄ and R₅ may be taken together to form 8-chloro-4-methyl-3-oxo-3,4-dihydro-2H-benzo[b][1,4]oxazin-6-yl or 8-chloro-3-oxo-3,4-dihydro-2H-benzo[b][1,4]oxazin-6-yl;
   RR) R₅ is hydrogen, chloro, bromo, cyano, or trifluoromethyl; or, R₄ and R₅ may be taken together to form 8-chloro-4-methyl-3-oxo-3,4-dihydro-2H-benzo[b][1,4]oxazin-6-yl or 8-chloro-3-oxo-3,4-dihydro-2H-benzo[b][1,4]oxazin-6-yl;
SS) R₅ is hydrogen, chloro, bromo, or cyano;
TT) R₅ is hydrogen, chloro, or cyano;
UU) R₆ is hydrogen or methyl;
VV) R₇ is hydrogen;

and any combination of embodiments AA) though VV) above, provided it is understood that combinations in which different embodiments of the same substituent would be combined are excluded; such that only one of G₁ and G₂ are N in any instance; or an enantiomer, diastereomer, solvate, or pharmaceutically acceptable salt form thereof.

Embodiments of the present invention include a pharmaceutical formulation as described herein wherein the active pharmaceutical ingredient is a compound of Formula (I)

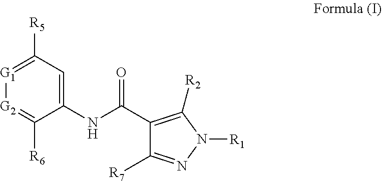

wherein

R₁ is selected from the group consisting of
i) naphthalen-1-yl, 4-amino-naphthalen-1-yl, or 4-fluoronaphthalen-1-yl, 5-fluoronaphthalen-1-yl;
   and
ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, ethyl, propyl, isopropyl, trifluoromethyl, methoxymethyl, difluoromethyl, 1,1-difluoroethyl, hydroxymethyl, 1-hydroxyethyl, hydroxy, methoxy, fluoro, chloro, bromo, cyano, amino, methylamino, 4-oxotetrahydrofuran-2-yl, 5-oxopyrrolidin-2-yl, 1,4-dioxanyl, aminocarbonyl, methylaminocarbonyl, oxo, N-(methyl)formamidomethyl, tetrahydrofuran-2-yl, 3-hydroxy-pyrrolidin-1-yl, pyrrolidin-2-yl, 3-hydroxyazetidinyl, azetidin-3-yl, or azetidin-2-yl;

R₂ is independently selected from the group consisting of methyl, isopropyl, cyano, bromo, chloro, and trifluoromethyl;

G₁ is N or C(R₄);

G₂ is N or C(R₃); such that only one of G₁ and G₂ is N in any instance;

R₃ is independently selected from the group consisting of trifluoromethyl, cyano, methylcarbonyl, methylthio, methylsulfinyl, methanesulfonyl, and chloro; or, when G₁ is N, R₃ is further selected from C₁₋₄alkoxycarbonyl;

R₄ is independently selected from the group consisting of
i) hydrogen, when G₂ is N;
ii) C₁₋₄alkoxy;
iii) cyano;
iv) cyclopropyloxy;
v) carboxy;
vi) a heteroaryl selected from the group consisting of triazolyl, oxazolyl, pyrazolyl, thiazolyl, oxadiazolyl, imidazolyl, and pyrimidin-4-yl, wherein the heteroaryl is optionally substituted with one or two substituents independently selected from the group consisting of C₁₋₄alkyl, carboxy, methoxycarbonyl, hydroxymethyl, aminocarbonyl, (dimethylamino)methyl, amino, methoxymethyl, trifluoromethyl, amino(C₂₋₄alkyl)amino, and cyano;
vii) 1-methyl-piperidin-4-yloxy;
viii) 4-methyl-piperazin-1-ylcarbonyl;
ix) (4-aminobutyl)aminocarbonyl;
x) (4-amino)butoxy;
xi) methoxycarbonyl;
xii) 5-chloro-6-(methoxycarbonyl)pyridin-3-ylaminocarbonyl;
xiii) 1,1-dioxo-isothiazolidin-2-yl;

and
xiv) morpholin-4-ylcarbonyl;

R₅ is hydrogen, chloro, fluoro, bromo, cyano, methyl, ethyl, or trifluoromethyl; or, R₄ and R₅ may be taken together to form 8-chloro-4-methyl-3-oxo-3,4-dihydro-2H-benzo[b][1,4]oxazin-6-yl or 8-chloro-3-oxo-3,4-dihydro-2H-benzo[b][1,4]oxazin-6-yl;

R₆ is hydrogen or methyl; and

R₇ is hydrogen;

or an enantiomer, diastereomer, solvate, or pharmaceutically acceptable salt form thereof.

Embodiments of the present invention include a pharmaceutical formulation as described herein, wherein the active pharmaceutical ingredient is a compound of Formula (I)

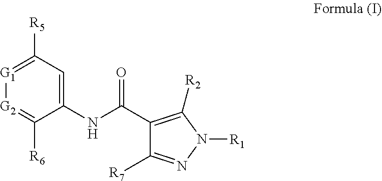

wherein

R₁ is selected from the group consisting of
i) naphthalen-1-yl, optionally substituted with a fluoro or amino substituent; or
ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, difluoromethyl, hydroxymethyl, 1-hydroxyethyl, hydroxy, fluoro, cyano, amino, aminocarbonyl, methylaminocarbonyl, oxo, tetrahydrofuran-2-yl, 3-hydroxy-pyrrolidin-1-yl, pyrrolidin-2-yl, 3-hydroxyazetidinyl, azetidin-3-yl, or azetidin-2-yl;

R₂ is selected from the group consisting of methyl, isopropyl, cyano, and trifluoromethyl;

G₁ is N or C(R₄);

G₂ is N or C(R₃); such that only one of G₁ and G₂ is N in any instance;

R₃ is independently selected from the group consisting of trifluoromethyl, cyano, and chloro;

R₄ is independently selected from the group consisting of
i) hydrogen;
ii) C₁₋₄alkoxy;
iii) cyano;
iv) cyclopropyloxy;
v) a heteroaryl selected from the group consisting of triazolyl, oxazolyl, pyrazolyl, thiazolyl, oxadiazolyl, and imidazolyl, wherein the heteroaryl is optionally substituted with one or two substituents independently selected from the group consisting of methyl, carboxy, methoxycarbonyl, hydroxymethyl, aminocarbonyl, (dimethylamino)methyl, and amino, methoxymethyl;
vi) (4-amino)butoxy;
vii) methoxycarbonyl;
viii) 5-chloro-6-(methoxycarbonyl)pyridin-3-ylaminocarbonyl;
   and
ix) 1,1-dioxo-isothiazolidin-2-yl;

R₅ is hydrogen, chloro, bromo, or cyano;

R₆ is hydrogen or methyl;

R₇ is hydrogen;

or an enantiomer, diastereomer, solvate, or pharmaceutically acceptable salt form thereof.

In some embodiments, a compound of Formula (I) is other than:

a compound wherein R₁ is isoquinolin-8-yl, R₂ is trifluoromethyl, G₁ is C(R₄) wherein R₄ is 2H-1,2,3-triazol-2-yl, G₂ is N, and R₅ is hydrogen;

a compound wherein R₁ is isoquinolin-8-yl, R₂ is trifluoromethyl, G₁ is C(R₄) wherein R₄ is 1H-imidazol-1-yl, G₂ is N, and R₅ is chloro;

a compound wherein R₁ is isoquinolin-8-yl, R₂ is trifluoromethyl, G₁ is C(R₄) wherein R₄ is 1H-1,2,3-triazol-1-yl, G₂ is N, and R₅ is hydrogen; and

a compound wherein R₁ is isoquinolin-8-yl, R₂ is trifluoromethyl, G₁ is C(R₄) wherein R₄ is hydrogen, G₂ is N, and R₅ is fluoro.

Embodiments of the present invention include a pharmaceutical formulation as described herein wherein the active pharmaceutical ingredient is a compound of Formula (I)

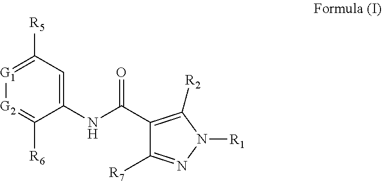

wherein

R₁ is selected from the group consisting of
i) naphthalen-1-yl, optionally substituted with a fluoro or amino substituent; and
ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from hydroxymethyl, 1-hydroxyethyl, hydroxy, fluoro, cyano, amino, oxo, 3-hydroxy-pyrrolidin-1-yl, pyrrolidin-2-yl, 3-hydroxyazetidinyl, azetidin-3-yl, or azetidin-2-yl;

R₂ is selected from the group consisting of methyl, isopropyl, cyano, and trifluoromethyl;
G₁ is N or C(R₄);
G₂ is N or C(R₃); such that only one of G₁ and G₂ is N in any instance;

R₃ is independently selected from the group consisting of trifluoromethyl, cyano, and chloro;

R₄ is selected from the group consisting of
i) methoxy;
ii) a heteroaryl selected from the group consisting of 2H-1,2,3-triazol-2-yl, 4-carboxy-2H-1,2,3-triazol-2-yl, 4-(hydroxymethyl)-2H-1,2,3-triazol-2-yl, 4-methyl-2H-1,2,3-triazol-2-yl, oxazol-2-yl, 4-amino-2H-1,2,3-triazol-2-yl, 4-(hydroxymethyl)-1H-pyrazol-1-yl, 4-(hydroxymethyl)-2H-1,2,3-triazol-2-yl, 4-((dimethylamino)methyl)-2H-1,2,3-triazol-2-yl, 4-methoxycarbonyl-2H-1,2,3-triazol-2-yl, 4-aminocarbonyl-2H-1,2,3-triazol-2-yl, 1-methyl-1H-pyrazol-3-yl, 1,3,4-oxadiazol-2-yl, 2-methyl-2H-tetrazol-5-yl, 5-amino-1-methyl-1H-pyrazol-3-yl, 4-(hydroxymethyl)-1H-pyrazol-1-yl, 4-cyano-2H-1,2,3-triazol-2-yl, 5-amino-1H-1,2,3-triazol-1-yl, 2H-1,2,3-triazol-4-yl, 2H-tetrazol-5-yl, 4-(aminomethyl)-1H-pyrazol-1-yl, 4-(methoxymethyl)-2H-1,2,3-triazol-2-yl, 2-methyl-2H-tetrazol-5-yl, and 4-methyl-1H-1,2,3-triazol-1-yl;

and
iii) methoxycarbonyl;

R₅ is hydrogen, chloro, or cyano;

R₆ is hydrogen or methyl;

R₇ is hydrogen;

or an enantiomer, diastereomer, solvate, or pharmaceutically acceptable salt form thereof.

Embodiments of the present invention include a pharmaceutical formulation as described herein wherein the active pharmaceutical ingredient is a compound of Formula (I)

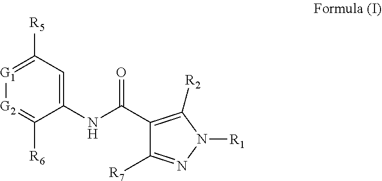

wherein

R₁ is independently selected from the group consisting of
i) naphthalen-1-yl, 4-amino-naphthalen-1-yl, 4-fluoronaphthalen-1-yl, or 5-fluoronaphthalen-1-yl;

and
ii) a heteroaryl selected from the group consisting of isoquinolin-1-yl, isoquinolin-4-yl, isoquinolin-5-yl, isoquinolin-8-yl, quinolin-7-yl, cinnolin-4-yl, imidazo[1,2-a]pyrazin-8-yl, phthalazin-1-yl, naphthyridin-5-yl, thieno[3,2-c]pyridin-4-yl, furo[3,2-c]pyridin-4-yl, furo[2,3-c]pyridin-7-yl, quinoxalin-5-yl, 1H-indazolylfuro[3,2-b]pyridin-7-yl, pyrazolo[1,5-a]pyrazin-4-yl, quinolin-4-yl, quinolin-5-yl, 1-aminoisoquinolin-4-yl, 1-oxo-1,2-dihydroisoquinolin-5-yl, benzo[d]thiazol-7-yl, 1-hydroxyisoquinolin-5-yl, benzo[d][1,2,3]thiadiazol-7-yl, thieno[2,3-c]pyridin-4-yl, pyrazolo[1,5-a]pyridin-4-yl, thieno[3,2-b]pyridin-7-yl, 2-oxo-1,2-dihydroquinolin-4-yl, 1-amino-8-fluoroisoquinolin-4-yl, 8-fluoroisoquinolin-4-yl, 1-cyanoisoquinolin-5-yl, pyrrolo[2,1-f][1,2,4]triazin-4-yl, 7-(1-hydroxyethyl)thieno[2,3-c]pyridin-4-yl, thieno[2,3-d]pyrimidin-4-yl, thieno[2,3-c]pyridin-7-yl, 1,7-naphthyridin-5-yl, pyrrolo[1,2-a]pyrazin-1-yl, imidazo[1,2-a]pyridin-5-yl, 1-aminocarbonyl-isoquinolin-4-yl, benzo[d]thiazol-4-yl, 8-fluoro-1-hydroxyisoquinolin-4-yl, thieno[3,2-d]pyrimidin-4-yl, 8-fluoroimidazo[1,2-a]pyridin-5-yl, 3-methylimidazo[1,2-a]pyridin-5-yl, 1-oxo-quinolin-4-yl, 8-aminoquinolin-5-yl, benzo[d]oxazol-4-yl, 3-methylthieno[3,2-b]pyridin-7-yl, 1-(hydroxymethyl)isoquinolin-4-yl, (3R-hydroxypyrrolidin-1-yl)isoquinolin-4-yl, (1-hydroxyethyl)isoquinolin-4-yl, 8-fluoroisoquinolin-4-yl, 2-(difluoromethyl)quinolin-4-yl, 8-fluoroquinolin-5-yl, 1-hydroxyisoquinolin-4-yl, 1-(tetrahydrofuran-2-yl)isoquinolin-4-yl, 7-(difluoromethyl)thieno[2,3-c]pyridin-4-yl, 1-(1-hydroxyethyl)isoquinolin-4-yl, 1-cyanoisoquinolin-4-yl, 1-(1(R)-hydroxyethyl)isoquinolin-4-yl, quinazolin-4-yl, 2-methylimidazo[1,2-a]pyridin-3-yl, thiazolo[5,4-d]pyrimidin-7-yl, 6-N-oxido-1H-pyrazol-1-yl)thieno[2,3-c]pyridin-4-yl, imidazo[1,2-a]pyridin-3-yl, furo[2,3-d]pyrimidin-4-yl, 2-fluoroquinolin-5-yl, isoquinolin-5-yl, benzo[d]isothiazol-3-yl, 7-methylpyrazolo[1,5-a]pyridin-4-yl, 1-(hydroxyethyl)quinolin-4-yl, 1-(methoxymethyl)isoquinolin-4-yl, 1-fluoroisoquinolin-4-yl, 1-(difluoromethyl)isoquinolin-4-yl, 8-fluoroquinolin-4-yl, 8-fluoroquinolin-5-yl, 1-(tetrahydrofuran-2(R)-yl)isoquinolin-4-yl, 2-amino-[1,2,4]triazolo[1,5-a]pyridin-5-yl, 1-(4-oxotetrahydrofuran-2-yl)isoquinolin-4-yl, 2-(aminocarbonyl)quinolin-4-yl, 1H-indazol-7-yl, 1-(1,4-dioxan-2-yl)isoquinolin-4-yl, 2-methylimidazo[1,2-a]pyridin-5-yl, 1-chloroisoquinolin-4-yl, 2-cyanoquinolin-4-yl, 8-fluoro-1-(methylamino)isoquinolin-4-yl, benzo[d]isoxazol-3-yl, 2-aminobenzo[d]thiazol-7-yl, 2-fluoroquinolin-5-yl, 1,7-naphthyridin-4-yl, imidazo[1,2-a]pyrazin-5-yl, (N-(methyl)formamido)methyl)isoquinolin-4-yl, [1,2,4]triazolo[1,5-a]pyridin-5-yl, 2-methylbenzo[d]oxazol-7-yl, 1,5-naphthyridin-4-yl, 5-oxopyrrolidin-2-ylisoquinolin-4-yl, 1-methyl-1H-indazol-3-yl, 8-fluoroimidazo[1,2-a]pyridin-5-yl, 1-(tetrahydrofuran-2-yl)isoquinolin-4-yl, 1-(4-oxotetrahydrofuran-2-yl)isoquinolin-4-yl, 1-(1,1-difluoroethyl)isoquinolin-4-yl, 1-(1(^{x}S)-hydroxyethyl)isoquinolin-4-yl, 1-(methylamino)isoquinolin-4-yl, 4-fluoroisoquinolin-1-yl, 1H-pyrazolo[4,3-b]pyridin-7-yl, 5-fluoroquinolin-8-yl, 6-fluoroimidazo[1,2-a]pyridin-5-yl, 2-methylfuro[3,2-b]pyridin-7-yl, 8-(difluoromethyl)quinolin-5-yl, 1-(4-oxotetrahydrofuran-2R-yl)isoquinolin-4-yl, 1-(dimethylamino)isoquinolin-4-yl, 1-methyl-1H-pyrazolo[3,4-c]pyridin-7-yl, 2-methyl-[1,2,4]triazolo[1,5-a]pyridin-5-yl, 2-methoxyquinolin-4-yl, imidazo[1,2-a]pyrimidin-5-yl, 2-(difluoromethyl)thieno[2,3-c]pyridin-4-yl, quinolin-5-yl, 1-(1-ethoxyethyl)isoquinolin-4-yl, 2-(azetidin-2-yl)quinolin-4-yl, 2-methylbenzo[d]thiazol-7-yl, 2-acetylquinolin-4-yl, 1-(methylthio)isoquinolin-4-yl, 2-aminoquinolin-5-yl, 1-methoxyisoquinolin-5-yl, imidazo[1,2-b]pyridazin-6-yl, 1-(pyrrolidin-2-yl)isoquinolin-4-yl, 4-(difluoromethyl)quinolin-5-yl, 1-acetylisoquinolin-5-yl, 2-aminoquinolin-5-yl, 1-(azetidin-2-yl)isoquinolin-4-yl, 1-ethoxyisoquinolin-4-yl, 1-methyl-1H-pyrazolo[3,4-b]pyridin-4-yl, 1-aminoisoquinolin-5-yl, 1-methyl-1H-indazol-4-yl, 2-aminoquinolin-4-yl, 2-oxo-1,2-dihydroquinolin-5-yl, 1-(azetidin-3-yl)isoquinolin-4-yl, 2-methylthieno[3,2-b]pyridin-7-yl, benzo[d][1,2,3]thiadiazol-4-yl, 1-(1(S)-hydroxyethyl)isoquinolin-5-yl, imidazo[1,2-a]pyridin-8-yl, 2-methyl-1-oxo-1,2-dihydroisoquinolin-5-yl, 2-(tetrahydrofuran-2-yl)quinolin-5-yl, 1-(1(R)-hydroxyethyl)isoquinolin-5-yl, 1,6-naphthyridin-4-yl, 1H-pyrazolo[3,4-d]pyrimidin-4-yl, 2-aminocarbonyl-quinolin-5-yl, 2-chloroquinolin-5-yl, 2-chloroquinolin-4-yl, 2-cyanoquinolin-5-yl, 1-aminoisoquinolin-5-yl, 2-methoxyquinolin-5-yl, 2-methylbenzo[d]oxazol-4-yl, 2-(difluoromethyl)quinolin-5-yl, 2-(azetidin-2-yl)quinolin-5-yl, 1-(azetidin-2-yl)isoquinolin-5-yl, 1,5-bis(tetrahydrofuran-2-yl)isoquinolin-4-yl, 1-oxo-1,2-dihydroisoquinolin-4-yl, 2-methyl-1-oxo-1,2-dihydroisoquinolin-4-yl, 1-(3-hydroxyazetidin-1-yl)isoquinolin-4-yl, 8-fluoro-1-(3-hydroxyazetidin-1-yl)isoquinolin-4-yl, (R)-8-fluoro-1-(3-hydroxypyrrolidin-1-yl)isoquinolin-4-yl, (S)-8-fluoro-1-(3-hydroxypyrrolidin-1-yl)isoquinolin-4-yl, 3-hydroxyazetidin-1-yl)thieno[2,3-c]pyridin-4-yl, 8-(3-hydroxyazetidin-1-yl)imidazo[1,2-a]pyridin-5-yl, 7-(3-hydroxyazetidin-1-yl)pyrazolo[1,5-a]pyridin-4-yl, 1-(3-hydroxyazetidin-1-yl)isoquinolin-5-yl, and 1-(1-t-butoxycarbonylazetidin-2-yl)isoquinolin-5-yl;

R₂ is trifluoromethyl;

G₁ is N or C(R₄);

G₂ is N or C(R₃); such that only one of G₁ and G₂ is N in any instance;

R₃ is independently selected from the group consisting of trifluoromethyl, cyano, and chloro;

R₄ is independently selected from the group consisting of 2H-1,2,3-triazol-2-yl, 4-carboxy-2H-1,2,3-triazol-2-yl, 4-(hydroxymethyl)-2H-1,2,3-triazol-2-yl, 4-methyl-2H-1,2,3-triazol-2-yl, oxazol-2-yl, 1H-imidazol-2-yl, 4-amino-2H-1,2,3-triazol-2-yl, 4-(hydroxymethyl)-1H-pyrazol-1-yl, 4-(hydroxymethyl)-2H-1,2,3-triazol-2-yl, 4-((dimethylamino)methyl)-2H-1,2,3-triazol-2-yl, 4-methoxycarbonyl-2H-1,2,3-triazol-2-yl, 4-aminocarbonyl-2H-1,2,3-triazol-2-yl, 1-methyl-1H-pyrazol-3-yl, and 1,3,4-oxadiazol-2-yl;

R₅ is hydrogen, chloro, bromo, or cyano;

R₆ is hydrogen or methyl;

R₇ is hydrogen;

or an enantiomer, diastereomer, solvate, or pharmaceutically acceptable salt form thereof.

Embodiments of the present invention include pharmaceutical formulations as described herein wherein the active pharmaceutical ingredient is a compound of Formula (I)

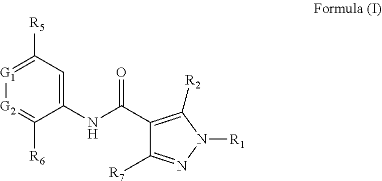

wherein

R₁ is independently selected from the group consisting of
i) naphthalen-1-yl, 4-amino-naphthalen-1-yl, 4-fluoronaphthalen-1-yl, or 5-fluoronaphthalen-1-yl;

and
ii) a heteroaryl selected from the group consisting of thieno[3,2-c]pyridin-4-yl, isoquinolin-4-yl, 8-fluoroquinolin-4-yl, furo[3,2-c]pyridin-4-yl, quinolin-5-yl, furo[2,3-c]pyridin-7-yl, benzofuran-4-yl 1,7-naphthyridin-5-yl, pyrrolo[1,2-a]pyrazin-1-yl, imidazo[1,2-a]pyridin-5-yl, 1-aminocarbonyl-isoquinolin-4-yl, pyrrolo[1,2-a]pyrazin-1-yl, benzo[d]thiazol-4-yl, 8-fluoro-1-hydroxyisoquinolin-4-yl, thieno[3,2-d]pyrimidin-4-yl, 8-fluoroimidazo[1,2-a]pyridin-5-yl, 3-methylimidazo[1,2-a]pyridin-5-yl, 1-aminoisoquinolin-4-yl, 1-oxo-quinolin-4-yl, 8-aminoquinolin-5-yl, benzo[d]oxazol-4-yl, 3-methylthieno[3,2-b]pyridin-7-yl, 1-(hydroxymethyl)isoquinolin-4-yl, (3R-hydroxypyrrolidin-1-yl)isoquinolin-4-yl, (1-hydroxyethyl)isoquinolin-4-yl, 8-fluoroisoquinolin-4-yl, 2-(difluoromethyl)quinolin-4-yl, 8-fluoroquinolin-5-yl, 1-hydroxyisoquinolin-4-yl, benzo[d]thiazol-4-yl, 1-aminoisoquinolin-4-yl, 1-(tetrahydrofuran-2-yl)isoquinolin-4-yl, 7-(difluoromethyl)thieno[2,3-c]pyridin-4-yl, 1-(1-hydroxyethyl)isoquinolin-4-yl, 1-cyanoisoquinolin-4-yl, 1-(1(R)-hydroxyethyl)isoquinolin-4-yl, quinazolin-4-yl, 2-methylimidazo[1,2-a]pyridin-3-yl, thiazolo[5,4-d]pyrimidin-7-yl, imidazo[1,2-a]pyridin-5-yl, benzo[d][1,2,3]thiadiazol-7-yl, 6-N-oxido-1H-pyrazol-1-yl)thieno[2,3-c]pyridin-4-yl, imidazo[1,2-a]pyridin-3-yl, furo[2,3-d]pyrimidin-4-yl, 2-fluoroquinolin-5-yl, isoquinolin-5-yl, benzo[d]isothiazol-3-yl, 7-methylpyrazolo[1,5-a]pyridin-4-yl, 1-oxo-1,2-dihydroisoquinolin-4-yl, 2-methyl-1-oxo-1,2-dihydroisoquinolin-4-yl, 1-(3-hydroxyazetidin-1-yl)isoquinolin-4-yl, 8-fluoro-1-(3-hydroxyazetidin-1-yl)isoquinolin-4-yl, (R)-8-fluoro-1-(3-hydroxypyrrolidin-1-yl)isoquinolin-4-yl, (S)-8-fluoro-1-(3-hydroxypyrrolidin-1-yl)isoquinolin-4-yl, 3-hydroxyazetidin-1-yl)thieno[2,3-c]pyridin-4-yl, 8-(3-hydroxyazetidin-1-yl)imidazo[1,2-a]pyridin-5-yl, 7-(3-hydroxyazetidin-1-yl)pyrazolo[1,5-a]pyridin-4-yl, 1-(3-hydroxyazetidin-1-yl)isoquinolin-5-yl and 1-(hydroxyethyl)quinolin-4-yl;

R₂ is trifluoromethyl;

G₁ is N or C(R₄);

G₂ is N or C(R₃); such that only one of G₁ and G₂ is N in any instance;

R₃ is independently selected from the group consisting of trifluoromethyl, cyano, and chloro;

R₄ is independently selected from the group consisting of 2H-1,2,3-triazol-2-yl, 4-carboxy-2H-1,2,3-triazol-2-yl, 4-(hydroxymethyl)-2H-1,2,3-triazol-2-yl, 4-methyl-2H-1,2,3-triazol-2-yl, oxazol-2-yl, 1H-imidazol-2-yl, 4-amino-2H-1,2,3-triazol-2-yl, 4-(hydroxymethyl)-1H-pyrazol-1-yl, 4-(hydroxymethyl)-2H-1,2,3-triazol-2-yl, 4-((dimethylamino)methyl)-2H-1,2,3-triazol-2-yl, 4-methoxycarbonyl-2H-1,2,3-triazol-2-yl, 4-aminocarbonyl-2H-1,2,3-triazol-2-yl, 1-methyl-1H-pyrazol-3-yl, and 1,3,4-oxadiazol-2-yl;

R₅ is hydrogen, chloro, or cyano;

R₆ is hydrogen or methyl;

R₇ is hydrogen;

or an enantiomer, diastereomer, solvate, or pharmaceutically acceptable salt form thereof.

Additional embodiments of the invention include pharmaceutical formulations as described herein, wherein the active pharmaceutical ingredient is a compound of Formula (I) selected from the group consisting of.
N-(2-cyanopyridin-4-yl)-1-(naphthalen-1-yl)-5-(trifluoromethyl)-1H-pyrazole-4-carboxamide;
N-(5-chloro-6-(2H-1,2,3-triazol-2-yl)pyridin-3-yl)-1-(naphthalen-1-yl)-5-(trifluoromethyl)-1H-pyrazole-4-carboxamide;
1-(naphthalen-1-yl)-5-(trifluoromethyl)-N-(2-(trifluoromethyl)pyridin-4-yl)-1H-pyrazole-4-carboxamide;
1-(naphthalen-1-yl)-5-(trifluoromethyl)-N-(5-(trifluoromethyl)pyridin-3-yl)-1H-pyrazole-4-carboxamide;
N-(5-cyanopyridin-3-yl)-1-(naphthalen-1-yl)-5-(trifluoromethyl)-1H-pyrazole-4-carboxamide;
1-(quinolin-5-yl)-5-(trifluoromethyl)-N-(2-(trifluoromethyl)pyridin-4-yl)-1H-pyrazole-4-carboxamide;
N-(5-chloro-6-methoxypyridin-3-yl)-1-(quinolin-5-yl)-5-(trifluoromethyl)-1H-pyrazole-4-carboxamide;
N-(5-chloro-6-(2H-1,2,3-triazol-2-yl)pyridin-3-yl)-1-(quinolin-5-yl)-5-(trifluoromethyl)-1H-pyrazole-4-carboxamide;
N-(5-chloro-6-(2H-1,2,3-triazol-2-yl)pyridin-3-yl)-1-(3-methylisoquinolin-1-yl)-5-(trifluoromethyl)-1H-pyrazole-4-carboxamide;
N-(3-chloro-4-methoxyphenyl)-1-(isoquinolin-8-yl)-5-(trifluoromethyl)-1H-pyrazole-4-carboxamide;
N-(3-chloro-4-(2H-1,2,3-triazol-2-yl)phenyl)-1-(isoquinolin-8-yl)-5-(trifluoromethyl)-1H-pyrazole-4-carboxamide;
N-(3-chloro-4-(1H-pyrazol-1-yl)phenyl)-1-(isoquinolin-8-yl)-5-(trifluoromethyl)-1H-pyrazole-4-carboxamide;
N-(6-cyano-5-(trifluoromethyl)pyridin-3-yl)-1-(isoquinolin-8-yl)-5-(trifluoromethyl)-1H-pyrazole-4-carboxamide;
N-(4-(2-aminopyrimidin-4-yl)-3-chlorophenyl)-1-(isoquinolin-8-yl)-5-(trifluoromethyl)-1H-pyrazole-4-carboxamide;
N-(5-chloro-6-(1H-pyrazol-1-yl)pyridin-3-yl)-1-(isoquinolin-8-yl)-5-(trifluoromethyl)-1H-pyrazole-4-carboxamide;
N-(5-chloro-6-(2H-1,2,3-triazol-2-yl)pyridin-3-yl)-5-isobutyl-1-(quinolin-5-yl)-1H-pyrazole-4-carboxamide;
N-(5-chloro-6-(2H-1,2,3-triazol-2-yl)pyridin-3-yl)-5-ethyl-1-(quinolin-5-yl)-1H-pyrazole-4-carboxamide;
N-(3-chloro-4-(1H-1,2,3-triazol-1-yl)phenyl)-1-(isoquinolin-8-yl)-5-(trifluoromethyl)-1H-pyrazole-4-carboxamide;
N-(5-cyano-6-(2H-1,2,3-triazol-2-yl)pyridin-3-yl)-1-(isoquinolin-8-yl)-5-(trifluoromethyl)-1H-pyrazole-4-carboxamide;
N-(5-chloro-6-(1,1-dioxidoisothiazolidin-2-yl)pyridin-3-yl)-1-(isoquinolin-8-yl)-5-(trifluoromethyl)-1H-pyrazole-4-carboxamide;
N-(5-chloro-6-(1-methyl-1H-pyrazol-3-yl)pyridin-3-yl)-1-(isoquinolin-8-yl)-5-(trifluoromethyl)-1H-pyrazole-4-carboxamide;
N-(5-chloro-6-(oxazol-2-yl)pyridin-3-yl)-1-(isoquinolin-8-yl)-5-(trifluoromethyl)-1H-pyrazole-4-carboxamide;
N-(5-chloro-6-methoxypyridin-3-yl)-1-(isoquinolin-4-yl)-5-(trifluoromethyl)-1H-pyrazole-4-carboxamide;
N-(5-cyano-6-methoxypyridin-3-yl)-1-(isoquinolin-4-yl)-5-(trifluoromethyl)-1H-pyrazole-4-carboxamide;
N-(5-chloro-6-(2H-1,2,3-triazol-2-yl)pyridin-3-yl)-1-(3-fluoroquinolin-5-yl)-5-(trifluoromethyl)-1H-pyrazole-4-carboxamide;
N-(5-chloro-6-(2H-1,2,3-triazol-2-yl)pyridin-3-yl)-5-isopropyl-1-(quinolin-5-yl)-1H-pyrazole-4-carboxamide;
N-(5-chloro-6-(2H-1,2,3-triazol-2-yl)pyridin-3-yl)-1-(6-methylquinolin-5-yl)-5-(trifluoromethyl)-1H-pyrazole-4-carboxamide;
N-(5-chloro-6-(2H-1,2,3-triazol-2-yl)pyridin-3-yl)-1-(8-methylquinolin-5-yl)-5-(trifluoromethyl)-1H-pyrazole-4-carboxamide;
N-(3-chloro-4-(3-methyl-1H-1,2,4-triazol-1-yl)phenyl)-1-(isoquinolin-8-yl)-5-(trifluoromethyl)-1H-pyrazole-4-carboxamide;
N-(5-chloro-6-(3-methyl-1H-1,2,4-triazol-1-yl)pyridin-3-yl)-1-(isoquinolin-8-yl)-5-(trifluoromethyl)-1H-pyrazole-4-carboxamide;
N-(3-chloro-4-(5-methyl-1H-1,2,4-triazol-1-yl)phenyl)-1-(isoquinolin-8-yl)-5-(trifluoromethyl)-1H-pyrazole-4-carboxamide;
N-(5-chloro-6-(2H-1,2,3-triazol-2-yl)pyridin-3-yl)-1-(isoquinolin-4-yl)-5-(trifluoromethyl)-1H-pyrazole-4-carboxamide;
N-(5-chloro-6-(2H-1,2,3-triazol-2-yl)pyridin-3-yl)-1-(4-methylisoquinolin-8-yl)-5-(trifluoromethyl)-1H-pyrazole-4-carboxamide;
1-(benzofuran-4-yl)-N-(5-chloro-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-5-(1-methoxyethyl)-1-(quinolin-5-yl)-1H-pyrazole-4-carboxamide;
N-(5-chloro-6-(2H-1,2,3-triazol-2-yl)pyridin-3-yl)-1-(6-methylisoquinolin-4-yl)-5-(trifluoromethyl)-1H-pyrazole-4-carboxamide;
N-(5-chloro-6-(2H-1,2,3-triazol-2-yl)pyridin-3-yl)-1-(2-methylquinolin-5-yl)-5-(trifluoromethyl)-1H-pyrazole-4-carboxamide;
N-(3-chloro-4-(2H-1,2,3-triazol-2-yl)phenyl)-1-(isoquinolin-4-yl)-5-(trifluoromethyl)-1H-pyrazole-4-carboxamide;
N-(5-chloro-6-(2H-1,2,3-triazol-2-yl)pyridin-3-yl)-5-methyl-1-(quinolin-5-yl)-1H-pyrazole-4-carboxamide;
N-(5-chloro-6-(2H-1,2,3-triazol-2-yl)pyridin-3-yl)-1-(8-fluoroquinolin-5-yl)-5-(trifluoromethyl)-1H-pyrazole-4-carboxamide;
N-(6-cyano-5-fluoropyridin-3-yl)-1-(isoquinolin-4-yl)-5-(trifluoromethyl)-1H-pyrazole-4-carboxamide;
N-(5-chloro-6-(1,1-dioxidoisothiazolidin-2-yl)pyridin-3-yl)-1-(isoquinolin-4-yl)-5-(trifluoromethyl)-1H-pyrazole-4-carboxamide;
N-(3-chloro-4-(1H-1,2,3-triazol-1-yl)phenyl)-1-(isoquinolin-4-yl)-5-(trifluoromethyl)-1H-pyrazole-4-carboxamide;
methyl 3-chloro-5-(3-chloro-5-(1-(isoquinolin-4-yl)-5-(trifluoromethyl)-1H-pyrazole-4-carboxamido)picolinamido)picolinate;
N-(5-chloro-6-((1-methylpiperidin-4-yl)oxy)pyridin-3-yl)-1-(isoquinolin-4-yl)-5-(trifluoromethyl)-1H-pyrazole-4-carboxamide;
N-(5-chloro-6-(1H-pyrazol-1-yl)pyridin-3-yl)-1-(isoquinolin-4-yl)-5-(trifluoromethyl)-1H-pyrazole-4-carboxamide;
N-(5-cyano-6-(2H-1,2,3-triazol-2-yl)pyridin-3-yl)-1-(isoquinolin-4-yl)-5-(trifluoromethyl)-1H-pyrazole-4-carboxamide;
N-(5-chloro-6-(4-methylpiperazine-1-carbonyl)pyridin-3-yl)-1-(isoquinolin-4-yl)-5-(trifluoromethyl)-1H-pyrazole-4-carboxamide;
N-(6-cyano-5-(trifluoromethyl)pyridin-3-yl)-1-(isoquinolin-4-yl)-5-(trifluoromethyl)-1H-pyrazole-4-carboxamide;
N-(5-chloro-6-(oxazol-2-yl)pyridin-3-yl)-1-(isoquinolin-4-yl)-5-(trifluoromethyl)-1H-pyrazole-4-carboxamide;
N-(3-chloro-4-(5-methyl-1H-1,2,4-triazol-1-yl)phenyl)-1-(isoquinolin-4-yl)-5-(trifluoromethyl)-1H-pyrazole-4-carboxamide;
N-(5-chloro-6-(1-methyl-1H-pyrazol-3-yl)pyridin-3-yl)-1-(isoquinolin-4-yl)-5-(trifluoromethyl)-1H-pyrazole-4-carboxamide;
N-(5-chloro-6-(3-methyl-1H-1,2,4-triazol-1-yl)pyridin-3-yl)-1-(isoquinolin-4-yl)-5-(trifluoromethyl)-1H-pyrazole-4-carboxamide;
N-(5-chloro-6-(5-methyl-1H-1,2,4-triazol-1-yl)pyridin-3-yl)-1-(isoquinolin-4-yl)-5-(trifluoromethyl)-1H-pyrazole-4-carboxamide;
N-(3-chloro-4-(3-methyl-1H-1,2,4-triazol-1-yl)phenyl)-1-(isoquinolin-4-yl)-5-(trifluoromethyl)-1H-pyrazole-4-carboxamide;
N-(5-chloro-6-(2H-1,2,3-triazol-2-yl)pyridin-3-yl)-5-(difluoromethyl)-1-(isoquinolin-1-yl)-1H-pyrazole-4-carboxamide;
N-(5-chloro-6-(2H-1,2,3-triazol-2-yl)pyridin-3-yl)-1-(isoquinolin-1-yl)-5-(trifluoromethyl)-1H-pyrazole-4-carboxamide;
N-(4-(2-aminopyrimidin-4-yl)-3-chlorophenyl)-1-(isoquinolin-4-yl)-5-(trifluoromethyl)-1H-pyrazole-4-carboxamide;
N-(3-cyano-4-(2H-1,2,3-triazol-2-yl)phenyl)-1-(isoquinolin-4-yl)-5-(trifluoromethyl)-1H-pyrazole-4-carboxamide;
N-(5-chloro-6-(2H-1,2,3-triazol-2-yl)pyridin-3-yl)-1-(quinolin-5-yl)-1H-pyrazole-4-carboxamide;
N-(5-fluoro-6-(2H-1,2,3-triazol-2-yl)pyridin-3-yl)-1-(isoquinolin-4-yl)-5-(trifluoromethyl)-1H-pyrazole-4-carboxamide;
N-(3-cyano-4-(1H-1,2,3-triazol-1-yl)phenyl)-1-(isoquinolin-4-yl)-5-(trifluoromethyl)-1H-pyrazole-4-carboxamide;
N-(5-chloro-6-(thiazol-2-yl)pyridin-3-yl)-1-(isoquinolin-4-yl)-5-(trifluoromethyl)-1H-pyrazole-4-carboxamide;
N-(5-chloro-6-(2H-1,2,3-triazol-2-yl)pyridin-3-yl)-5-methyl-1-(quinolin-4-yl)-1H-pyrazole-4-carboxamide;
N-(5-chloro-6-(2H-1,2,3-triazol-2-yl)pyridin-3-yl)-1-(3-methylquinolin-5-yl)-5-(trifluoromethyl)-1H-pyrazole-4-carboxamide;
N-(5-chloro-6-(2H-1,2,3-triazol-2-yl)pyridin-3-yl)-1-(1-methylisoquinolin-4-yl)-5-(trifluoromethyl)-1H-pyrazole-4-carboxamide;
N-(5-chloro-6-(2H-1,2,3-triazol-2-yl)pyridin-3-yl)-1-(6-fluoroquinolin-7-yl)-5-(trifluoromethyl)-1H-pyrazole-4-carboxamide;
N-(5-chloro-6-(2H-1,2,3-triazol-2-yl)pyridin-3-yl)-1-(1H-indazol-4-yl)-5-(trifluoromethyl)-1H-pyrazole-4-carboxamide;
N-(5-chloro-6-(1,3,4-oxadiazol-2-yl)pyridin-3-yl)-1-(isoquinolin-4-yl)-5-(trifluoromethyl)-1H-pyrazole-4-carboxamide;
N-(5-chloro-6-(1H-imidazol-1-yl)pyridin-3-yl)-1-(isoquinolin-4-yl)-5-(trifluoromethyl)-1H-pyrazole-4-carboxamide;
N-(6-(2H-1,2,3-triazol-2-yl)pyridin-3-yl)-1-(isoquinolin-4-yl)-5-(trifluoromethyl)-1H-pyrazole-4-carboxamide;
N-(4-aminobutyl)-3-chloro-5-(1-(isoquinolin-4-yl)-5-(trifluoromethyl)-1H-pyrazole-4-carboxamido)picolinamide;
1-(isoquinolin-4-yl)-N-(2-methyl-6-(trifluoromethyl)pyridin-4-yl)-5-(trifluoromethyl)-1H-pyrazole-4-carboxamide;
methyl 6-chloro-4-(1-(isoquinolin-4-yl)-5-(trifluoromethyl)-1H-pyrazole-4-carboxamido)picolinate;
methyl 4-(1-(isoquinolin-4-yl)-5-(trifluoromethyl)-1H-pyrazole-4-carboxamido)picolinate
N-(5-chloro-6-(2H-1,2,3-triazol-2-yl)pyridin-3-yl)-1-(isoquinolin-4-yl)-1H-pyrazole-4-carboxamide;
N-(2-cyanopyridin-4-yl)-1-(isoquinolin-4-yl)-5-(trifluoromethyl)-1H-pyrazole-4-carboxamide;
N-(5-cyano-6-(1-methyl-1H-pyrazol-3-yl)pyridin-3-yl)-1-(isoquinolin-4-yl)-5-(trifluoromethyl)-1H-pyrazole-4-carboxamide;
N-(5-chloro-6-cyclopropoxypyridin-3-yl)-1-(quinolin-5-yl)-5-(trifluoromethyl)-1H-pyrazole-4-carboxamide;
N-(5-cyano-6-((1-methylpiperidin-4-yl)oxy)pyridin-3-yl)-1-(isoquinolin-4-yl)-5-(trifluoromethyl)-1H-pyrazole-4-carboxamide;
N-(5-cyano-6-ethoxypyridin-3-yl)-1-(quinolin-5-yl)-5-(trifluoromethyl)-1H-pyrazole-4-carboxamide;
N-(5-cyanopyridin-3-yl)-1-(isoquinolin-4-yl)-5-(trifluoromethyl)-1H-pyrazole-4-carboxamide;
N-(5-cyano-6-(2H-1,2,3-triazol-2-yl)pyridin-3-yl)-1-(quinolin-5-yl)-5-(trifluoromethyl)-1H-pyrazole-4-carboxamide;
N-(6-(4-aminobutoxy)-5-cyanopyridin-3-yl)-1-(isoquinolin-4-yl)-5-(trifluoromethyl)-1H-pyrazole-4-carboxamide;
N-(5-cyano-6-methoxypyridin-3-yl)-1-(quinolin-5-yl)-5-(trifluoromethyl)-1H-pyrazole-4-carboxamide;
N-(5-cyano-6-(1H-1,2,4-triazol-1-yl)pyridin-3-yl)-1-(quinolin-5-yl)-5-(trifluoromethyl)-1H-pyrazole-4-carboxamide;
N-(8-chloro-3-oxo-3,4-dihydro-2H-benzo[b][1,4]oxazin-6-yl)-1-(isoquinolin-4-yl)-5-(trifluoromethyl)-1H-pyrazole-4-carboxamide;
N-(5-cyano-6-cyclopropoxypyridin-3-yl)-1-(quinolin-5-yl)-5-(trifluoromethyl)-1H-pyrazole-4-carboxamide;
N-(5-cyano-6-(1-methyl-1H-pyrazol-3-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(thieno[3,2-c]pyridin-4-yl)-5-(trifluoromethyl)-1H-pyrazole-4-carboxamide;
N-(5-chloro-6-(oxazol-2-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(8-fluoroquinolin-4-yl)-5-(trifluoromethyl)-1H-pyrazole-4-carboxamide;
1-(cinnolin-4-yl)-N-(5-cyano-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(furo[3,2-c]pyridin-4-yl)-5-(trifluoromethyl)-1H-pyrazole-4-carboxamide;
N-(8-chloro-4-methyl-3-oxo-3,4-dihydro-2H-benzo[b][1,4]oxazin-6-yl)-1-(quinolin-5-yl)-5-(trifluoromethyl)-1H-pyrazole-4-carboxamide;
N-(5-cyano-6-(4-methylpiperazine-1-carbonyl)pyridin-3-yl)-1-(isoquinolin-4-yl)-5-(trifluoromethyl)-1H-pyrazole-4-carboxamide;
N-(8-chloro-4-methyl-3-oxo-3,4-dihydro-2H-benzo[b][1,4]oxazin-6-yl)-1-(isoquinolin-4-yl)-5-(trifluoromethyl)-1H-pyrazole-4-carboxamide;
N-(5-chloro-6-(1-methyl-1H-imidazol-2-yl)pyridin-3-yl)-1-(isoquinolin-4-yl)-5-(trifluoromethyl)-1H-pyrazole-4-carboxamide;
N-(5-cyano-6-(2H-1,2,3-triazol-2-yl)pyridin-3-yl)-1-(furo[2,3-c]pyridin-7-yl)-5-(trifluoromethyl)-1H-pyrazole-4-carboxamide;
N-(5-cyano-6-(2H-1,2,3-triazol-2-yl)pyridin-3-yl)-1-(1,6-naphthyridin-5-yl)-5-(trifluoromethyl)-1H-pyrazole-4-carboxamide;
N-(6-(4-(4-aminobutyl)piperazine-1-carbonyl)-5-cyanopyridin-3-yl)-1-(isoquinolin-4-yl)-5-(trifluoromethyl)-1H-pyrazole-4-carboxamide;
N-(5-cyano-6-(2H-1,2,3-triazol-2-yl)pyridin-3-yl)-1-(phthalazin-1-yl)-5-(trifluoromethyl)-1H-pyrazole-4-carboxamide;
N-(5-cyano-6-(2H-1,2,3-triazol-2-yl)pyridin-3-yl)-1-(imidazo[1,2-a]pyrazin-8-yl)-5-(trifluoromethyl)-1H-pyrazole-4-carboxamide;
N-(5-chloro-6-(1H-imidazol-2-yl)pyridin-3-yl)-1-(isoquinolin-4-yl)-5-(trifluoromethyl)-1H-pyrazole-4-carboxamide;
N-(5-cyano-6-(2H-1,2,3-triazol-2-yl)pyridin-3-yl)-1-(quinoxalin-5-yl)-5-(trifluoromethyl)-1H-pyrazole-4-carboxamide;
N-(2-methyl-1-oxo-1,2,3,4-tetrahydroisoquinolin-7-yl)-1-(quinolin-5-yl)-5-(trifluoromethyl)-1H-pyrazole-4-carboxamide;
tert-butyl 2-(5-(4-((5-chloro-6-(2H-1,2,3-triazol-2-yl)pyridin-3-yl)carbamoyl)-5-(trifluoromethyl)-1H-pyrazol-1-yl)isoquinolin-1-yl)azetidine-1-carboxylate;
N-(3-(methylsulfonyl)-4-(1H-1,2,3-triazol-1-yl)phenyl)-1-(quinolin-5-yl)-5-(trifluoromethyl)-1H-pyrazole-4-carboxamide;
1-(1,5-bis(tetrahydrofuran-2-yl)isoquinolin-4-yl)-N-(5-chloro-6-(2H-1,2,3-triazol-2-yl)pyridin-3-yl)-5-(trifluoromethyl)-1H-pyrazole-4-carboxamide;
N-(4-methyl-3-oxo-3,4-dihydro-2H-benzo[b][1,4]oxazin-6-yl)-1-(quinolin-5-yl)-5-(trifluoromethyl)-1H-pyrazole-4-carboxamide;
1-(1-(azetidin-2-yl)isoquinolin-5-yl)-N-(5-chloro-6-(2H-1,2,3-triazol-2-yl)pyridin-3-yl)-5-(trifluoromethyl)-1H-pyrazole-4-carboxamide;
N-(3-(methylsulfonyl)-4-(2H-1,2,3-triazol-2-yl)phenyl)-1-(quinolin-5-yl)-5-(trifluoromethyl)-1H-pyrazole-4-carboxamide;
N-(2-methyl-1-oxo-1,2-dihydroisoquinolin-7-yl)-1-(quinolin-5-yl)-5-(trifluoromethyl)-1H-pyrazole-4-carboxamide;
1-(2-(azetidin-2-yl)quinolin-5-yl)-N-(5-chloro-6-(2H-1,2,3-triazol-2-yl)pyridin-3-yl)-5-(trifluoromethyl)-1H-pyrazole-4-carboxamide;
N-(3-oxo-3,4-dihydro-2H-benzo[b][1,4]oxazin-6-yl)-1-(quinolin-5-yl)-5-(trifluoromethyl)-1H-pyrazole-4-carboxamide;
1-(benzo[d]thiazol-4-yl)-N-(2,5-dimethyl-6-(2H-1,2,3-triazol-2-yl)pyridin-3-yl)-5-(trifluoromethyl)-1H-pyrazole-4-carboxamide;
N-(5-methyl-6-(3-methyl-2-oxo-2,3-dihydro-1H-imidazol-1-yl)pyridin-3-yl)-1-(quinolin-5-yl)-5-(trifluoromethyl)-1H-pyrazole-4-carboxamide;
N-(2,5-diethyl-6-(2H-1,2,3-triazol-2-yl)pyridin-3-yl)-1-(imidazo[1,2-a]pyridin-5-yl)-5-(trifluoromethyl)-1H-pyrazole-4-carboxamide;
N-(5-chloro-6-(2H-1,2,3-triazol-2-yl)pyridin-3-yl)-1-(2-(difluoromethyl)quinolin-5-yl)-5-(trifluoromethyl)-1H-pyrazole-4-carboxamide;
N-(5-cyano-6-(2H-1,2,3-triazol-2-yl)pyridin-3-yl)-1-(2-methylbenzo[d]oxazol-4-yl)-5-(trifluoromethyl)-1H-pyrazole-4-carboxamide;
N-(5-chloro-6-(2H-1,2,3-triazol-2-yl)pyridin-3-yl)-1-(2-methoxyquinolin-5-yl)-5-(trifluoromethyl)-1H-pyrazole-4-carboxamide;
1-(1-aminoisoquinolin-5-yl)-N-(5-cyano-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(2-cyanoquinolin-5-yl)-5-(trifluoromethyl)-1H-pyrazole-4-carboxamide;
1-(2-methylimidazo[1,2-a]pyridin-5-yl)-5-(trifluoromethyl)-N-(2-(trifluoromethyl)pyridin-4-yl)-1H-pyrazole-4-carboxamide;
1-(2-chloroquinolin-4-yl)-N-(5-cyano-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(2-chloroquinolin-5-yl)-5-(trifluoromethyl)-1H-pyrazole-4-carboxamide;
N-(5-chloro-6-(2H-1,2,3-triazol-2-yl)pyridin-3-yl)-1-(2-(tetrahydrofuran-2-yl)quinolin-5-yl)-5-(trifluoromethyl)-1H-pyrazole-4-carboxamide;
5-(4-((5-chloro-6-(2H-1,2,3-triazol-2-yl)pyridin-3-yl)carbamoyl)-5-(trifluoromethyl)-1H-pyrazol-1-yl)quinoline-2-carboxamide;
N-(5-chloro-6-(2H-1,2,3-triazol-2-yl)pyridin-3-yl)-1-(1H-pyrazolo[3,4-d]pyrimidin-4-yl)-5-(trifluoromethyl)-1H-pyrazole-4-carboxamide;
N-(5-cyano-6-(2H-1,2,3-triazol-2-yl)pyridin-3-yl)-1-(1,6-naphthyridin-4-yl)-5-(trifluoromethyl)-1H-pyrazole-4-carboxamide;
N-(5-cyano-6-(4-methylpiperazin-1-yl)pyridin-3-yl)-1-(quinolin-5-yl)-5-(trifluoromethyl)-1H-pyrazole-4-carboxamide;
(*R)—N-(5-chloro-6-(2H-1,2,3-triazol-2-yl)pyridin-3-yl)-1-(1-(1-hydroxyethyl)isoquinolin-5-yl)-5-(trifluoromethyl)-1H-pyrazole-4-carboxamide;
1-(benzo[d]thiazol-4-yl)-N-(5-cyano-2-methyl-4-(2H-1,2,3-triazol-2-yl)phenyl)-5-(trifluoromethyl)-1H-pyrazole-4-carboxamide;
(*R)—N-(5-chloro-6-(2H-1,2,3-triazol-2-yl)pyridin-3-yl)-1-(2-(tetrahydrofuran-2-yl)quinolin-5-yl)-5-(trifluoromethyl)-1H-pyrazole-4-carboxamide;
N-(5-chloro-6-(2-oxopyrrolidin-1-yl)pyridin-3-yl)-1-(isoquinolin-4-yl)-5-(trifluoromethyl)-1H-pyrazole-4-carboxamide;
N-(1-methyl-1H-pyrazolo[3,4-b]pyridin-5-yl)-1-(quinolin-5-yl)-5-(trifluoromethyl)-1H-pyrazole-4-carboxamide;
N-(5-chloro-6-(2H-1,2,3-triazol-2-yl)pyridin-3-yl)-1-(2-methyl-1-oxo-1,2-dihydroisoquinolin-5-yl)-5-(trifluoromethyl)-1H-pyrazole-4-carboxamide;
N-(5-chloro-6-(5-cyano-1H-1,2,3-triazol-1-yl)pyridin-3-yl)-1-(quinolin-5-yl)-5-(trifluoromethyl)-1H-pyrazole-4-carboxamide;
2-(2-chloro-4-(1-(quinolin-5-yl)-5-(trifluoromethyl)-1H-pyrazole-4-carboxamido)phenyl)-2H-1,2,3-triazole-4-carboxylic acid;
N-(1H-pyrazolo[3,4-b]pyridin-5-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(imidazo[1,2-a]pyridin-8-yl)-5-(trifluoromethyl)-1H-pyrazole-4-carboxamide;
(*S)—N-(5-chloro-6-(2H-1,2,3-triazol-2-yl)pyridin-3-yl)-1-(1-(1-hydroxyethyl)isoquinolin-5-yl)-5-(trifluoromethyl)-1H-pyrazole-4-carboxamide;
N-(2-methylpyridin-4-yl)-1-(1-oxo-1,2-dihydroisoquinolin-5-yl)-5-(trifluoromethyl)-1H-pyrazole-4-carboxamide;
1-(benzo[d][1,2,3]thiadiazol-4-yl)-N-(5-chloro-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(2-methylthieno[3,2-b]pyridin-7-yl)-5-(trifluoromethyl)-1H-pyrazole-4-carboxamide;
1-(1-(azetidin-3-yl)isoquinolin-4-yl)-N-(5-chloro-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(imidazo[1,5-a]pyridin-5-yl)-5-(trifluoromethyl)-1H-pyrazole-4-carboxamide;
1-(3-chloro-5-(1-(quinolin-5-yl)-5-(trifluoromethyl)-1H-pyrazole-4-carboxamido)pyridin-2-yl)-1H-1,2,3-triazole-4-carboxylic acid;
N-(5-methoxy-6-(1H-1,2,3-triazol-1-yl)pyridin-3-yl)-1-(quinolin-5-yl)-5-(trifluoromethyl)-1H-pyrazole-4-carboxamide;
N-(5-chloro-6-(2H-1,2,3-triazol-2-yl)pyridin-3-yl)-1-(2-oxo-1,2-dihydroquinolin-5-yl)-5-(trifluoromethyl)-1H-pyrazole-4-carboxamide;
N-(4-aminobutyl)-3-cyano-5-(1-(isoquinolin-4-yl)-5-(trifluoromethyl)-1H-pyrazole-4-carboxamido)picolinamide;
2-cyano-4-(1-(quinolin-5-yl)-5-(trifluoromethyl)-1H-pyrazole-4-carboxamido)benzoic acid
N-(4-(4-(aminomethyl)-1H-pyrazol-1-yl)-3-methylphenyl)-1-(quinolin-5-yl)-5-(trifluoromethyl)-1H-pyrazole-4-carboxamide;
1-(2-aminoquinolin-4-yl)-N-(5-cyano-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(1-methyl-1H-indazol-4-yl)-5-(trifluoromethyl)-1H-pyrazole-4-carboxamide;
1-(1-aminoisoquinolin-5-yl)-N-(5-chloro-6-(2H-1,2,3-triazol-2-yl)pyridin-3-yl)-5-(trifluoromethyl)-1H-pyrazole-4-carboxamide;
N-(5-methyl-6-(1-methyl-1H-tetrazol-5-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(1-methyl-1H-pyrazolo[3,4-b]pyridin-4-yl)-5-(trifluoromethyl)-1H-pyrazole-4-carboxamide;
N-(5-chloro-6-(2H-1,2,3-triazol-2-yl)pyridin-3-yl)-1-(1-ethoxyisoquinolin-4-yl)-5-(trifluoromethyl)-1H-pyrazole-4-carboxamide;
1-(1-(azetidin-2-yl)isoquinolin-4-yl)-N-(5-chloro-6-(2H-1,2,3-triazol-2-yl)pyridin-3-yl)-5-(trifluoromethyl)-1H-pyrazole-4-carboxamide;
1-(2-aminoquinolin-5-yl)-N-(5-chloro-6-(2H-1,2,3-triazol-2-yl)pyridin-3-yl)-5-(trifluoromethyl)-1H-pyrazole-4-carboxamide;
1-(1-acetylisoquinolin-5-yl)-N-(5-chloro-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(4-(difluoromethyl)quinolin-5-yl)-5-(trifluoromethyl)-1H-pyrazole-4-carboxamide;
N-(5-chloro-6-(2H-1,2,3-triazol-2-yl)pyridin-3-yl)-1-(1-(pyrrolidin-2-yl)isoquinolin-4-yl)-5-(trifluoromethyl)-1H-pyrazole-4-carboxamide;
N-(5-chloro-6-(difluoromethoxy)pyridin-3-yl)-1-(isoquinolin-4-yl)-5-(trifluoromethyl)-1H-pyrazole-4-carboxamide;
N-(5-chloro-6-(2H-1,2,3-triazol-2-yl)pyridin-3-yl)-1-(imidazo[1,2-b]pyridazin-6-yl)-5-(trifluoromethyl)-1H-pyrazole-4-carboxamide;
1-(1-aminoisoquinolin-4-yl)-N-(2,5-dimethyl-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(1-methoxyisoquinolin-5-yl)-5-(trifluoromethyl)-1H-pyrazole-4-carboxamide;
1-(2-aminoquinolin-5-yl)-N-(5-cyano-6-(2H-1,2,3-triazol-2-yl)pyridin-3-yl)-5-(trifluoromethyl)-1H-pyrazole-4-carboxamide;
2-(2-chloro-4-(1-(quinolin-5-yl)-5-(trifluoromethyl)-1H-pyrazole-4-carboxamido)phenyl)-2H-1,2,3-triazole-4-carboxamide;
N-(5-chloro-6-(2H-1,2,3-triazol-2-yl)pyridin-3-yl)-1-(1-(methylthio)isoquinolin-4-yl)-5-(trifluoromethyl)-1H-pyrazole-4-carboxamide;
N-(5-chloro-6-(2H-1,2,3-triazol-2-yl)pyridin-3-yl)-3-fluoro-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(2-methylbenzo[d]thiazol-4-yl)-5-(trifluoromethyl)-1H-pyrazole-4-carboxamide;
1-(3-chloro-5-(1-(quinolin-5-yl)-5-(trifluoromethyl)-1H-pyrazole-4-carboxamido)pyridin-2-yl)-1H-1,2,3-triazole-4-carboxamide;
N-(5-chloro-6-(2H-1,2,3-triazol-2-yl)pyridin-3-yl)-5-cyano-1-(quinolin-5-yl)-1H-pyrazole-4-carboxamide;
1-(7-methylpyrazolo[1,5-a]pyridin-4-yl)-5-(trifluoromethyl)-N-(2-(trifluoromethyl)pyridin-4-yl)-1H-pyrazole-4-carboxamide;
N-(6-(2H-[1,2,3]triazolo[4,5-c]pyridin-2-yl)-5-chloropyridin-3-yl)-1-(quinolin-5-yl)-5-(trifluoromethyl)-1H-pyrazole-4-carboxamide;
1-(2-acetylquinolin-4-yl)-N-(5-chloro-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(2-methylbenzo[d]thiazol-7-yl)-5-(trifluoromethyl)-1H-pyrazole-4-carboxamide;
N-(6-(5-(aminomethyl)-1H-1,2,3-triazol-1-yl)-5-chloropyridin-3-yl)-1-(quinolin-5-yl)-5-(trifluoromethyl)-1H-pyrazole-4-carboxamide;
1-(2-(azetidin-2-yl)quinolin-4-yl)-N-(5-chloro-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(1-(1-ethoxyethyl)isoquinolin-4-yl)-5-(trifluoromethyl)-1H-pyrazole-4-carboxamide;
methyl 1-(3-chloro-5-(1-(quinolin-5-yl)-5-(trifluoromethyl)-1H-pyrazole-4-carboxamido)pyridin-2-yl)-1H-1,2,3-triazole-4-carboxylate
1-(imidazo[1,2-a]pyridin-5-yl)-5-(trifluoromethyl)-N-(2-(trifluoromethyl)pyridin-4-yl)-1H-pyrazole-4-carboxamide;
N-(5-chloro-2-ethyl-6-(2H-1,2,3-triazol-2-yl)pyridin-3-yl)-1-(imidazo[1,2-a]pyridin-5-yl)-5-(trifluoromethyl)-1H-pyrazole-4-carboxamide;
N-(5-chloro-6-(2H-1,2,3-triazol-2-yl)pyridin-3-yl)-1-(2-(difluoromethyl)thieno[2,3-c]pyridin-4-yl)-5-(trifluoromethyl)-1H-pyrazole-4-carboxamide;
N-(5-cyano-6-(2H-1,2,3-triazol-2-yl)pyridin-3-yl)-1-(imidazo[1,2-a]pyrimidin-5-yl)-5-(trifluoromethyl)-1H-pyrazole-4-carboxamide;
N-(5-methoxy-6-(2H-1,2,3-triazol-2-yl)pyridin-3-yl)-1-(quinolin-5-yl)-5-(trifluoromethyl)-1H-pyrazole-4-carboxamide;
N-(5-chloro-6-(2H-1,2,3-triazol-2-yl)pyridin-3-yl)-1-(2-methoxyquinolin-4-yl)-5-(trifluoromethyl)-1H-pyrazole-4-carboxamide;
1-(benzo[d][1,2,3]thiadiazol-7-yl)-N-(5-chloro-2-methyl-6-(1H-pyrazol-1-yl)pyridin-3-yl)-5-(trifluoromethyl)-1H-pyrazole-4-carboxamide;
N-(2,5-dimethyl-6-(2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
1-(benzo[d]thiazol-4-yl)-N-(5-chloro-2-methyl-6-(2H-1,2,3-triazol-2-yl)pyridin-3-yl)-5-(trifluoromethyl)-1H-pyrazole-4-carboxamide;
N-(5-chloro-6-(5-(methoxymethyl)-1H-1,2,3-triazol-1-yl)pyridin-3-yl)-1-(quinolin-5-yl)-5-(trifluoromethyl)-1H-pyrazole-4-carboxamide;
N-(6-(2H-[1,2,3]triazolo[4,5-b]pyridin-2-yl)-5-chloropyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(2-methyl-[1,2,4]triazolo[1,5-a]pyridin-5-yl)-5-(trifluoromethyl)-1H-pyrazole-4-carboxamide;
3-(3-cyano-5-(1-(quinolin-5-yl)-5-(trifluoromethyl)-1H-pyrazole-4-carboxamido)pyridin-2-yl)-1-methyl-1H-pyrazole-5-carboxylic acid;
1-(benzo[d]thiazol-4-yl)-N-(5-cyano-2-methyl-6-(2H-1,2,3-triazol-2-yl)pyridin-3-yl)-5-(trifluoromethyl)-1H-pyrazole-4-carboxamide;
1-(1-aminoisoquinolin-4-yl)-N-(5-chloro-2-methyl-6-(1-methyl-1H-pyrazol-3-yl)pyridin-3-yl)-5-(trifluoromethyl)-1H-pyrazole-4-carboxamide;
N-(6-(3H-[1,2,3]triazolo[4,5-b]pyridin-3-yl)-5-chloropyridin-3-yl)-1-(quinolin-5-yl)-5-(trifluoromethyl)-1H-pyrazole-4-carboxamide;
methyl 2-(2-chloro-4-(1-(quinolin-5-yl)-5-(trifluoromethyl)-1H-pyrazole-4-carboxamido)phenyl)-2H-1,2,3-triazole-4-carboxylate;
N-(5-cyano-6-(2-methyl-2H-1,2,3-triazol-4-yl)pyridin-3-yl)-1-(quinolin-5-yl)-5-(trifluoromethyl)-1H-pyrazole-4-carboxamide;
N-(3-chloro-4-(5-oxo-4,5-dihydro-1H-1,2,4-triazol-3-yl)phenyl)-1-(quinolin-5-yl)-5-(trifluoromethyl)-1H-pyrazole-4-carboxamide;
methyl 3-chloro-5-(1-(quinolin-5-yl)-5-(trifluoromethyl)-1H-pyrazole-4-carboxamido)picolinate;
N-(6-(1H-[1,2,3]triazolo[4,5-c]pyridin-1-yl)-5-chloropyridin-3-yl)-1-(quinolin-5-yl)-5-(trifluoromethyl)-1H-pyrazole-4-carboxamide;
N-(5-cyanopyridin-3-yl)-1-(1-oxo-1,2-dihydroisoquinolin-5-yl)-5-(trifluoromethyl)-1H-pyrazole-4-carboxamide;
N-(5-cyano-6-(2H-1,2,3-triazol-2-yl)pyridin-3-yl)-1-(1-methyl-1H-pyrazolo[3,4-c]pyridin-7-yl)-5-(trifluoromethyl)-1H-pyrazole-4-carboxamide;
1-(1-aminoisoquinolin-4-yl)-N-(5-chloro-2-methyl-6-(1H-pyrazol-1-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(1-(dimethylamino)isoquinolin-4-yl)-5-(trifluoromethyl)-1H-pyrazole-4-carboxamide;
N-(5-chloro-6-(2H-1,2,3-triazol-2-yl)pyridin-3-yl)-1-(8-(difluoromethyl)quinolin-5-yl)-5-(trifluoromethyl)-1H-pyrazole-4-carboxamide;
(*R)—N-(5-chloro-6-(2H-1,2,3-triazol-2-yl)pyridin-3-yl)-1-(1-(4-oxotetrahydrofuran-2-yl)isoquinolin-4-yl)-5-(trifluoromethyl)-1H-pyrazole-4-carboxamide;
N-(5-cyano-6-(2H-1,2,3-triazol-2-yl)pyridin-3-yl)-1-(2-methylfuro[3,2-b]pyridin-7-yl)-5-(trifluoromethyl)-1H-pyrazole-4-carboxamide;
N-(5-chloro-6-(2H-1,2,3-triazol-2-yl)pyridin-3-yl)-1-(1-(difluoromethyl)isoquinolin-5-yl)-5-(trifluoromethyl)-1H-pyrazole-4-carboxamide;
N-(5-cyano-6-(2H-1,2,3-triazol-2-yl)pyridin-3-yl)-1-(1-methyl-1H-indazol-7-yl)-5-(trifluoromethyl)-1H-pyrazole-4-carboxamide;
N-(5-chloro-6-(2H-1,2,3-triazol-2-yl)pyridin-3-yl)-1-(6-fluoroimidazo[1,2-a]pyridin-5-yl)-5-(trifluoromethyl)-1H-pyrazole-4-carboxamide;
N-(5-chloro-6-(2H-1,2,3-triazol-2-yl)pyridin-3-yl)-1-(5-fluoroquinolin-8-yl)-5-(trifluoromethyl)-1H-pyrazole-4-carboxamide;
N-(5-cyano-6-(2H-1,2,3-triazol-2-yl)pyridin-3-yl)-1-(1H-pyrazolo[4,3-b]pyridin-7-yl)-5-(trifluoromethyl)-1H-pyrazole-4-carboxamide;
N-(5-cyano-6-(2H-1,2,3-triazol-2-yl)pyridin-3-yl)-1-(4-fluoroisoquinolin-1-yl)-5-(trifluoromethyl)-1H-pyrazole-4-carboxamide;
5-(4-((5-chloro-6-(2H-1,2,3-triazol-2-yl)pyridin-3-yl)carbamoyl)-5-(trifluoromethyl)-1H-pyrazol-1-yl)isoquinoline-1-carboxamide;
N-(5-chloro-6-(2H-1,2,3-triazol-2-yl)pyridin-3-yl)-1-(1-(methylamino)isoquinolin-4-yl)-5-(trifluoromethyl)-1H-pyrazole-4-carboxamide;
(*S)—N-(5-chloro-6-(2H-1,2,3-triazol-2-yl)pyridin-3-yl)-1-(1-(1-hydroxyethyl)isoquinolin-4-yl)-5-(trifluoromethyl)-1H-pyrazole-4-carboxamide;
N-(5-chloro-6-(2H-1,2,3-triazol-2-yl)pyridin-3-yl)-1-(1-(1,1-difluoroethyl)isoquinolin-4-yl)-5-(trifluoromethyl)-1H-pyrazole-4-carboxamide;
5-chloro-N-(5-chloro-6-(2H-1,2,3-triazol-2-yl)pyridin-3-yl)-1-(quinolin-5-yl)-1H-pyrazole-4-carboxamide;
1-(1-aminoisoquinolin-4-yl)-N-(5-chloro-2-methyl-4-(2H-1,2,3-triazol-2-yl)phenyl)-5-(trifluoromethyl)-1H-pyrazole-4-carboxamide;
N-(5-chloro-6-(2H-1,2,3-triazol-2-yl)pyridin-3-yl)-1-(1-methoxyisoquinolin-4-yl)-5-(trifluoromethyl)-1H-pyrazole-4-carboxamide;
(*S)—N-(5-chloro-6-(2H-1,2,3-triazol-2-yl)pyridin-3-yl)-1-(1-(4-oxotetrahydrofuran-2-yl)isoquinolin-4-yl)-5-(trifluoromethyl)-1H-pyrazole-4-carboxamide;
(*S)—N-(5-chloro-6-(2H-1,2,3-triazol-2-yl)pyridin-3-yl)-1-(1-(tetrahydrofuran-2-yl)isoquinolin-4-yl)-5-(trifluoromethyl)-1H-pyrazole-4-carboxamide;
N-(5-chloro-6-(2H-1,2,3-triazol-2-yl)pyridin-3-yl)-1-(1-fluoroisoquinolin-4-yl)-5-(trifluoromethyl)-1H-pyrazole-4-carboxamide;
N-(5-cyano-6-(2H-1,2,3-triazol-2-yl)pyridin-3-yl)-1-(1-methyl-1H-pyrazolo[3,4-b]pyridin-3-yl)-5-(trifluoromethyl)-1H-pyrazole-4-carboxamide;
N-(5-chloro-2-methyl-6-(2H-1,2,3-triazol-2-yl)pyridin-3-yl)-1-(8-fluoroimidazo[1,2-a]pyridin-5-yl)-5-(trifluoromethyl)-1H-pyrazole-4-carboxamide;
N-(5-cyano-6-(2H-1,2,3-triazol-2-yl)pyridin-3-yl)-1-(1-methyl-1H-indazol-3-yl)-5-(trifluoromethyl)-1H-pyrazole-4-carboxamide;
N-(5-chloro-6-(2H-1,2,3-triazol-2-yl)pyridin-3-yl)-1-(1-(5-oxopyrrolidin-2-yl)isoquinolin-4-yl)-5-(trifluoromethyl)-1H-pyrazole-4-carboxamide;
methyl 3-(3-cyano-5-(1-(quinolin-5-yl)-5-(trifluoromethyl)-1H-pyrazole-4-carboxamido)pyridin-2-yl)-1-methyl-1H-pyrazole-5-carboxylate;
N-(5-cyano-6-(1-methyl-1H-pyrazol-3-yl)pyridin-3-yl)-1-(8-fluoroquinolin-5-yl)-5-(trifluoromethyl)-1H-pyrazole-4-carboxamide;
N-(5-cyano-6-(2H-1,2,3-triazol-2-yl)pyridin-3-yl)-1-(1,5-naphthyridin-4-yl)-5-(trifluoromethyl)-1H-pyrazole-4-carboxamide;
N-(5-chloro-6-(5-((dimethylamino)methyl)-1H-1,2,3-triazol-1-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(2-methylbenzo[d]oxazol-7-yl)-5-(trifluoromethyl)-1H-pyrazole-4-carboxamide;
N-(6-(4-(aminomethyl)-2H-1,2,3-triazol-2-yl)-5-chloropyridin-3-yl)-1-(quinolin-5-yl)-5-(trifluoromethyl)-1H-pyrazole-4-carboxamide;
N-(5-chloro-6-(1-methyl-1H-pyrazol-3-yl)pyridin-3-yl)-1-(8-fluoroquinolin-5-yl)-5-(trifluoromethyl)-1H-pyrazole-4-carboxamide;
1-(1-aminoisoquinolin-4-yl)-N-(5-cyano-2-methyl-4-(2H-1,2,3-triazol-2-yl)phenyl)-5-(trifluoromethyl)-1H-pyrazole-4-carboxamide;
1-(benzo[d][1,2,3]thiadiazol-7-yl)-N-(5-chloro-2-fluoro-4-(2H-1,2,3-triazol-2-yl)phenyl)-5-(trifluoromethyl)-1H-pyrazole-4-carboxamide;
1-([1,2,4]triazolo[1,5-a]pyridin-5-yl)-N-(5-cyano-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(1-((N-methylformamido)methyl)isoquinolin-4-yl)-5-(trifluoromethyl)-1H-pyrazole-4-carboxamide;
N-(5-cyano-6-(2H-1,2,3-triazol-2-yl)pyridin-3-yl)-1-(imidazo[1,2-a]pyrazin-5-yl)-5-(trifluoromethyl)-1H-pyrazole-4-carboxamide;
N-(5-cyano-6-(2H-1,2,3-triazol-2-yl)pyridin-3-yl)-1-(1,7-naphthyridin-4-yl)-5-(trifluoromethyl)-1H-pyrazole-4-carboxamide;
N-(5-chloro-2-methyl-6-(1H-pyrazol-1-yl)pyridin-3-yl)-1-(1-oxo-1,2-dihydroisoquinolin-5-yl)-5-(trifluoromethyl)-1H-pyrazole-4-carboxamide;
N-(5-chloro-6-(2H-1,2,3-triazol-2-yl)pyridin-3-yl)-1-(2-fluoroquinolin-5-yl)-5-(trifluoromethyl)-1H-pyrazole-4-carboxamide;
1-(2-aminobenzo[d]thiazol-7-yl)-N-(5-chloro-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(isothiazolo[5,4-b]pyridin-3-yl)-5-(trifluoromethyl)-1H-pyrazole-4-carboxamide;
N-(5-cyano-6-(1H-pyrrol-1-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(1-methoxyisoquinolin-5-yl)-5-(trifluoromethyl)-1H-pyrazole-4-carboxamide;
1-(2-aminobenzo[d]thiazol-7-yl)-N-(5-cyano-6-(2H-1,2,3-triazol-2-yl)pyridin-3-yl)-5-(trifluoromethyl)-1H-pyrazole-4-carboxamide;
N-(6-(1H-1,2,3-triazol-1-yl)-5-(trifluoromethyl)pyridin-3-yl)-1-(quinolin-5-yl)-5-(trifluoromethyl)-1H-pyrazole-4-carboxamide;
1-(benzo[d]isoxazol-3-yl)-N-(5-chloro-6-(2H-1,2,3-triazol-2-yl)pyridin-3-yl)-5-(trifluoromethyl)-1H-pyrazole-4-carboxamide;
1-(1-oxo-1,2-dihydroisoquinolin-5-yl)-5-(trifluoromethyl)-N-(2-(trifluoromethyl)pyridin-4-yl)-1H-pyrazole-4-carboxamide;
N-(5-chloro-6-(2H-1,2,3-triazol-2-yl)pyridin-3-yl)-1-(8-fluoro-1-(methylamino)isoquinolin-4-yl)-5-(trifluoromethyl)-1H-pyrazole-4-carboxamide;
5-bromo-N-(5-chloro-6-(2H-1,2,3-triazol-2-yl)pyridin-3-yl)-1-(quinolin-5-yl)-1H-pyrazole-4-carboxamide;
N-(5-chloro-6-(2H-1,2,3-triazol-2-yl)pyridin-3-yl)-1-(2-cyanoquinolin-4-yl)-5-(trifluoromethyl)-1H-pyrazole-4-carboxamide;
N-(5-chloro-6-(2H-1,2,3-triazol-2-yl)pyridin-3-yl)-1-(1-chloroisoquinolin-4-yl)-5-(trifluoromethyl)-1H-pyrazole-4-carboxamide;
1-(pyrazolo[1,5-a]pyridin-4-yl)-5-(trifluoromethyl)-N-(2-(trifluoromethyl)pyridin-4-yl)-1H-pyrazole-4-carboxamide;
N-(5-chloro-6-(2H-1,2,3-triazol-2-yl)pyridin-3-yl)-1-(2-methylimidazo[1,2-a]pyridin-5-yl)-5-(trifluoromethyl)-1H-pyrazole-4-carboxamide;
1-(1-oxo-1,2-dihydroisoquinolin-5-yl)-5-(trifluoromethyl)-N-(5-(trifluoromethyl)pyridin-3-yl)-1H-pyrazole-4-carboxamide;
1-(1-(1,4-dioxan-2-yl)isoquinolin-4-yl)-N-(5-chloro-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(1H-indazol-7-yl)-5-(trifluoromethyl)-1H-pyrazole-4-carboxamide;
4-(4-((5-chloro-6-(2H-1,2,3-triazol-2-yl)pyridin-3-yl)carbamoyl)-5-(trifluoromethyl)-1H-pyrazol-1-yl)quinoline-2-carboxamide;
N-(5-chloro-6-(5-(hydroxymethyl)-1H-1,2,3-triazol-1-yl)pyridin-3-yl)-1-(1-oxo-1,2-dihydroisoquinolin-5-yl)-5-(trifluoromethyl)-1H-pyrazole-4-carboxamide;
N-(5-chloro-6-(2H-1,2,3-triazol-2-yl)pyridin-3-yl)-1-(1-(4-oxotetrahydrofuran-2-yl)isoquinolin-4-yl)-5-(trifluoromethyl)-1H-pyrazole-4-carboxamide;
N-(6-(4-amino-2H-1,2,3-triazol-2-yl)-5-chloropyridin-3-yl)-1-(benzo[d]thiazol-4-yl)-5-(trifluoromethyl)-1H-pyrazole-4-carboxamide;
1-(2-amino-[1,2,4]triazolo[1,5-a]pyridin-5-yl)-N-(5-cyano-6-(2H-1,2,3-triazol-2-yl)pyridin-3-yl)-5-(trifluoromethyl)-1H-pyrazole-4-carboxamide;
(*R)—N-(5-chloro-6-(2H-1,2,3-triazol-2-yl)pyridin-3-yl)-1-(1-(tetrahydrofuran-2-yl)isoquinolin-4-yl)-5-(trifluoromethyl)-1H-pyrazole-4-carboxamide;
N-(5-bromo-6-(1H-1,2,3-triazol-1-yl)pyridin-3-yl)-1-(quinolin-5-yl)-5-(trifluoromethyl)-1H-pyrazole-4-carboxamide;
N-(5-chloro-6-(oxazol-2-yl)pyridin-3-yl)-1-(8-fluoroquinolin-5-yl)-5-(trifluoromethyl)-1H-pyrazole-4-carboxamide;
N-(5-chloro-6-methoxypyridin-3-yl)-1-(1-oxo-1,2-dihydroisoquinolin-5-yl)-5-(trifluoromethyl)-1H-pyrazole-4-carboxamide;
N-(5-chloro-6-(2H-1,2,3-triazol-2-yl)pyridin-3-yl)-1-(1-(difluoromethyl)isoquinolin-4-yl)-5-(trifluoromethyl)-1H-pyrazole-4-carboxamide;
N-(5-cyano-6-(2H-1,2,3-triazol-2-yl)pyridin-3-yl)-1-(1-fluoroisoquinolin-4-yl)-5-(trifluoromethyl)-1H-pyrazole-4-carboxamide;
N-(5-chloro-6-(2H-1,2,3-triazol-2-yl)pyridin-3-yl)-1-(1-(methoxymethyl)isoquinolin-4-yl)-5-(trifluoromethyl)-1H-pyrazole-4-carboxamide;
N-(5-chloro-6-(2H-1,2,3-triazol-2-yl)pyridin-3-yl)-1-(2-(1-hydroxyethyl)quinolin-4-yl)-5-(trifluoromethyl)-1H-pyrazole-4-carboxamide;
N-(5-chloro-2-methyl-6-(2H-1,2,3-triazol-2-yl)pyridin-3-yl)-1-(7-methylpyrazolo[1,5-a]pyridin-4-yl)-5-(trifluoromethyl)-1H-pyrazole-4-carboxamide;
1-(1-aminoisoquinolin-4-yl)-N-(5-chloro-2-fluoro-4-(2H-1,2,3-triazol-2-yl)phenyl)-5-(trifluoromethyl)-1H-pyrazole-4-carboxamide;
1-(benzo[d]isothiazol-3-yl)-N-(5-chloro-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(2-fluoroquinolin-5-yl)-5-(trifluoromethyl)-1H-pyrazole-4-carboxamide;
N-(5-chloro-6-(2H-1,2,3-triazol-2-yl)pyridin-3-yl)-1-(isoquinolin-5-yl)-5-(trifluoromethyl)-1H-pyrazole-4-carboxamide;
N-(5-cyano-6-(2H-1,2,3-triazol-2-yl)pyridin-3-yl)-1-(furo[2,3-d]pyrimidin-4-yl)-5-(trifluoromethyl)-1H-pyrazole-4-carboxamide;
N-(5-cyano-6-(2H-1,2,3-triazol-2-yl)pyridin-3-yl)-1-(imidazo[1,2-a]pyridin-3-yl)-5-(trifluoromethyl)-1H-pyrazole-4-carboxamide;
N-(5-chloro-2-methyl-6-(2H-1,2,3-triazol-2-yl)pyridin-3-yl)-1-(imidazo[1,2-a]pyridin-5-yl)-5-(trifluoromethyl)-1H-pyrazole-4-carboxamide;
4-(4-((5-chloro-6-(2H-1,2,3-triazol-2-yl)pyridin-3-yl)carbamoyl)-5-(trifluoromethyl)-1H-pyrazol-1-yl)thieno[2,3-c]pyridine 6-oxide;
1-(benzo[d][1,2,3]thiadiazol-7-yl)-N-(5-chloro-6-(1-methyl-1H-pyrazol-3-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(thiazolo[5,4-d]pyrimidin-7-yl)-5-(trifluoromethyl)-1H-pyrazole-4-carboxamide;
N-(5-cyano-6-(2H-1,2,3-triazol-2-yl)pyridin-3-yl)-1-(2-methylimidazo[1,2-a]pyridin-3-yl)-5-(trifluoromethyl)-1H-pyrazole-4-carboxamide;
N-(5-cyano-6-methoxypyridin-3-yl)-1-(1-oxo-1,2-dihydroisoquinolin-5-yl)-5-(trifluoromethyl)-1H-pyrazole-4-carboxamide;
N-(5-chloro-2-methyl-6-(1-methyl-1H-pyrazol-3-yl)pyridin-3-yl)-1-(1-oxo-1,2-dihydroisoquinolin-5-yl)-5-(trifluoromethyl)-1H-pyrazole-4-carboxamide;
N-(5-cyano-6-(2H-1,2,3-triazol-2-yl)pyridin-3-yl)-1-(quinazolin-4-yl)-5-(trifluoromethyl)-1H-pyrazole-4-carboxamide;
N-(5-chloro-2-methyl-4-(2H-1,2,3-triazol-2-yl)phenyl)-1-(8-fluoroisoquinolin-4-yl)-5-(trifluoromethyl)-1H-pyrazole-4-carboxamide;
N-(5-chloro-2-methyl-4-(2H-1,2,3-triazol-2-yl)phenyl)-1-(1-oxo-1,2-dihydroisoquinolin-5-yl)-5-(trifluoromethyl)-1H-pyrazole-4-carboxamide;
(*R)—N-(5-chloro-6-(2H-1,2,3-triazol-2-yl)pyridin-3-yl)-1-(1-(1-hydroxyethyl)isoquinolin-4-yl)-5-(trifluoromethyl)-1H-pyrazole-4-carboxamide;
N-(5-chloro-6-(2H-1,2,3-triazol-2-yl)pyridin-3-yl)-1-(1-cyanoisoquinolin-4-yl)-5-(trifluoromethyl)-1H-pyrazole-4-carboxamide;
N-(5-chloro-2-fluoro-4-(2H-1,2,3-triazol-2-yl)phenyl)-1-(1-oxo-1,2-dihydroisoquinolin-5-yl)-5-(trifluoromethyl)-1H-pyrazole-4-carboxamide;
N-(5-chloro-6-(2H-1,2,3-triazol-2-yl)pyridin-3-yl)-1-(1-(1-hydroxyethyl)isoquinolin-4-yl)-5-(trifluoromethyl)-1H-pyrazole-4-carboxamide;
N-(5-cyano-2-methyl-4-(2H-1,2,3-triazol-2-yl)phenyl)-1-(1-oxo-1,2-dihydroisoquinolin-5-yl)-5-(trifluoromethyl)-1H-pyrazole-4-carboxamide;
N-(5-chloro-6-(4-methyl-1H-1,2,3-triazol-1-yl)pyridin-3-yl)-1-(quinolin-5-yl)-5-(trifluoromethyl)-1H-pyrazole-4-carboxamide;
N-(5-methyl-6-(2-methyl-2H-tetrazol-5-yl)pyridin-3-yl)-1-(quinolin-5-yl)-5-(trifluoromethyl)-1H-pyrazole-4-carboxamide;
N-(5-chloro-6-(2H-1,2,3-triazol-2-yl)pyridin-3-yl)-1-(7-(difluoromethyl)thieno[2,3-c]pyridin-4-yl)-5-(trifluoromethyl)-1H-pyrazole-4-carboxamide;
N-(5-chloro-6-(2H-1,2,3-triazol-2-yl)pyridin-3-yl)-1-(1-(tetrahydrofuran-2-yl)isoquinolin-4-yl)-5-(trifluoromethyl)-1H-pyrazole-4-carboxamide;
N-(5-chloro-6-(4-(methoxymethyl)-2H-1,2,3-triazol-2-yl)pyridin-3-yl)-1-(quinolin-5-yl)-5-(trifluoromethyl)-1H-pyrazole-4-carboxamide;
N-(4-(4-(aminomethyl)-1H-pyrazol-1-yl)-3-chlorophenyl)-1-(quinolin-5-yl)-5-(trifluoromethyl)-1H-pyrazole-4-carboxamide;
1-(1-aminoisoquinolin-4-yl)-N-(5-cyano-2-methyl-6-(2H-1,2,3-triazol-2-yl)pyridin-3-yl)-5-(trifluoromethyl)-1H-pyrazole-4-carboxamide;
1-(benzo[d]thiazol-4-yl)-N-(5-chloro-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(1-hydroxyisoquinolin-4-yl)-5-(trifluoromethyl)-1H-pyrazole-4-carboxamide;
N-(5-chloro-6-(2H-tetrazol-5-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(8-fluoroquinolin-5-yl)-5-(trifluoromethyl)-1H-pyrazole-4-carboxamide;
N-(5-chloro-6-(2H-1,2,3-triazol-2-yl)pyridin-3-yl)-1-(2-(difluoromethyl)quinolin-4-yl)-5-(trifluoromethyl)-1H-pyrazole-4-carboxamide;
N-(5-cyano-6-(2H-1,2,3-triazol-2-yl)pyridin-3-yl)-1-(8-fluoro-1-(methylamino)isoquinolin-4-yl)-5-(trifluoromethyl)-1H-pyrazole-4-carboxamide;
N-(5-chloro-6-(2H-1,2,3-triazol-4-yl)pyridin-3-yl)-1-(quinolin-5-yl)-5-(trifluoromethyl)-1H-pyrazole-4-carboxamide;
N-(5-chloro-2-fluoro-4-(2H-1,2,3-triazol-2-yl)phenyl)-1-(8-fluoroisoquinolin-4-yl)-5-(trifluoromethyl)-1H-pyrazole-4-carboxamide;
N-(5-chloro-6-(2H-1,2,3-triazol-2-yl)pyridin-3-yl)-1-(1-methyl-2-oxo-1,2-dihydroquinolin-4-yl)-5-(trifluoromethyl)-1H-pyrazole-4-carboxamide;
(*R)—N-(5-chloro-6-(2H-1,2,3-triazol-2-yl)pyridin-3-yl)-1-(1-(3-hydroxypyrrolidin-1-yl)isoquinolin-4-yl)-5-(trifluoromethyl)-1H-pyrazole-4-carboxamide;
N-(5-chloro-6-(2H-1,2,3-triazol-2-yl)pyridin-3-yl)-1-(1-(hydroxymethyl)isoquinolin-4-yl)-5-(trifluoromethyl)-1H-pyrazole-4-carboxamide;
N-(5-cyano-6-(2H-1,2,3-triazol-2-yl)pyridin-3-yl)-1-(3-methylthieno[3,2-b]pyridin-7-yl)-5-(trifluoromethyl)-1H-pyrazole-4-carboxamide;
1-(benzo[d]oxazol-4-yl)-N-(5-cyano-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(4-fluoronaphthalen-1-yl)-5-(trifluoromethyl)-1H-pyrazole-4-carboxamide;
N-(3-chloro-4-(4-(hydroxymethyl)-1H-pyrazol-1-yl)phenyl)-1-(quinolin-5-yl)-5-(trifluoromethyl)-1H-pyrazole-4-carboxamide;
N-(5-cyano-2-methyl-6-(2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
1-(8-aminoquinolin-5-yl)-N-(5-cyano-6-(2H-1,2,3-triazol-2-yl)pyridin-3-yl)-5-(trifluoromethyl)-1H-pyrazole-4-carboxamide;
4-(4-((5-chloro-6-(2H-1,2,3-triazol-2-yl)pyridin-3-yl)carbamoyl)-5-(trifluoromethyl)-1H-pyrazol-1-yl)quinoline 1-oxide;
N-(5-cyano-6-(4-(hydroxymethyl)-2H-1,2,3-triazol-2-yl)pyridin-3-yl)-1-(quinolin-5-yl)-5-(trifluoromethyl)-1H-pyrazole-4-carboxamide;
methyl 2-cyano-4-(1-(quinolin-5-yl)-5-(trifluoromethyl)-1H-pyrazole-4-carboxamido)benzoate;
N-(6-(5-amino-1H-1,2,3-triazol-1-yl)-5-chloropyridin-3-yl)-1-(1-oxo-1,2-dihydroisoquinolin-5-yl)-5-(trifluoromethyl)-1H-pyrazole-4-carboxamide;
N-(5-chloro-6-(4-cyano-2H-1,2,3-triazol-2-yl)pyridin-3-yl)-1-(quinolin-5-yl)-5-(trifluoromethyl)-1H-pyrazole-4-carboxamide;
1-(1-aminoisoquinolin-4-yl)-N-(5-chloro-2-methyl-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(imidazo[1,2-a]pyridin-5-yl)-5-(trifluoromethyl)-1H-pyrazole-4-carboxamide;
1-(benzo[d][1,2,3]thiadiazol-7-yl)-N-(5-cyano-2-methyl-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(8-fluoroimidazo[1,2-a]pyridin-5-yl)-5-(trifluoromethyl)-1H-pyrazole-4-carboxamide;
N-(6-(5-amino-1-methyl-1H-pyrazol-3-yl)-5-cyanopyridin-3-yl)-1-(quinolin-5-yl)-5-(trifluoromethyl)-1H-pyrazole-4-carboxamide;
N-(5-chloro-2-methyl-6-(2H-1,2,3-triazol-2-yl)pyridin-3-yl)-1-(isoquinolin-4-yl)-5-(trifluoromethyl)-1H-pyrazole-4-carboxamide;
N-(5-chloro-6-(4-(hydroxymethyl)-1H-pyrazol-1-yl)pyridin-3-yl)-1-(1-oxo-1,2-dihydroisoquinolin-5-yl)-5-(trifluoromethyl)-1H-pyrazole-4-carboxamide;
N-(5-chloro-6-(2H-1,2,3-triazol-2-yl)pyridin-3-yl)-1-(3-methylimidazo[1,2-a]pyridin-5-yl)-5-(trifluoromethyl)-1H-pyrazole-4-carboxamide;
N-(6-(5-amino-1-methyl-1H-pyrazol-3-yl)-5-chloropyridin-3-yl)-1-(1-oxo-1,2-dihydroisoquinolin-5-yl)-5-(trifluoromethyl)-1H-pyrazole-4-carboxamide;
N-(5-cyano-6-(2H-1,2,3-triazol-2-yl)pyridin-3-yl)-1-(8-fluoroimidazo[1,2-a]pyridin-5-yl)-5-(trifluoromethyl)-1H-pyrazole-4-carboxamide;
N-(5-cyano-6-(2H-1,2,3-triazol-2-yl)pyridin-3-yl)-1-(thieno[3,2-d]pyrimidin-4-yl)-5-(trifluoromethyl)-1H-pyrazole-4-carboxamide;
N-(5-chloro-6-(2H-1,2,3-triazol-2-yl)pyridin-3-yl)-1-(8-fluoro-1-hydroxyisoquinolin-4-yl)-5-(trifluoromethyl)-1H-pyrazole-4-carboxamide;
1-(benzo[d]thiazol-4-yl)-N-(5-cyano-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(quinolin-4-yl)-5-(trifluoromethyl)-1H-pyrazole-4-carboxamide;
N-(5-cyano-6-(2H-1,2,3-triazol-2-yl)pyridin-3-yl)-1-(pyrrolo[1,2-a]pyrazin-1-yl)-5-(trifluoromethyl)-1H-pyrazole-4-carboxamide;
N-(6-(2H-1,2,3-triazol-2-yl)-5-(trifluoromethyl)pyridin-3-yl)-1-(quinolin-5-yl)-5-(trifluoromethyl)-1H-pyrazole-4-carboxamide;
1-(benzo[d][1,2,3]thiadiazol-7-yl)-N-(5-chloro-6-(2H-1,2,3-triazol-2-yl)pyridin-3-yl)-5-(trifluoromethyl)-1H-pyrazole-4-carboxamide;
4-(4-((5-chloro-6-(2H-1,2,3-triazol-2-yl)pyridin-3-yl)carbamoyl)-5-(trifluoromethyl)-1H-pyrazol-1-yl)isoquinoline-1-carboxamide;
N-(5-chloro-6-(2H-1,2,3-triazol-2-yl)pyridin-3-yl)-1-(imidazo[1,2-a]pyridin-5-yl)-5-(trifluoromethyl)-1H-pyrazole-4-carboxamide;
N-(5-chloro-6-(2H-1,2,3-triazol-2-yl)pyridin-3-yl)-1-(pyrrolo[1,2-a]pyrazin-1-yl)-5-(trifluoromethyl)-1H-pyrazole-4-carboxamide;
N-(5-cyano-6-(2-methyl-2H-tetrazol-5-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(1,7-naphthyridin-5-yl)-5-(trifluoromethyl)-1H-pyrazole-4-carboxamide;
2-(3-chloro-5-(1-(quinolin-5-yl)-5-(trifluoromethyl)-1H-pyrazole-4-carboxamido)pyridin-2-yl)-2H-1,2,3-triazole-4-carboxamide;
N-(5-chloro-2-methyl-6-(2H-1,2,3-triazol-2-yl)pyridin-3-yl)-1-(pyrazolo[1,5-a]pyridin-4-yl)-5-(trifluoromethyl)-1H-pyrazole-4-carboxamide;
1-(1-aminoisoquinolin-4-yl)-N-(5-cyano-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(thieno[2,3-c]pyridin-7-yl)-5-(trifluoromethyl)-1H-pyrazole-4-carboxamide;
N-(5-cyano-6-(2H-1,2,3-triazol-2-yl)pyridin-3-yl)-1-(thieno[2,3-d]pyrimidin-4-yl)-5-(trifluoromethyl)-1H-pyrazole-4-carboxamide;
N-(5-chloro-6-(1,3,4-oxadiazol-2-yl)pyridin-3-yl)-1-(quinolin-5-yl)-5-(trifluoromethyl)-1H-pyrazole-4-carboxamide;
N-(5-chloro-6-(2H-1,2,3-triazol-2-yl)pyridin-3-yl)-1-(pyrazolo[1,5-a]pyrazin-4-yl)-5-(trifluoromethyl)-1H-pyrazole-4-carboxamide;
N-(5-chloro-6-(2H-1,2,3-triazol-2-yl)pyridin-3-yl)-1-(7-(1-hydroxyethyl)thieno[2,3-c]pyridin-4-yl)-5-(trifluoromethyl)-1H-pyrazole-4-carboxamide;
N-(5-cyano-6-(2H-1,2,3-triazol-2-yl)pyridin-3-yl)-1-(pyrrolo[2,1-f][1,2,4]triazin-4-yl)-5-(trifluoromethyl)-1H-pyrazole-4-carboxamide;
N-(5-chloro-6-(1-methyl-1H-pyrazol-3-yl)pyridin-3-yl)-1-(1-oxo-1,2-dihydroisoquinolin-5-yl)-5-(trifluoromethyl)-1H-pyrazole-4-carboxamide;
methyl2-(3-chloro-5-(1-(quinolin-5-yl)-5-(trifluoromethyl)-1H-pyrazole-4-carboxamido)pyridin-2-yl)-2H-1,2,3-triazole-4-carboxylate;
N-(5-chloro-6-(4-((dimethylamino)methyl)-2H-1,2,3-triazol-2-yl)pyridin-3-yl)-1-(quinolin-5-yl)-5-(trifluoromethyl)-1H-pyrazole-4-carboxamide;
N-(5-bromo-6-(2H-1,2,3-triazol-2-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(quinolin-4-yl)-5-(trifluoromethyl)-1H-pyrazole-4-carboxamide;
1-(benzo[d][1,2,3]thiadiazol-7-yl)-N-(5-chloro-2-methyl-6-(2H-1,2,3-triazol-2-yl)pyridin-3-yl)-5-(trifluoromethyl)-1H-pyrazole-4-carboxamide;
N-(5-chloro-6-(2H-1,2,3-triazol-2-yl)pyridin-3-yl)-1-(1-cyanoisoquinolin-5-yl)-5-(trifluoromethyl)-1H-pyrazole-4-carboxamide;
N-(5-chloro-6-(4-(hydroxymethyl)-2H-1,2,3-triazol-2-yl)pyridin-3-yl)-1-(quinolin-5-yl)-5-(trifluoromethyl)-1H-pyrazole-4-carboxamide;
N-(5-chloro-6-(4-(hydroxymethyl)-1H-pyrazol-1-yl)pyridin-3-yl)-1-(quinolin-5-yl)-5-(trifluoromethyl)-1H-pyrazole-4-carboxamide;
N-(5-cyano-6-(2H-1,2,3-triazol-2-yl)pyridin-3-yl)-1-(pyrazolo[1,5-a]pyrazin-4-yl)-5-(trifluoromethyl)-1H-pyrazole-4-carboxamide;
N-(5-cyano-2-methyl-6-(2H-1,2,3-triazol-2-yl)pyridin-3-yl)-1-(8-fluoroisoquinolin-4-yl)-5-(trifluoromethyl)-1H-pyrazole-4-carboxamide;
N-(6-(4-amino-2H-1,2,3-triazol-2-yl)-5-chloropyridin-3-yl)-1-(1-oxo-1,2-dihydroisoquinolin-5-yl)-5-(trifluoromethyl)-1H-pyrazole-4-carboxamide;
N-(6-(4-amino-2H-1,2,3-triazol-2-yl)-5-chloropyridin-3-yl)-1-(quinolin-5-yl)-5-(trifluoromethyl)-1H-pyrazole-4-carboxamide;
1-(1-amino-8-fluoroisoquinolin-4-yl)-N-(5-chloro-6-(2H-1,2,3-triazol-2-yl)pyridin-3-yl)-5-(trifluoromethyl)-1H-pyrazole-4-carboxamide;
1-(1-aminoisoquinolin-4-yl)-N-(5-chloro-6-(2H-1,2,3-triazol-2-yl)pyridin-3-yl)-5-(trifluoromethyl)-1H-pyrazole-4-carboxamide;
N-(3-cyano-4-(2H-1,2,3-triazol-2-yl)phenyl)-1-(quinolin-5-yl)-5-(trifluoromethyl)-1H-pyrazole-4-carboxamide;
N-(5-chloro-6-(2H-1,2,3-triazol-2-yl)pyridin-3-yl)-1-(2-oxo-1,2-dihydroquinolin-4-yl)-5-(trifluoromethyl)-1H-pyrazole-4-carboxamide;
1-(4-aminonaphthalen-1-yl)-N-(5-cyano-6-(2H-1,2,3-triazol-2-yl)pyridin-3-yl)-5-(trifluoromethyl)-1H-pyrazole-4-carboxamide;
1-(1-aminoisoquinolin-4-yl)-N-(5-cyano-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(4-fluoro-2-methoxyphenyl)-5-(trifluoromethyl)-1H-pyrazole-4-carboxamide;
N-(5-chloro-6-(2H-1,2,3-triazol-2-yl)pyridin-3-yl)-1-(2-D-quinolin-5-yl)-5-(trifluoromethyl)-1H-pyrazole-4-carboxamide;
N-(5-cyano-6-(2H-1,2,3-triazol-2-yl)pyridin-3-yl)-1-(2-D-quinolin-5-yl)-5-(trifluoromethyl)-1H-pyrazole-4-carboxamide;
N-(3-chloro-4-(2H-1,2,3-triazol-2-yl)phenyl)-1-(1-oxo-1,2-dihydroisoquinolin-5-yl)-5-(trifluoromethyl)-1H-pyrazole-4-carboxamide;
N-(5-cyano-6-(2H-1,2,3-triazol-2-yl)pyridin-3-yl)-1-(thieno[3,2-b]pyridin-7-yl)-5-(trifluoromethyl)-1H-pyrazole-4-carboxamide;
N-(5-bromo-2-methyl-6-(2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
N-(5-chloro-2-methyl-6-(2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
N-(5-chloro-6-(2H-1,2,3-triazol-2-yl)pyridin-3-yl)-1-(pyrazolo[1,5-a]pyridin-4-yl)-5-(trifluoromethyl)-1H-pyrazole-4-carboxamide;
N-(5-chloro-6-(oxazol-2-yl)pyridin-3-yl)-1-(1-oxo-1,2-dihydroisoquinolin-5-yl)-5-(trifluoromethyl)-1H-pyrazole-4-carboxamide;
N-(5-cyano-6-(2H-1,2,3-triazol-2-yl)pyridin-3-yl)-1-(8-fluoroisoquinolin-4-yl)-5-(trifluoromethyl)-1H-pyrazole-4-carboxamide;
N-(5-cyano-6-(2H-1,2,3-triazol-2-yl)pyridin-3-yl)-1-(thieno[2,3-b]pyridin-4-yl)-5-(trifluoromethyl)-1H-pyrazole-4-carboxamide;
N-(5-chloro-6-(2H-1,2,3-triazol-2-yl)pyridin-3-yl)-1-(thieno[2,3-c]pyridin-4-yl)-5-(trifluoromethyl)-1H-pyrazole-4-carboxamide;
N-(5-chloro-6-(4-methyl-2H-1,2,3-triazol-2-yl)pyridin-3-yl)-1-(quinolin-5-yl)-5-(trifluoromethyl)-1H-pyrazole-4-carboxamide;
N-(5-chloro-2-methyl-6-(2H-1,2,3-triazol-2-yl)pyridin-3-yl)-1-(8-fluoroisoquinolin-4-yl)-5-(trifluoromethyl)-1H-pyrazole-4-carboxamide;
1-(benzo[d][1,2,3]thiadiazol-7-yl)-N-(5-cyano-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(5-fluoronaphthalen-1-yl)-5-(trifluoromethyl)-1H-pyrazole-4-carboxamide;
N-(5-chloro-6-(2H-1,2,3-triazol-2-yl)pyridin-3-yl)-1-(8-fluoroisoquinolin-4-yl)-5-(trifluoromethyl)-1H-pyrazole-4-carboxamide;
N-(5-chloro-6-(2H-1,2,3-triazol-2-yl)pyridin-3-yl)-1-(1-hydroxyisoquinolin-5-yl)-5-(trifluoromethyl)-1H-pyrazole-4-carboxamide;
1-(benzo[d]thiazol-7-yl)-N-(5-chloro-6-(2H-1,2,3-triazol-2-yl)pyridin-3-yl)-5-(trifluoromethyl)-1H-pyrazole-4-carboxamide;
N-(5-chloro-6-(4-(hydroxymethyl)-2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
1-(benzo[d]thiazol-7-yl)-N-(5-cyano-6-(2H-1,2,3-triazol-2-yl)pyridin-3-yl)-5-(trifluoromethyl)-1H-pyrazole-4-carboxamide;
N-(5-cyano-6-(2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
1-(1-amino-8-fluoroisoquinolin-4-yl)-N-(5-cyano-6-(2H-1,2,3-triazol-2-yl)pyridin-3-yl)-5-(trifluoromethyl)-1H-pyrazole-4-carboxamide;
2-(3-chloro-5-(1-(quinolin-5-yl)-5-(trifluoromethyl)-1H-pyrazole-4-carboxamido)pyridin-2-yl)-2H-1,2,3-triazole-4-carboxylic acid;
N-(5-cyano-6-(2H-1,2,3-triazol-2-yl)pyridin-3-yl)-1-(furo[3,2-b]pyridin-7-yl)-5-(trifluoromethyl)-1H-pyrazole-4-carboxamide;
4-(4-((5-chloro-6-(2H-1,2,3-triazol-2-yl)pyridin-3-yl)carbamoyl)-5-(trifluoromethyl)-1H-pyrazol-1-yl)thieno[2,3-c]pyridine-7-carboxamide;
1-(7-(3-hydroxyazetidin-1-yl)thieno[2,3-c]pyridin-4-yl)-5-(trifluoromethyl)-N-(2-(trifluoromethyl)pyridin-4-yl)-1H-pyrazole-4-carboxamide;
N-(5-(2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
1-(1-aminoisoquinolin-4-yl)-N-(5-bromo-2-methyl-6-(2H-1,2,3-triazol-2-yl)pyridin-3-yl)-5-(trifluoromethyl)-1H-pyrazole-4-carboxamide;
N-(5-chloro-2-methyl-6-(4-methyl-1H-1,2,3-triazol-1-yl)pyridin-3-yl)-1-(1-oxo-1,2-dihydroisoquinolin-5-yl)-5-(trifluoromethyl)-1H-pyrazole-4-carboxamide;
N-(2-morpholinopyridin-4-yl)-1-(1-oxo-1,2-dihydroisoquinolin-5-yl)-5-(trifluoromethyl)-1H-pyrazole-4-carboxamide;
N-(2-methoxypyridin-4-yl)-1-(1-oxo-1,2-dihydroisoquinolin-5-yl)-5-(trifluoromethyl)-1H-pyrazole-4-carboxamide;
1-(1-aminoisoquinolin-4-yl)-5-(trifluoromethyl)-N-(5-(trifluoromethyl)pyridin-3-yl)-1H-pyrazole-4-carboxamide;
1-(1-aminoisoquinolin-4-yl)-N-(5-chloro-2-methyl-6-(4-methyl-2H-1,2,3-triazol-2-yl)pyridin-3-yl)-5-(trifluoromethyl)-1H-pyrazole-4-carboxamide;
N-(5-ethynyl-2-methyl-6-(2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
N-(5-chloro-2-methyl-6-(4-methyl-2H-1,2,3-triazol-2-yl)pyridin-3-yl)-1-(1-oxo-1,2-dihydroisoquinolin-5-yl)-5-(trifluoromethyl)-1H-pyrazole-4-carboxamide;
N-(5-chloro-6-(2H-1,2,3-triazol-2-yl)pyridin-3-yl)-1-(2-methyl-1-oxo-1,2-dihydroisoquinolin-4-yl)-5-(trifluoromethyl)-1H-pyrazole-4-carboxamide;
(*R)—N-(5-chloro-6-(2H-1,2,3-triazol-2-yl)pyridin-3-yl)-1-(7-(3-hydroxypyrrolidin-1-yl)thieno[2,3-c]pyridin-4-yl)-5-(trifluoromethyl)-1H-pyrazole-4-carboxamide;
N-(5-chloro-2-methyl-6-(2H-1,2,3-triazol-2-yl)pyridin-3-yl)-1-(7-chlorothieno[2,3-c]pyridin-4-yl)-5-(trifluoromethyl)-1H-pyrazole-4-carboxamide;
(*S)—N-(5-chloro-6-(2H-1,2,3-triazol-2-yl)pyridin-3-yl)-1-(7-(3-hydroxypyrrolidin-1-yl)thieno[2,3-c]pyridin-4-yl)-5-(trifluoromethyl)-1H-pyrazole-4-carboxamide;
N-(5-chloro-2-methyl-6-(2H-1,2,3-triazol-2-yl)pyridin-3-yl)-1-(7-cyanothieno[2,3-c]pyridin-4-yl)-5-(trifluoromethyl)-1H-pyrazole-4-carboxamide;
N-(5-chloro-2-methyl-6-(2H-1,2,3-triazol-2-yl)pyridin-3-yl)-1-(7-(3-hydroxyazetidin-1-yl)thieno[2,3-c]pyridin-4-yl)-5-(trifluoromethyl)-1H-pyrazole-4-carboxamide;
4-(4-((5-chloro-2-methyl-6-(2H-1,2,3-triazol-2-yl)pyridin-3-yl)carbamoyl)-5-(trifluoromethyl)-1H-pyrazol-1-yl)thieno[2,3-c]pyridine-7-carboxamide;
N-(5-chloro-6-(2H-1,2,3-triazol-2-yl)pyridin-3-yl)-1-(7-cyclopropylthieno[2,3-c]pyridin-4-yl)-5-(trifluoromethyl)-1H-pyrazole-4-carboxamide;
N-(5-chloro-6-(2H-1,2,3-triazol-2-yl)pyridin-3-yl)-1-(7-methylthieno[2,3-c]pyridin-4-yl)-5-(trifluoromethyl)-1H-pyrazole-4-carboxamide;
N-(5-chloro-6-(2H-1,2,3-triazol-2-yl)pyridin-3-yl)-1-(7-cyanothieno[2,3-c]pyridin-4-yl)-5-(trifluoromethyl)-1H-pyrazole-4-carboxamide;
4-(4-((5-chloro-6-(2H-1,2,3-triazol-2-yl)pyridin-3-yl)carbamoyl)-5-(trifluoromethyl)-1H-pyrazol-1-yl)-N-methylthieno[2,3-c]pyridine-7-carboxamide;
N-(5-chloro-6-(2H-1,2,3-triazol-2-yl)pyridin-3-yl)-1-(7-(3-hydroxyazetidin-1-yl)thieno[2,3-c]pyridin-4-yl)-5-(trifluoromethyl)-1H-pyrazole-4-carboxamide;
N-(5-chloro-6-(2H-1,2,3-triazol-2-yl)pyridin-3-yl)-1-(7-chlorothieno[2,3-c]pyridin-4-yl)-5-(trifluoromethyl)-1H-pyrazole-4-carboxamide;
1-(1-aminoisoquinolin-4-yl)-5-(trifluoromethyl)-N-(2-(trifluoromethyl)pyridin-4-yl)-1H-pyrazole-4-carboxamide;
N-(6-methyl-5-(trifluoromethyl)pyridin-3-yl)-1-(1-oxo-1,2-dihydroisoquinolin-5-yl)-5-(trifluoromethyl)-1H-pyrazole-4-carboxamide;
1-(1-oxo-1,2-dihydroisoquinolin-5-yl)-N-(pyridin-4-yl)-5-(trifluoromethyl)-1H-pyrazole-4-carboxamide;
N-(2-cyclopropylpyridin-4-yl)-1-(1-oxo-1,2-dihydroisoquinolin-5-yl)-5-(trifluoromethyl)-1H-pyrazole-4-carboxamide;
3-chloro-N,N-dimethyl-5-(1-(1-oxo-1,2-dihydroisoquinolin-5-yl)-5-(trifluoromethyl)-1H-pyrazole-4-carboxamido)picolinamide
1-(1-aminoisoquinolin-4-yl)-N-(2-cyanopyridin-4-yl)-5-(trifluoromethyl)-1H-pyrazole-4-carboxamide;
3-chloro-N-methyl-5-(1-(1-oxo-1,2-dihydroisoquinolin-5-yl)-5-(trifluoromethyl)-1H-pyrazole-4-carboxamido)picolinamide;
1-(1-aminoisoquinolin-4-yl)-N-(6-methyl-5-(trifluoromethyl)pyridin-3-yl)-5-(trifluoromethyl)-1H-pyrazole-4-carboxamide;
N-(5-chloropyridin-3-yl)-1-(1-oxo-1,2-dihydroisoquinolin-5-yl)-5-(trifluoromethyl)-1H-pyrazole-4-carboxamide;
1-(thieno[2,3-c]pyridin-4-yl)-5-(trifluoromethyl)-N-(2-(trifluoromethyl)pyridin-4-yl)-1H-pyrazole-4-carboxamide;
1-(8-fluoroimidazo[1,2-a]pyridin-5-yl)-5-(trifluoromethyl)-N-(2-(trifluoromethyl)pyridin-4-yl)-1H-pyrazole-4-carboxamide;
N-(6-cyano-5-(trifluoromethyl)pyridin-3-yl)-1-(1-oxo-1,2-dihydroisoquinolin-5-yl)-5-(trifluoromethyl)-1H-pyrazole-4-carboxamide;
methyl 3-chloro-5-(1-(1-oxo-1,2-dihydroisoquinolin-5-yl)-5-(trifluoromethyl)-1H-pyrazole-4-carboxamido)picolinate;
1-(8-fluoroisoquinolin-4-yl)-5-(trifluoromethyl)-N-(2-(trifluoromethyl)pyridin-4-yl)-1H-pyrazole-4-carboxamide;
N-(2-cyanopyridin-4-yl)-1-(1-oxo-1,2-dihydroisoquinolin-5-yl)-5-(trifluoromethyl)-1H-pyrazole-4-carboxamide;
N-(2-(2-methoxyethoxy)-5-(trifluoromethyl)pyridin-3-yl)-1-(1-oxo-1,2-dihydroisoquinolin-5-yl)-5-(trifluoromethyl)-1H-pyrazole-4-carboxamide;
N-(5-chloro-2-methyl-6-(2H-1,2,3-triazol-2-yl)pyridin-3-yl)-1-(thieno[2,3-c]pyridin-4-yl)-5-(trifluoromethyl)-1H-pyrazole-4-carboxamide;
N-(5-chloro-2-methyl-6-(2H-1,2,3-triazol-2-yl)pyridin-3-yl)-1-(2-oxo-1,2-dihydroquinolin-4-yl)-5-(trifluoromethyl)-1H-pyrazole-4-carboxamide;
N-(5-chloro-2-methyl-6-(2H-1,2,3-triazol-2-yl)pyridin-3-yl)-1-(1-oxo-1,2-dihydroisoquinolin-5-yl)-5-(trifluoromethyl)-1H-pyrazole-4-carboxamide; and
(*S)—N-(5-chloro-6-(2H-1,2,3-triazol-2-yl)pyridin-3-yl)-1-(2-(tetrahydrofuran-2-yl)quinolin-5-yl)-5-(trifluoromethyl)-1H-pyrazole-4-carboxamide;

or an enantiomer, diastereomer, solvate, or a pharmaceutically acceptable salt form thereof.

In particular, the API is a compound of Formula (I), or an enantiomer, diastereomer or pharmaceutically acceptable salt form thereof.

In particular, the API is a compound of Formula (I), or an enantiomer, diastereomer or pharmaceutically acceptable salt form thereof, in amorphous state or dissolved state (i.e. molecular dispersion).

In particular, the API used as starting material in the process to prepare a pharmaceutical formulation as described herein, is a compound of Formula (I), or an enantiomer, diastereomer, solvate, or a pharmaceutically acceptable salt form thereof; while the API in the final pharmaceutical formulation or solid dosage form as defined herein is a compound of Formula (I), or an enantiomer, diastereomer, or pharmaceutically acceptable salt form thereof, in amorphous form or dissolved state.

The compound of Formula (I) may be 1-(1-oxo-1,2-dihydroisoquinolin-5-yl)-5-(trifluoromethyl)-N-(2-(trifluoromethyl)pyridin-4-yl)-1H-pyrazole-4-carboxamide. Compound A corresponds with the following structure:

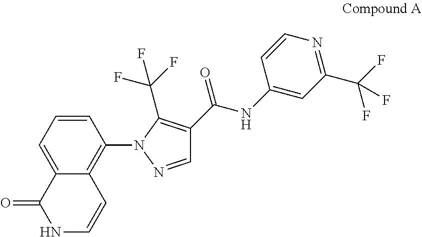 [SMILES: O=C(Nc1ccnc(C(F)(F)F)c1)c1cnn(-c2cccc3c(=O)[nH]ccc23)c1C(F)(F)F]

The API may be Compound A or a solvate or pharmaceutically acceptable salt form thereof. The API may be Compound A or a pharmaceutically acceptable salt form thereof. The API may be Compound A in a solvated form, for example as a monohydrate. In particular, the API is Compound A. In particular, the API is Compound A or a pharmaceutically acceptable salt form thereof in amorphous form or dissolved state. In particular, the API is Compound A in amorphous form or dissolved state.

In particular, the API used as starting material in the process to prepare a pharmaceutical formulation as described herein, is Compound A, a solvated form, or a pharmaceutically acceptable salt form thereof; while the API in the final pharmaceutical formulation or solid dosage form is Compound A or a pharmaceutically acceptable salt form thereof in amorphous form or dissolved state.

In particular, the API used as starting material in the process to prepare a pharmaceutical formulation as described herein, is Compound A in a solvated form, or a pharmaceutically acceptable salt form thereof; while the API in the final pharmaceutical formulation or solid dosage form is Compound A or a pharmaceutically acceptable salt form thereof in amorphous form or dissolved state (i.e. molecular dispersion).

In particular, the API used as starting material in the process to prepare a pharmaceutical formulation as described herein, is Compound A monohydrate or a pharmaceutically acceptable salt form thereof; while the API in the final pharmaceutical formulation or solid dosage form is Compound A or a pharmaceutically acceptable salt form thereof in amorphous form or dissolved state.

In particular, the API used as starting material in the process to prepare a pharmaceutical formulation as described herein, is Compound A monohydrate; while the API in the final pharmaceutical formulation or solid dosage form is Compound A.

In particular, the API used as starting material in the process to prepare a pharmaceutical formulation as described herein, is Compound A monohydrate; while the API in the final pharmaceutical formulation or solid dosage form is Compound A in amorphous form or dissolved state.

Compounds of formula (I) can be synthesised according to the procedures disclosed in WO 2018/119036, which is incorporated herein by reference in its entirety.

It will be appreciated that any of the above discussion relating to active pharmaceutical ingredients may apply to any embodiment of the pharmaceutical formulations, solid dosage forms, processes and treatments described herein. For example, any reference to a MALT1 inhibitor may refer to a compound of formula (I) or an enantiomer, diastereomer, solvate, or pharmaceutically acceptable salt form thereof.

In a particular embodiment, the API in the pharmaceutical formulation as described herein is Compound A, or a pharmaceutically acceptable salt form thereof. In a particular embodiment, the API in the pharmaceutical formulation as described herein is Compound A

In a particular embodiment, the API in the pharmaceutical formulation as described herein is a MALT1 inhibitor in amorphous form or dissolved state. In a particular embodiment, the API in the pharmaceutical formulation as described herein is Compound A or a pharmaceutically acceptable salt form thereof, in amorphous form or dissolved state. In a particular embodiment, the API in the pharmaceutical formulation as described herein is Compound A in amorphous form or dissolved state.

### Solid Dosage Form

The invention also provides a solid dosage form comprising a pharmaceutical formulation as described herein.

The solid dosage form may comprise a capsule encapsulating the pharmaceutical formulation. The capsule may be a hard capsule. The hard capsule may be a gelatin capsule (e.g. ConiSnap®, Licaps®, or Quali-G™) or a hypromellose capsule (e.g. Vegicap®, VCaps®, VCaps® Plus, or Quali-V®). The hard capsule encapsulates a unit dose of the formulation.

The dosage form may be an oral dosage form (e.g. a capsule for oral administration). Alternatively, the dosage form may be an enteral dosage form.

Typically a hard capsule (e.g. a hard gelatin capsule) comprises two part capsule shells, one of which is first filled with the formulation, the other of which is connected to the first in a telescoping manner to close the capsule. The two part capsule shells are typically adhered together by applying solvent (e.g. water or aqueous ethanol) to the interface between the two shells to create a bond between the two part shells.

This differs to the manufacturing processes used for soft gelatin capsules, wherein the formulation is enclosed between half-capsule shells as the soft capsule is formed.

Hard gelatin (hard gel) capsules are generally used for solid, semi-solid, and some compatible liquid formulations, while soft gelatin (soft gel) capsules are generally used for liquid formulations. Hard gel capsules may be preferable for some formulations. Soft gel capsules contain a higher percentage of water than hard gel capsules. This can result in problems when the soft gel contains liquid formulations of poorly water soluble APIs. Water leaching from the soft gel capsule into the formulation may lower the maximum drug loading for that capsule. Higher maximum drug load may be achieved for a poorly water soluble drug when using a hard gel capsule compared to a soft gel capsule.

Additionally, hard gel capsules can more easily be used in blister packs than soft gel capsules, as there is a lower risk of bursting the capsule when forcing it through the foil of the blister.

The solid dosage form may alternatively be a tablet.

The solid dosage form as described herein (e.g. a capsule, e.g. a hard gelatin capsule) may contain about 0.1 mg to about 3000 mg of the API, or any particular amount or range therein, in particular from about 1 mg to about 1000 mg of the API, or any particular amount or range therein, or, more particularly, from about 10 mg to about 500 mg of the API, or any particular amount or range therein, of API in a regimen of about 1 to about (4×) per day for an average (70 kg) human; although, it is apparent to one skilled in the art that the therapeutically effective amount for said API will vary as will the diseases, syndromes, conditions, and disorders being treated.

The solid dosage form as described herein (e.g. a capsule, e.g. a hard gelatin capsule) may contain about 2 to about 1000 mg of the API. In embodiments where the API is 1-(1-oxo-1,2-dihydroisoquinolin-5-yl)-5-(trifluoromethyl)-N-(2-(trifluoromethyl)pyridin-4-yl)-1H-pyrazole-4-carboxamide (Compound A), the solid dosage form may comprise about 2 to about 1000 mg or about 10 to about 200 mg of Compound A. The solid dosage form may comprise 2, 10, 50, 100 or 200 mg of Compound A. The solid dosage form may comprise 2, 10, 50 or 200 mg of Compound A.

The solid dosage form as described herein may comprise 2, 10, 50 or 200 mg of Compound A. In embodiments where the API is 1-(1-oxo-1,2-dihydroisoquinolin-5-yl)-5-(trifluoromethyl)-N-(2-(trifluoromethyl)pyridin-4-yl)-1H-pyrazole-4-carboxamide (Compound A) or a pharmaceutically acceptable salt form thereof, the solid dosage form may comprise about 2 to about 1000 mg or about 10 to about 200 mg of Compound A or a pharmaceutically acceptable salt form thereof. The solid dosage form may comprise 2, 10, 50 or 200 mg of Compound A or a pharmaceutically acceptable salt form thereof. The solid dosage form may comprise 2, 10, 50 or 200 mg of Compound A or a pharmaceutically acceptable salt form thereof.

In a particular embodiment, the solid dosage form is a capsule comprising
a) a polyethylene glycol having a freezing point of at least about 30° C.;
b) an active pharmaceutical ingredient that is soluble in molten polyethylene glycol wherein the polyethylene glycol is as defined in a); and
c) a crystallisation rate inhibitor.

In a particular embodiment, the solid dosage form is a capsule comprising
a) a polyethylene glycol having a freezing point of at least about 30° C.;
b) an active pharmaceutical ingredient that is soluble in molten polyethylene glycol wherein the polyethylene glycol is as defined in a);
c) a crystallisation rate inhibitor; and
d) an antioxidant.

In a particular embodiment, the solid dosage form is a tablet comprising
a) a polyethylene glycol having a freezing point of at least about 30° C.;
b) an active pharmaceutical ingredient that is soluble in molten polyethylene glycol wherein the polyethylene glycol is as defined in a); and
c) a crystallisation rate inhibitor.

In a particular embodiment, the solid dosage form is a tablet comprising
a) a polyethylene glycol having a freezing point of at least about 30° C.;
b) an active pharmaceutical ingredient that is soluble in molten polyethylene glycol wherein the polyethylene glycol is as defined in a);
c) a crystallisation rate inhibitor; and
d) an antioxidant.

In a particular embodiment, the solid dosage form is a capsule consisting of
a) a polyethylene glycol having a freezing point of at least about 30° C.;
b) an active pharmaceutical ingredient that is soluble in molten polyethylene glycol wherein the polyethylene glycol is as defined in a); and
c) a crystallisation rate inhibitor.

In a particular embodiment, the solid dosage form is a capsule consisting of
a) a polyethylene glycol having a freezing point of at least about 30° C.;
b) an active pharmaceutical ingredient that is soluble in molten polyethylene glycol wherein the polyethylene glycol is as defined in a);
c) a crystallisation rate inhibitor; and
d) an antioxidant.

In a particular embodiment, the solid dosage form is a tablet consisting of
a) a polyethylene glycol having a freezing point of at least about 30° C.;
b) an active pharmaceutical ingredient that is soluble in molten polyethylene glycol wherein the polyethylene glycol is as defined in a); and
c) a crystallisation rate inhibitor.

In a particular embodiment, the solid dosage form is a tablet consisting of
a) a polyethylene glycol having a freezing point of at least about 30° C.;
b) an active pharmaceutical ingredient that is soluble in molten polyethylene glycol wherein the polyethylene glycol is as defined in a);
c) a crystallisation rate inhibitor; and
d) an antioxidant.

In a particular embodiment, the solid dosage form is a capsule comprising a pharmaceutical formulation of the present invention.

In a particular embodiment, the solid dosage form is a tablet comprising a pharmaceutical formulation of the present invention.

In an embodiment, the solid dosage form comprises a pharmaceutical formulation, wherein the formulation comprises 2, 10, 50 or 200 mg of 1-(1-oxo-1,2-dihydroisoquinolin-5-yl)-5-(trifluoromethyl)-N-(2-(trifluoromethyl)pyridin-4-yl)-1H-pyrazole-4-carboxamide:

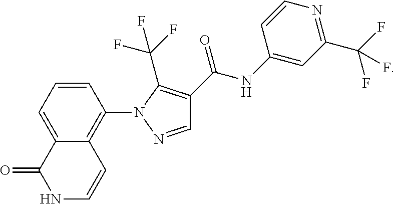 [SMILES: O=C(Nc1ccnc(C(F)(F)F)c1)c1cnn(-c2cccc3c(=O)[nH]ccc23)c1C(F)(F)F]

In an embodiment, the solid dosage form comprises a pharmaceutical formulation, wherein the formulation comprises 2, 10, 50 or 200 mg of 1-(1-oxo-1,2-dihydroisoquinolin-5-yl)-5-(trifluoromethyl)-N-(2-(trifluoromethyl)pyridin-4-yl)-1H-pyrazole-4-carboxamide, or a pharmaceutically acceptable salt form thereof.

The capsule of the solid dosage form may have the role of the crystallisation rate inhibitor. For example, the capsule might be a HPMC capsule.

The crystallisation rate inhibitor might be part of the solid dosage form in tablet form. For example, a HPMC tablet.

The invention also relates to a solid dosage form comprising
a) a polyethylene glycol having a freezing point of at least about 30° C.; and
b) an active pharmaceutical ingredient that is soluble in molten polyethylene glycol wherein the polyethylene glycol is as defined in a);
wherein the solid dosage form is a capsule acting as crystallisation rate inhibitor, e.g. a HPMC capsule.

The invention also relates to a solid dosage form consisting of
a) a polyethylene glycol having a freezing point of at least about 30° C.; and
b) an active pharmaceutical ingredient that is soluble in molten polyethylene glycol wherein the polyethylene glycol is as defined in a);
wherein the solid dosage form is a capsule acting as crystallisation rate inhibitor, e.g. a HPMC capsule.

The invention also relates to a solid dosage form comprising
a) a polyethylene glycol having a freezing point of at least about 30° C.; and
b) an active pharmaceutical ingredient that is soluble in molten polyethylene glycol wherein the polyethylene glycol is as defined in a);
wherein the solid dosage form is in tablet form and wherein the crystallisation rate inhibitor is part of the tablet, e.g. a HPMC tablet.

The invention also relates to a solid dosage form consisting of
a) a polyethylene glycol having a freezing point of at least about 30° C.; and
b) an active pharmaceutical ingredient that is soluble in molten polyethylene glycol wherein the polyethylene glycol is as defined in a);
wherein the solid dosage form is in tablet form and wherein the crystallisation rate inhibitor is part of the tablet, e.g. a HPMC tablet.

For oral administration, a solid dosage form is in particular provided in the form of tablets containing about 1.0, about 10, about 50, about 100, about 150, about 200, about 250, and about 500 milligrams of API; in particular from about 25 mg to about 500 mg of API.

For oral administration, a solid dosage form is in particular provided in the form of capsules containing about 1.0, about 10, about 50, about 100, about 150, about 200, about 250, and about 500 milligrams of API; in particular from about 25 mg to about 500 mg of API.

Advantageously, the API may be administered in a single daily dose, or the total daily dosage may be administered in divided doses of two, three and 4× daily.

Optimal dosages of the pharmaceutical formulation to be administered may be readily determined and will vary with the particular compound used, the mode of administration, the strength of the preparation, and the advancement of the disease, syndrome, condition or disorder. In addition, factors associated with the particular subject being treated, including subject gender, age, weight, diet and time of administration, will result in the need to adjust the dose to achieve an appropriate therapeutic level and desired therapeutic effect. The above dosages are thus exemplary of the average case. There can be, of course, individual instances wherein higher or lower dosage ranges are merited, and such are within the scope of this invention.

The invention also provides a process for preparing a pharmaceutical formulation, as described herein. The process may comprise the steps of
a) forming a melt comprising polyethylene glycol having a freezing point of at least about 30° C., an active pharmaceutical ingredient, and a crystallisation rate inhibitor, wherein the forming a melt step comprises heating polyethylene glycol to a temperature above its freezing point; and
b) cooling the melt below the freezing point of the polyethylene glycol;
to provide a pharmaceutical formulation as described herein.

The invention also provides a process for preparing a solid dosage form, as described herein. The process may comprise the steps of.
a) forming a melt comprising polyethylene glycol having a freezing point of at least about 30° C., an active pharmaceutical ingredient, and a crystallisation rate inhibitor, wherein the forming a melt step comprises heating polyethylene glycol to a temperature above its freezing point;
b) filling a hard capsule (e.g. a gelatin or hypromellose capsule) with the melt; and
c) cooling the filled capsule below the freezing point of the polyethylene glycol;
to provide a solid dosage form as described herein.

In an embodiment, the melt is formed under an inert atmosphere. In another embodiment, the melt is formed under nitrogen.

In an embodiment, the melt further comprises an antioxidant, for example all-rac-alpha-tocopherol. The melt may further comprise one or more pharmaceutically acceptable excipients, as described herein.

The step of forming a melt comprises heating polyethylene glycol to a temperature above its freezing point. The polyethylene glycol may be heated to a temperature of at least about 5, 10, or 15° C. above its freezing point. In particular the polyethylene glycol may be heated to a temperature of at least 5, 10, or 15° C. above the upper limit of its freezing point. The polyethylene glycol may be heated to a temperature of at least about 10° C. above its freezing point. The polyethylene glycol may be heated to a temperature of at most about 20° C. above its freezing point. The polyethylene glycol may be heated to a temperature of at least about 10° C. above the upper limit of its freezing point. The polyethylene glycol may be heated to a temperature of at most about 20° C. above the upper limit of its freezing point. The polyethylene glycol may be heated to a temperature of up to 5 about 70° C., for example about 50° C. to about 70° C. The polyethylene glycol may be heated to a temperature of about 60° C.

The step of forming a melt may comprise adding the API and crystallisation rate inhibitor to molten polyethylene glycol. The step of forming a melt may comprise mixing the polyethylene glycol, API, and crystallisation rate inhibitor and then melting the resulting mixture. In both cases the forming of a melt step comprises heating polyethylene glycol to a temperature above its freezing point.

In particular, the melt is a semi-liquid melt or liquid melt.

In particular, the melt is a liquid melt.

The API used as starting material in step b) of the process to prepare the pharmaceutical formulation according to the present invention, in particular is a crystalline form of Compound A monohydrate, more in particular a crystalline form of Compound A monohydrate producing an X-ray powder diffraction pattern comprising peaks at 16.4, 23.7 and 25.7 degrees two theta±0.2 degrees two theta. The X-ray powder diffraction pattern may further comprise peaks at 13.6, 17.9, 22.6, 24.5, 25.2 and 27.1 degrees two theta±0.2 degrees two theta. The X-ray powder diffraction pattern may further comprise at least one peak selected from 8.3, 8.6, 11.5, 14.0, 15.4, 17.5, 19.7, 22.0, 22.2, 24.0 and 29.9 degrees two theta±0.2 degrees two theta. The X-ray powder diffraction pattern may comprise peaks at 8.3, 8.6, 11.5, 13.6, 14.0, 15.4, 16.4, 17.5, 17.9, 19.7, 22.6, 23.7, 24.5, 25.2, 25.7, and 27.1 degrees two theta±0.2 degrees two theta. The X-ray powder diffraction pattern may comprise peaks at 11.5, 16.4, 19.7, 23.7 and 25.7 degrees two theta±0.2 degrees two theta.

The hard capsule may be filled using a capsule filling machine hopper. The machine hopper may be preheated to a temperature above the freezing point of the polyethylene glycol, wherein the temperature is as described above.

The filled capsule is cooled to a temperature below the freezing point of the polyethylene glycol so that the pharmaceutical formulation solidifies. The capsule may be stored at room temperature (e.g. 25° C.) following the filling step, to ensure the formulation solidifies.

The process may further comprise the step of packaging the capsules in bottles (e.g. HDPE bottles), followed by induction sealing. Alternatively, the process may further comprise the step of sealing the capsules in blister packs.

This process may be advantageous compared to traditional processes for manufacturing solid dosage forms. The molten formulation can be easily dispensed into a capsule and then allowed to solidify. This reduces the number of steps usually associated with the manufacture of solid formulations.

A solid dosage form of the invention may be prepared using a spray congealing process, comprising the steps of: a) forming a melt comprising polyethylene glycol having a freezing point of at least about 30° C., an active pharmaceutical ingredient, and a crystallisation rate inhibitor; and b) atomizing the melt into cold nitrogen. The atomised melt may be compressed into tablets.

A solid dosage form of the invention may be prepared by a screw granulation process, for example using twin-screw extruders that continuously mix and granulate the polyethylene glycol having a freezing point of at least about 30° C., active pharmaceutical ingredient, and crystallisation rate inhibitor (and optionally maltodextrin). The resulting granules may be compressed into tablets.

A solid dosage form of the invention may be prepared by loading a melt of polyethylene glycol having a freezing point of at least about 30° C., active pharmaceutical ingredient, and crystallisation rate inhibitor onto a porous clay-type particle, such as magnesium aluminometasilicate (e.g. Neusilin®) or silica, to obtain a powder which may be compressed into tablets.

It will be appreciated that any of the above discussion relating to solid dosage forms and processes for their preparation may apply to any embodiments of solid dosage forms, processes and treatments described herein.

### Methods of Treatment

The pharmaceutical formulations described herein may be administered in any of the foregoing dosage forms and regimens or by means of those dosage forms and regimens established in the art whenever use of the pharmaceutical formulation is required for a subject in need thereof.

The pharmaceutical formulations and dosage forms of the present invention are useful in methods for treating, ameliorating and/or preventing a disease, a syndrome, a condition or a disorder in a subject in need thereof. Such methods comprise, consist of and/or consist essentially of administering to a subject, including an animal, a mammal, and a human in need of such treatment, amelioration and/or prevention, a therapeutically effective amount of a formulation or dosage form described herein. In embodiments in which the active pharmaceutical ingredient is a MALT1 inhibitor, the pharmaceutical formulations and dosage forms of the present invention are useful in methods for treating, ameliorating and/or preventing a disease, a syndrome, a condition that is affected by the inhibition of MALT1.

One embodiment of the present invention is directed to a method of treating a MALT1-dependent or MALT1-mediated disease or condition in a subject in need thereof, including an animal, a mammal, and a human in need of such treatment, comprising administering to the subject a therapeutically effective amount of a pharmaceutical formulation or dosage form described herein.

In another embodiment, the MALT1-dependent or MALT1-mediated disease or condition is selected from cancers of hematopoietic origin or solid tumors such as chronic myelogenous leukemia, myeloid leukemia, non-Hodgkin lymphoma, and other B cell lymphomas.

In particular, pharmaceutical formulations and dosage forms of the invention are useful for treating or ameliorating diseases, syndromes, conditions, or disorders such as diffuse large B-cell lymphoma (DLBCL), mantle cell lymphoma (MCL), follicular lymphoma (FL), and mucosa-associated lymphoid tissue (MALT) lymphoma.

More particularly, pharmaceutical formulations and dosage forms of the invention are useful for treating or ameliorating diffuse large B-cell lymphoma (DLBCL), mantle cell lymphoma (MCL), follicular lymphoma (FL), and mucosa-associated lymphoid tissue (MALT) lymphoma, comprising administering to a subject in need thereof a therapeutically effective amount of a pharmaceutical formulation or dosage form described herein.

Further, pharmaceutical formulations and dosage forms described herein are useful for treating or ameliorating an immunological disease, syndrome, disorder, or condition selected from the group consisting of rheumatoid arthritis (RA), psoriatic arthritis (PsA), psoriasis (Pso), ulcerative colitis (UC), Crohn's disease, systemic lupus erythematosus (SLE), asthma, and chronic obstructive pulmonary disease (COPD).

In an embodiment, cancers that may benefit from a treatment with pharmaceutical formulations and dosage forms described herein include, but are not limited to, lymphomas, leukemias, carcinomas, and sarcomas, e.g. non-Hodgkin's lymphoma (NHL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head & neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, and GIST (gastrointestinal stromal tumor).

In another embodiment, pharmaceutical formulations and dosage forms of the invention may be used for the treatment of immunological diseases including, but not limited to, autoimmune and inflammatory disorders, e.g. arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, and polymyositis.

One embodiment of the present invention is directed to a method of treating a disease, syndrome, condition, or disorder, wherein said disease, syndrome, condition, or disorder is affected by the inhibition of MALT1, comprising administering to a subject in need thereof a therapeutically effective amount of a pharmaceutical formulation or dosage form described herein.

In a further embodiment, the disease, syndrome, condition, or disorder is selected from the group consisting of diffuse large B-cell lymphoma (DLBCL), mantle cell lymphoma (MCL), follicular lymphoma (FL), and mucosa-associated lymphoid tissue (MALT) lymphoma, rheumatoid arthritis (RA), psoriatic arthritis (PsA), psoriasis (Pso), ulcerative colitis (UC), Crohn's disease, systemic lupus erythematosus (SLE), asthma, and chronic obstructive pulmonary disease (COPD).

In a further embodiment, the disease, syndrome, condition, or disorder is selected from the group consisting of diffuse large B-cell lymphoma (DLBCL), mantle cell lymphoma (MCL), follicular lymphoma (FL), mucosa-associated lymphoid tissue (MALT) lymphoma, marginal zone lymphoma, chronic lymphocytic leukemia (CLL), small lymphocytic lymphoma (SLL), and Waldenström macroglobulinemia.

In one embodiment, the present invention is directed to a method of treating a disease, syndrome, condition, or disorder selected from the group consisting of diffuse large B-cell lymphoma (DLBCL), mantle cell lymphoma (MCL), follicular lymphoma (FL), and mucosa-associated lymphoid tissue (MALT) lymphoma, rheumatoid arthritis (RA), psoriatic arthritis (PsA), psoriasis (Pso), ulcerative colitis (UC), Crohn's disease, systemic lupus erythematosus (SLE), asthma, and chronic obstructive pulmonary disease (COPD), comprising administering to a subject in need thereof a therapeutically effective amount of a pharmaceutical formulation or dosage form described herein.

In another embodiment, the present invention is directed to a method of treating a disease, syndrome, condition, or disorder selected from the group consisting of diffuse large B-cell lymphoma (DLBCL), mantle cell lymphoma (MCL), follicular lymphoma (FL), mucosa-associated lymphoid tissue (MALT) lymphoma, marginal zone lymphoma, chronic lymphocytic leukemia (CLL), small lymphocytic lymphoma (SLL), and Waldenström macroglobulinemia, comprising administering to a subject in need thereof a therapeutically effective amount of a pharmaceutical formulation or dosage form described herein. In a further embodiment, the disease, syndrome, condition, or disorder is non-Hodgkin's lymphoma (NHL). In a further embodiment, the non-Hodgkin's lymphoma (NHL) is B-cell NHL.

In another embodiment, the present invention is directed to a pharmaceutical formulation described herein for the preparation of a medicament for treating a disease, syndrome, disorder or condition selected from the group consisting of diffuse large B-cell lymphoma (DLBCL), mantle cell lymphoma (MCL), follicular lymphoma (FL), and mucosa-associated lymphoid tissue (MALT) lymphoma, rheumatoid arthritis (RA), psoriatic arthritis (PsA), psoriasis (Pso), ulcerative colitis (UC), Crohn's disease, systemic lupus erythematosus (SLE), asthma, and chronic obstructive pulmonary disease (COPD), in a subject in need thereof.

In another embodiment, the present invention is directed to a pharmaceutical formulation described herein for the preparation of a medicament for treating a disease, syndrome, condition, or disorder selected from the group consisting of diffuse large B-cell lymphoma (DLBCL), mantle cell lymphoma (MCL), follicular lymphoma (FL), mucosa-associated lymphoid tissue (MALT) lymphoma, marginal zone lymphoma, chronic lymphocytic leukemia (CLL), small lymphocytic lymphoma (SLL), and Waldenström macroglobulinemia, in a subject in need thereof. In a further embodiment, the disease, syndrome, condition, or disorder is non-Hodgkin's lymphoma (NHL). In a further embodiment, the non-Hodgkin's lymphoma (NHL) is B-cell NHL.

In another embodiment, a pharmaceutical formulation or dosage form described herein is for use in a method for treating a disorder selected from the group consisting of diffuse large B-cell lymphoma (DLBCL), mantle cell lymphoma (MCL), follicular lymphoma (FL), and mucosa-associated lymphoid tissue (MALT) lymphoma, rheumatoid arthritis (RA), psoriatic arthritis (PsA), psoriasis (Pso), ulcerative colitis (UC), Crohn's disease, systemic lupus erythematosus (SLE), asthma, and chronic obstructive pulmonary disease (COPD), in a subject in need thereof.

In another embodiment, a pharmaceutical formulation or dosage form described herein is for use in a method for treating a disease, syndrome, condition, or disorder selected from the group consisting of diffuse large B-cell lymphoma (DLBCL), mantle cell lymphoma (MCL), follicular lymphoma (FL), mucosa-associated lymphoid tissue (MALT) lymphoma, marginal zone lymphoma, chronic lymphocytic leukemia (CLL), small lymphocytic lymphoma (SLL), and Waldenström macroglobulinemia, in a subject in need thereof. In a further embodiment, the disease, syndrome, condition, or disorder is non-Hodgkin's lymphoma (NHL), in a subject in need thereof. In a further embodiment, the non-Hodgkin's lymphoma (NHL) is B-cell NHL.

In another embodiment of the present invention, the pharmaceutical formulations described herein may be employed in combination with one or more other medicinal agents, more particularly with other anti-cancer agents, e.g. chemotherapeutic, anti-proliferative or immunomodulating agents, or with adjuvants in cancer therapy, e.g. immunosuppressive or anti-inflammatory agents.

It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All possible combinations of the above-indicated embodiments are considered to be embraced within the scope of this invention.

Reference is now made to the following examples, which illustrate the invention in a non-limiting fashion.

### GENERAL SYNTHETIC METHODS

Representative compounds of the present invention can be synthesized in accordance with the general synthetic methods described below and illustrated in the schemes and examples that follow. Since the schemes are an illustration, the invention should not be construed as being limited by the chemical reactions and conditions described in the schemes and examples. Compounds analogous to the target compounds of these examples can be made according to similar routes. The disclosed compounds are useful as pharmaceutical agents as described herein. The various starting materials used in the schemes and examples are commercially available or may be prepared by methods well within the skill of persons versed in the art.

Abbreviations used in the instant specification, particularly the schemes and examples, are as follows:
Ac₂O acetic anhydride
AcOH acetic acid
API active pharmaceutical ingredient
BOP (benzotriazol-1-yloxy)tris(dimethylamino)phosphonium hexafluorophosphate
BPO benzoyl peroxide
Bu butyl
cat. catalyst
DBU 1,8-diazabicyclo[5.4.0]undec-7-ene
DCM dichloromethane
DMA dimethylacetamide
DIPEA N,N-diisoproylethylamine
DMF dimethylformamide
DMSO dimethyl sulfoxide
DSC differential scanning calorimetry
Et ethyl
EtOH ethyl alcohol
FaSSIF fasted-state simulated intestinal fluid
h hour(s)
HATU O-(7-azabenzotriazol-1-yl)-N,N,N′,N′-tetramethyluronium hexafluorophosphate
HDPE high-density polyethylene
HPLC high performance liquid chromatography
LDPE low-density polyethylene
LED light-emitting diode
m-CPBA meta-chloroperoxybenzoic acid
Me methyl
MeOH methyl alcohol
mg milligram
min minute
NH₄Cl ammonium chloride
NMP N-methyl-2-pyrrolidone
Pd(dppf)Cl₂ [1,1′-bis(diphenylphosphino)ferrocene]dichloropalladium
Pd(PPh₃)₄ tetrakis(triphenylphosphine)palladium
PK pharmacokinetic
PPh₃ triphenyl phosphine
Pt/C platinum on charcoal
PVPVA polyvinylpyrrolidone-vinyl acetate copolymer
TMPMgCl.LiCl 2,2,6,6-tetramethylpiperidinylmagnesium chloride lithium chloride complex
TsOH toluenesulfonic acid
rpm revolutions per minute
rt or RT room temperature
TBAF tetrabutyl ammonium fluoride
TEA triethylamine
TMSI iodotrimethylsilane
t-Bu tert-butyl
TFA trifluoroacetic acid
TFAA trifluoroacetic anhydride
THF tetrahydrofuran
Xantphos 4,5-Bis(diphenylphosphino)-9,9-dimethylxanthene
XRPD X-ray powder diffraction

Compounds of Formula (Ia) wherein R₇ is hydrogen, may be prepared according to the process outlined in Scheme 1.

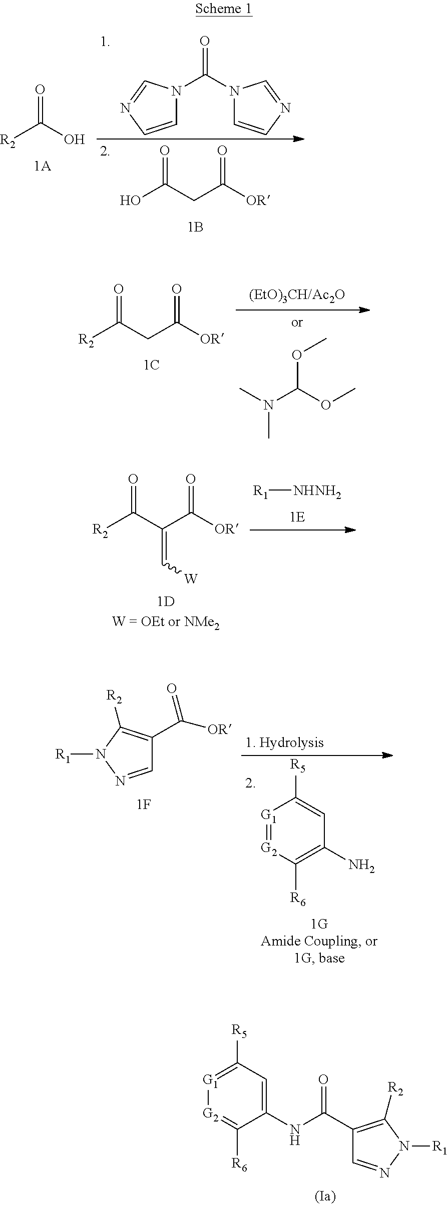

A carboxylic acid of formula (1A) may be treated with carbonyldiimidazole followed by addition of a mono-ester of malonic acid of formula (1B), wherein R′ is C₁₋₄alkyl, and a base, such as isopropylmagesium chloride, to yield a ketoester of formula (1C). Condensation with triethyl orthoformate in acetic anhydride or with 1,1-dimethoxy-N,N-dimethylmethanamine may yield a 2-ethoxymethylidene-3-oxo ester (or 2-(dimethylamino)methylidene-3-oxo ester) of formula (1D). A compound of formula (1D) may be reacted with a hydrazine of formula (1E) to provide a pyrazole of formula (1F). Hydrolysis of the ester group may be effected via by treatment with aqueous sodium hydroxide in the presence of an alcohol co-solvent, to provide the corresponding carboxylic acid intermediate, which, subsequently, may be converted to a compound of Formula (I) upon amide coupling with a compound of formula (1G). The amide coupling may be carried out, for example, in the presence of phosphorus oxychloride in pyridine to afford the corresponding acid chloride, followed by treatment with a compound of formula (1G), in the presence of a base. In one embodiment, the amide coupling reaction is carried out in the presence of a suitable amide coupling reagent such as HATU, in the presence of a base such as, but not limited to, diisopropylethyl amine.

Alternatively, the pyrazole ester of formula (1F) may be directly converted to a compound of Formula (I) via treatment with a compound of formula (1G) and a base, such as potassium tert-butoxide.

An alternate route to compounds of Formula (Ia) wherein R₇ is hydrogen, is illustrated in Scheme 2.

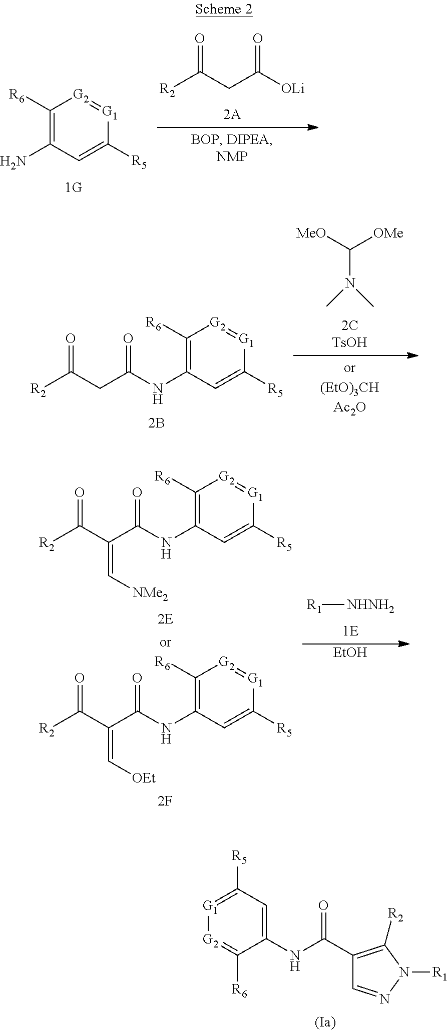

Aniline (1G) may be coupled with a lithium acetoacetate of formula (2A) in the presence of coupling reagent such as BOP, a base such as DIPEA, and a solvent such as NMP, to provide a compound of formula (2B). A compound of formula (2B) may then be reacted with DMF-DMA (2C) in the presence of an acid, such as TsOH, or reacted with triethoxymethane (2D) in AcOH to afford a compound of formula (2E) or (2F), respectively. A compound of formula (2E) or (2F) may then be treated with a hydrazine of formula (1E) to afford a compound of Formula (I).

Scheme 3 illustrates the preparation of certain hydrazine intermediates of formula (1E), useful for the preparation of compounds of Formula (I) of the present invention.

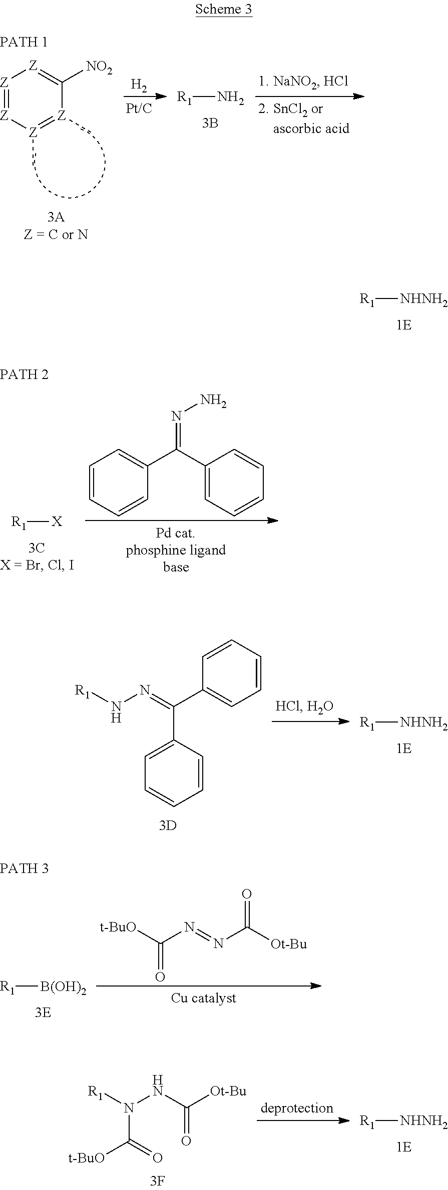

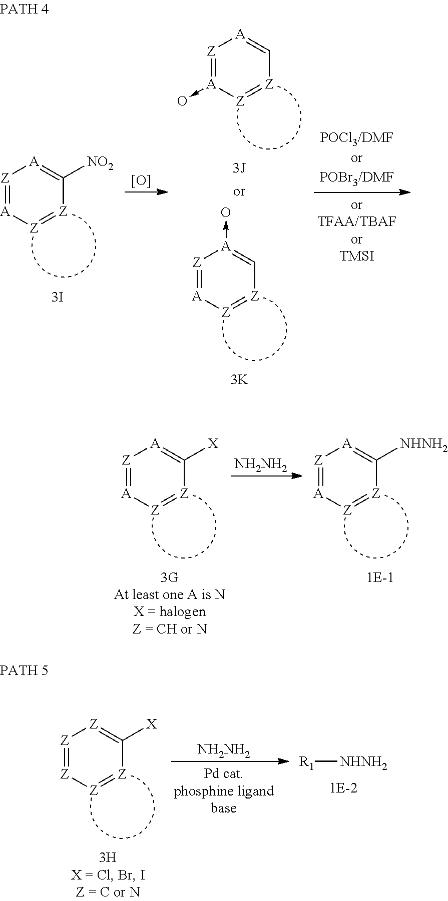

A heteroaryl amine of formula (3B) may be converted to a heteroaryl diazonium salt via treatment with sodium nitrite under acidic conditions. This intermediate may be reduced, using a reductant such as tin (II) chloride or ascorbic acid, to form the hydrazine of formula (1E). For heteroaryl amines of formula (3B) that are not commercially available, they may be accessed by reduction of the heteronitroarene (3A) using hydrogen and Pt/C or other conventional nitro-reducing conditions (path one).

R₁-substituted chlorides, bromides, and iodides may undergo a palladium catalyzed Buchwald Hartwig coupling with benzophenone hydrazine, in the presence of a ligand, such as Xantphos, and a base, such as sodium tert-butoxide, to form a hydrazine of formula (3D). Acidic hydrolysis may afford the hydrazine of formula (1E) (path two).

R₁-substituted boronic acids may also serve as a precursor to compounds of formula (1E) by the route shown in path three. A boronic acid of formula (3E) may undergo a Cu²⁺-catalyzed (such as Cu(OAc)₂, TEA in CH₂Cl₂) addition to di-tert-butylazodicarboxylate to afford an intermediate of formula (3F), which may be deprotected under acidic conditions to yield the compound of formula (1E). Heteroaryl hydrazines of formula (1E-1), having a nitrogen atom in the ortho- or para- position with respect to the hydrazine functionality, may be prepared via direct displacement of a halogen with hydrazine or hydrazine hydrate. (Hetero) haloarenes of formula (3G) that are not commercially available may be prepared from their corresponding (hetero)arenes (3I), with an oxidant such as mCPBA, to form the N-oxide (3J) (or (3K)) that may then be converted to (hetero) haloarene 3G via treatment with POCl₃ and DMF, POBr₃/DMF, TFAA/TBAF, or TMSI (path four). Alternatively, halogenated (hetero)arenes of formula (3H) may undergo palladium-catalyzed cross-coupling with hydrazine to directly furnish intermediate (1E-2) (path five).

Scheme 4 illustrates multiple pathways available for the synthesis of intermediate (1G-1), wherein G₁ is C(R₄).

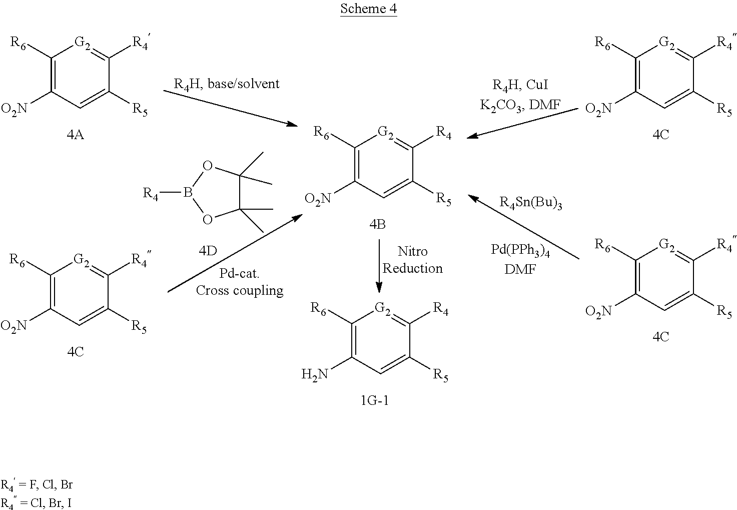

Compound (B-1) may be reacted with a compound of formula R₄H in the presence of a base, such as Cs₂CO₃, in a solvent, such as DMF, to yield a compound of formula (4B). Alternatively, a compound of formula (4C) may be treated with a crossing coupling reagent, such as a boron reagent of formula (4D) or a tin reagent of formula R₄Sn(Bu)₃; in the presence of a palladium catalyst, including but not limited to, Pd(dppf)Cl₂ or Pd(PPh₃)₄; in a suitable solvent or solvent system such as DMF, dioxane/water, or the like; to produce a compound of formula (4B). Another suitable pathway includes the reaction of a compound of formula (4C) with a compound of formula R₄H, in the presence of a coupling reagent such as CuI, with a base such as Cs₂CO₃, and in a solvent such as DMF, to afford a compound of formula (4B). A compound of formula (4B) may be reduced to a compound of formula (1G-1) using a reducing agent such as Zn or Fe in the presence of NH₄Cl, in a solvent such as MeOH.

Scheme 5 illustrates the preparation of certain compounds of Formula (I) wherein R₆ is other than hydrogen.

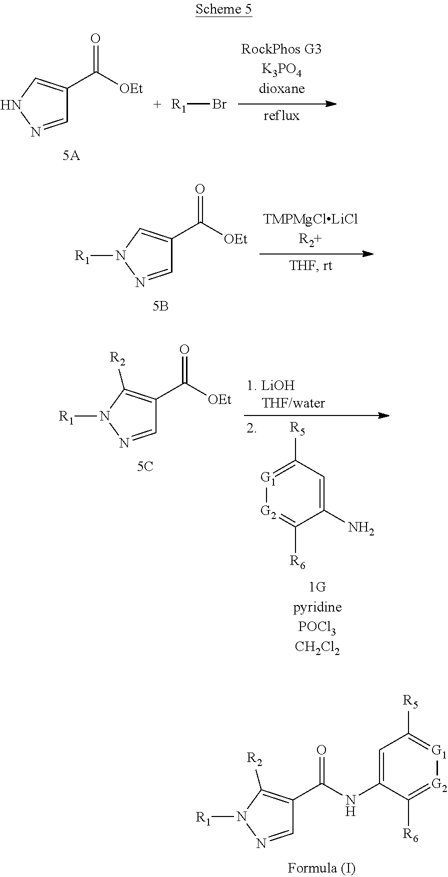

Scheme 6 illustrates the preparation of certain compounds of Formula (I) of the present invention.

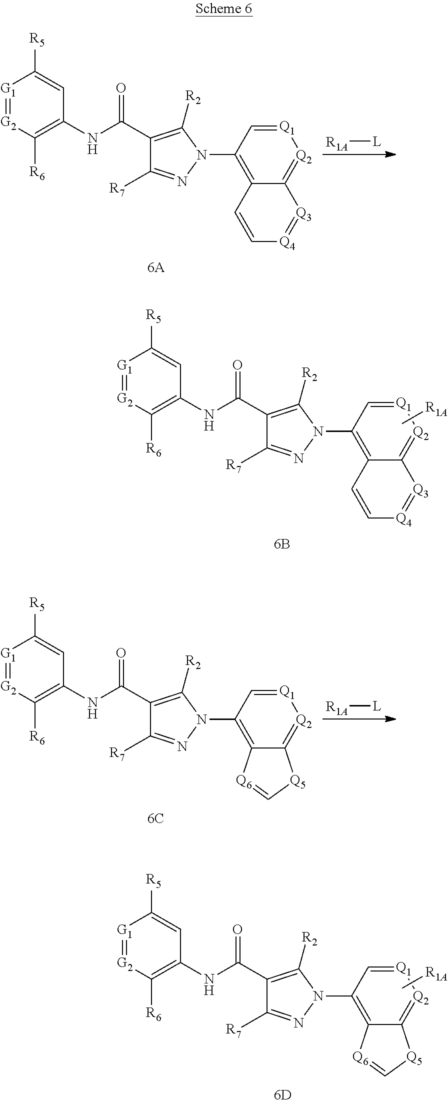

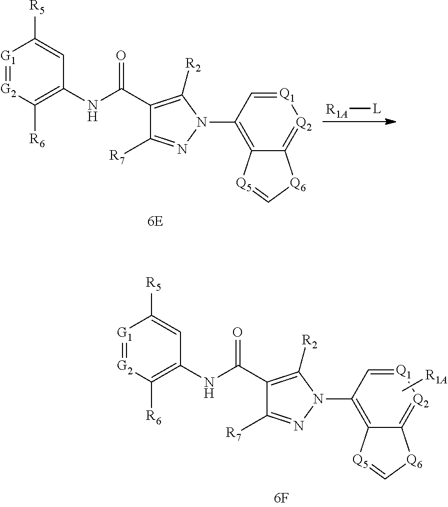

In the instance when L is H, alkylation of compounds of formulae 6A, 6C and 6E may occur via formation of a radical from R_{1A}-L, generated by treatment with ammonium persulfate and (IR[DF(CF₃)PPY]₂(DTBPY))PF₆, in a mixture of water and CH₃CN or DMSO and TFA, under irradiation with blue LED.

Alternatively, in the instance when L is H, alkylation of compounds of formulae 6A, 6C and 6E may occur via formation of a radical from R_{1A}-L, generated by treatment with BPO and (IR[DF(CF₃)PPY]₂(DTBPY))PF₆ in MEOH and TFA, under irradiation with blue LED.

When L is H, alkylation of compounds of formulae 6A, 6C and 6E may occur via formation of a radical from R_{1A}-L, generated by treatment with iron(II)sulfate heptahydrate and hydrogen peroxide, in a mixture of water and CH₃CN or DMSO and H₂SO₄.

When L is a zinc sulfonate, alkylation of compounds of formulae 6A, 6C and 6E may occur via formation of a radical from R_{1A}-L, generated by treatment with tert-butyl hydroperoxide, in a mixture of water and DCM and TFA.

Likewise, when L is —COOH or a BF₃-salt, alkylation of compounds of formulae 6A, 6C and 6E may occur via formation of a radical from R_{1A}-L, generated by treatment with ammonium persulfate and silver nitrate, in a mixture of water and DCM or CH₃CN or DMSO or dioxane and TFA.

Compounds of formulae 6A, 6C and 6E may also be converted to their corresponding N-oxides via treatment with an oxidizing agent such as m-CPBA in DCM or THF. Said N-oxides by optionally be converted to their corresponding ortho —CN derivatives using trimethylsilyl cyanide and DBU, in a solvent such as THF. Said N-oxides may also be converted to their alkoxy or cycloalkoxy derivatives by the action of tosylanhydride, Na₂CO₃ and an appropriately substituted alkyl-OH or cycloalkyl-OH reagent.

Alternatively, the N-oxides of compounds of formulae 6A, 6C and 6E may be converted to their corresponding ortho-chloro derivatives by the action of POCl₃, optionally in a solvent such as CHCl₃, which may be used as an intermediate for the preparation of C₁₋₆alkylthio, C₁₋₆cycloalkylthio, and sulfur-linked heterocyclic rings of the present invention. Similarly, the ortho-chloro derivatives may be reacted with appropriately substituted amines to afford C₁₋₆alkylamino, C₁₋₆cycloalkylamino, or N-linked heterocyclic rings of the present invention. Or, the ortho-chloro derivatives may undergo a Suzuki-type reaction in a subsequent step, with an appropriately substituted corresponding alkyl- or cycloalkyl-boronic acid to form a compound of Formula (I).

Compounds of Formula (I) can be synthesised according to the procedures disclosed in WO 2018/119036, which is incorporated herein by reference in its entirety.

### EXAMPLES

### Example 1: Preparation of crystalline 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A) monohydrate

1-(1-oxo-1,2-dihydroisoquinolin-5-yl)-5-(trifluoromethyl)-N-[2-(trifluoromethyl)pyridin-4-yl]-1H-pyrazole-4-carboxamide (100 g) obtained by a procedure analogous to the synthesis method as described in Example 158 of WO 2018/119036 was charged in a flask (R1) together with ethanol (150-170 mL) and ethyl acetate (80-100 mL). The obtained mixture was heated to 400-50° C. and stirred for 0.5-2 hours. Water (4-7 mL) was then added and the water content was measured by Karl Fischer titration. The content of R1 was warmed to 40°-55° C. and filtered into a second flask (R2) pre-heated at 40°-55° C. R1 was rinsed with ethyl acetate (80-100 mL) at 40°-50° C. and the content filtered into R2. n-Heptane (340-410 mL) was charged into R2 in about 20-40 min. maintaining 40°−55° C. The obtained solution was seeded with 1.9-2.1 g of crystalline monohydrate of Compound A and the obtained mixture was stirred at 40°−55° C. for 4-8 hours. n-heptane (680-750 mL) was added in 10-15 hours maintaining 40°-55° C.; the obtained mixture was stirred for additional 2-5 hours at 40°-55° C., then it was cooled down to 20°-25° C. for 7-13 hours. The suspension was stirred at 20°-25° C. for 12-18 h, then it was filtered and washed with n-Heptane (180-250 mL). After drying under vacuum at 45°-55° C. for 15-22 hours, crystalline 1-(1-oxo-1,2-dihydroisoquinolin-5-yl)-5-(trifluoromethyl)-N-[2-(trifluoromethyl)pyridin-4-yl]-1H-pyrazole-4-carboxamide monohydrate was obtained with an 80% yield.

### Example 1b: Preparation of crystalline 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A) monohydrate

1-(1-oxo-1,2-dihydroisoquinolin-5-yl)-5-(trifluoromethyl)-N-[2-(trifluoromethyl)pyridin-4-yl]-1H-pyrazole-4-carboxamide monohydrate (25 g) obtained by a procedure analogous to the synthesis method as described in Example 158 of WO 2018/119036 was charged in a flask (R1) together with water (2.5-4.5 mL) and isopropyl alcohol (IPA) (100 mL). The obtained mixture was heated to 50° C. and stirred for 0.5-2 hours. n-Heptane (125 mL) was charged into R1. The obtained solution was seeded with 500 mg of crystalline monohydrate of Compound A and the obtained mixture was stirred at 50° C. for 72 hours. n-Heptane (275 mL) was added in 12 hours maintaining 50° C.; the obtained mixture was stirred for additional 58 hours at 50° C., then it was cooled down to 200-25° C. for 2 hours. The suspension was stirred at 200-25° C. for 94 h, then it was filtered and washed with n-heptane (100 mL). After drying under vacuum at 50° C. for 24 hours, crystalline 1-(1-oxo-1,2-dihydroisoquinolin-5-yl)-5-(trifluoromethyl)-N-[2-(trifluoromethyl)pyridin-4-yl]-1H-pyrazole-4-carboxamide monohydrate was obtained with an 90% yield.

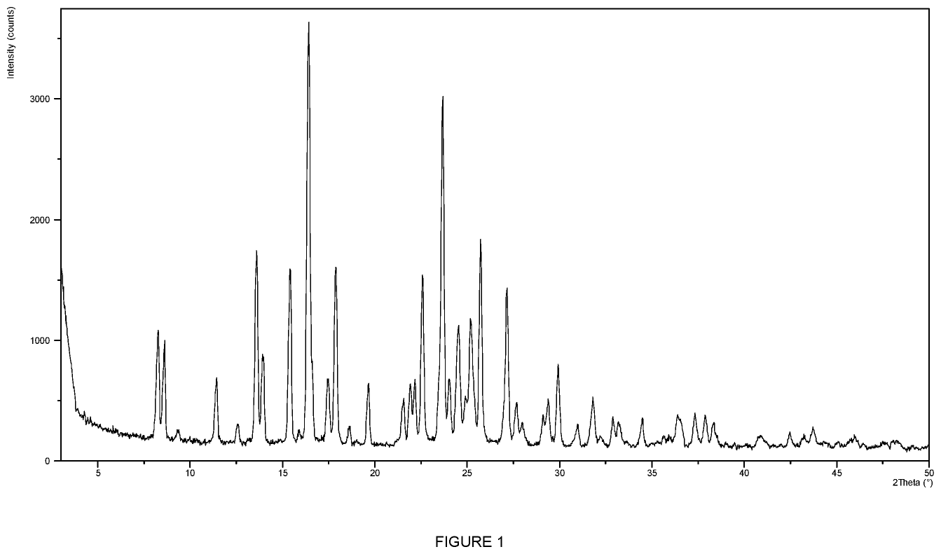

The crystalline monohydrate was characterized by XRPD (see FIG. 1). Table 1 provides peak listings and relative intensities for the XPRD.

TABLE 1

| Pos. [°2Th.] | Rel. Int. [%] |
|---|---|
| 8.2904 | 25.26 |
| 8.6250 | 23.96 |
| 9.3485 | 2.24 |
| 11.4511 | 14.20 |
| 12.5682 | 4.31 |
| 13.6202 | 45.95 |
| 13.9754 | 21.49 |
| 15.4397 | 41.22 |
| 15.8867 | 3.10 |
| 16.4426 | 100.00 |
| 16.6283 | 17.71 |
| 17.5110 | 14.58 |
| 17.9121 | 41.41 |
| 18.6250 | 4.18 |
| 19.6673 | 14.48 |
| 21.5675 | 11.28 |
| 21.9258 | 14.96 |
| 22.1775 | 15.69 |
| 22.5940 | 41.75 |
| 23.6809 | 85.80 |
| 24.0437 | 15.69 |
| 24.5412 | 27.75 |
| 25.1642 | 29.90 |
| 25.7310 | 49.96 |
| 27.1482 | 38.49 |
| 27.6772 | 10.70 |
| 27.9857 | 5.32 |
| 29.0996 | 7.66 |
| 29.3985 | 10.88 |
| 29.9267 | 20.17 |
| 30.9874 | 5.22 |
| 31.8056 | 12.06 |
| 32.8799 | 7.23 |
| 33.1991 | 5.73 |
| 34.4861 | 6.97 |
| 36.3854 | 7.95 |
| 36.6246 | 4.89 |
| 37.3258 | 7.90 |
| 37.8748 | 7.87 |
| 38.3143 | 5.55 |
| 40.8261 | 2.60 |
| 42.4567 | 3.57 |
| 43.2056 | 2.48 |
| 43.7464 | 4.48 |
| 45.0366 | 1.28 |
| 46.0177 | 2.48 |
| 48.3545 | 1.47 |

The seed material of crystalline monohydrate of Compound A used in the above procedures was obtained as follows. Approx. 200 mg of Compound A, obtained by a procedure analogous to the synthesis method as described in Example 158 of WO 2018/119036, was added to 400-800 μl of either ethyl acetate or isopropyl acetate and the resulting suspension stirred at 60° C. for 5 days. The precipitate was then filtered and dried under vacuum at 50° C. for 24 hours to yield crystalline monohydrate of Compound A.

### Example 2: Solubility in Polyethylene Glycol

The solubility of an API in polyethylene glycol may be obtained using hot stage microscopy or differential scanning microscopy (DSC). First, the API may be added to molten polyethylene glycol at various concentrations, covering a range below and above the solubility limit of the API in the molten matrix

Hot stage microscopy method: Solidified samples of the API at various concentrations in polyethylene glycol, which have been stored for a period of time at a certain temperature condition, may be heated from room temperature to a temperature above the polyethylene glycol freezing point at different heating rates (e.g. 3° C./min, 10° C./min and 30° C./min). The highest concentration with no visible crystals is considered as the closest approximation of the thermodynamic solubility at a particular storage temperature.

DSC method: Samples of the API at various concentrations in molten polyethylene (above and below the solubility in the matrix) may be poured into DSC pan, in a sample holder together with an empty reference pan), and allowed to solidify. Samples may be measured at different heating rates (e.g. 3° C./min, 5° C./min and 10° C./min), heating from 25° C. to a temperature above the freezing point of the polyethylene glycol. Software may then be used to integrate the DSC curve to obtain the enthalpy change for each sample concentration. Saturation solubility can be obtained from a graph of sample concentration versus enthalpy change and is the point at which enthalpy is lowest.

### Example 3: Crystallisation Inhibition

A 1% solution of PVPVA64 in FaSSIF was prepared by weighing 2.5 g of PVPVA64 in a volumetric flask and dissolving it in 250 mL FaSSIF at 37° C. The complete volume of the volumetric flask was transferred to a suitable Duran bottle. Afterwards, a highly concentrated solution of Compound A monohydrate (obtained according to Example 1) in NMP was prepared (175 mg/mL). At time zero, 679 μL of the highly concentrated Compound A solution (175 mg/mL) was added to the Duran bottle containing 250 mL FaSSIF+1% PVPVA64 (w/v). The added volume Compound A solution was chosen to obtain a theoretical final concentration of 475.5 μg/mL. This concentration results in a suitable degree of supersaturation of Compound A.

TABLE 2
Quantities required to obtain a suitable degree of supersaturation of the
Compound A stock solution in NMP in 250 mL FaSSIF (+1% PVPVA64).

| Degree of | | Cₛₜₐᵣₜ = DS × | | |
|---|---|---|---|---|
| super- | Solubility of | solubility of | | V_{stock solution} |
| saturation | Compound | Compound A in | | transferred to |
| (DS) | A in FaSSIF | FaSSIF | C_{stock solution} | 250 mL FaSSIF |
| — | (μg/mL) | (μg/mL) | (mg/mL) | (μL) |
| 16 | 29.72 | 475.5 | 175.0 | 679 |

A precipitate formed immediately after adding the highly concentrated solution. The mixture was equilibrated at 37° C. for 2 hours while stirring at 300 rpm. After 2 hours, the precipitate was separated from the liquid phase by vacuum filtration. The precipitate was then analyzed with Powder X-Ray Diffraction to check if the API present in the formed precipitate was present as an amorphous or a crystalline form.

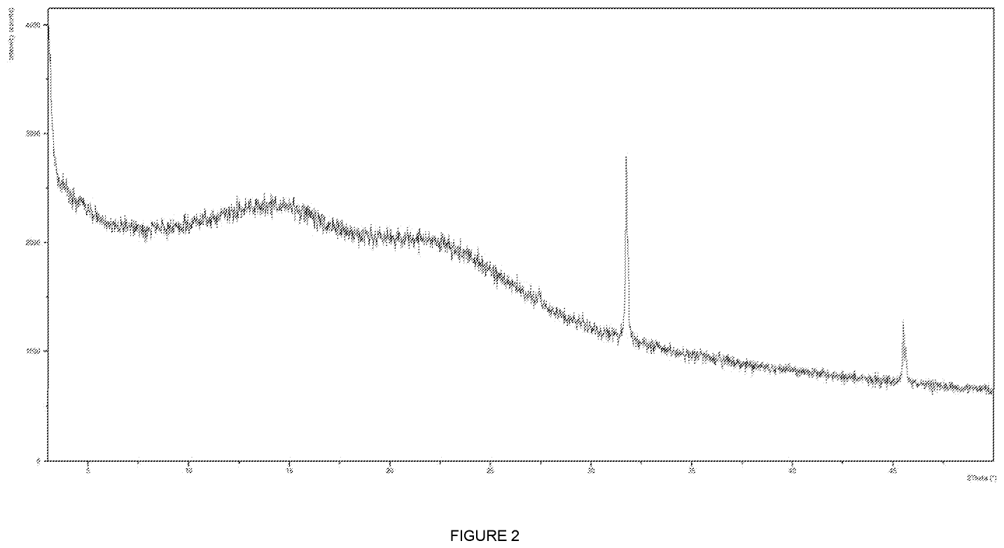

The FaSSIF+1% PVPVA 64 medium resulted in an amorphous precipitate. The XRPD pattern of the precipitate is shown in FIG. 2. The XRPD pattern shows a halo with two peaks superimposed due to NaCl present in the FaSSIF medium.

It was therefore concluded that PVPVA64 functions as crystallisation rate inhibitor. PVPVA64 was also found to be well miscible with PEG1500.

### XRPD Method

X-ray powder diffraction (XRPD) analysis was carried out on a PANalytical (Philips) X'PertPRO MPD diffractometer. The instrument is equipped with a Cu LFF X-ray tube. The compound was spread on a zero-background sample holder

### Instrument Parameters

Generator voltage: 45 kV
Generator amperage: 40 mA

### Geometry: Bragg-Brentano

Stage: spinner stage

### Measurement Conditions

Scan mode: continuous
Scan range: 3 to 50° 2θ
Step size: 0.02°/step
Counting time: 60 sec/step
Spinner revolution time: 1 sec
Radiation type: CuKα

### Incident Beam Path

Program. divergence slit: 15 mm
Soller slit: 0.04 rad
Beam mask: 15 mm
Anti-scatter slit: 1°
Beam knife: +

### Diffracted Beam Path

Long anti scatter shield: +
Soller slit: 0.04 rad
Ni filter: +

### Detector: X'Celerator

### Example 4: Process for Preparing Hard Gelatin Capsules of a Compound A Formulation

Macrogol 1500, all-rac-alpha-Tocopherol (vitE), copovidone (PVPVA64) and Compound A monohydrate (obtained from Example 1) were dispensed, melted and mixed successively into a suitable container at 60° C.±5° C. under nitrogen blanketing until a clear solution was obtained. The obtained bulk solution was transferred into the capsule filling machine hopper pre-heated to 60° C.±5° C. followed by filling into hard gelatin capsules. The filled capsules were collected and stored at room temperature in LDPE bags in suitable containers until packaging in HDPE bottles. 30 capsules were packed in a HDPE bottle followed by induction sealing. After filling the capsules were controlled for appearance and weight. During bottling the number of capsules was counted and after bottling the bottles were checked for seal integrity. Compound A was supplied in the monohydrate form as starting material in an amount equivalent with 2 mg, 10 mg, 50 mg and 200 mg of anhydrous Compound A in the final hard gelatin capsules for oral administration.

Table 3 provides a batch formula for a typical clinical batch size. However, batch sizes can be varied to meet the clinical needs, provided that the drug product quality is not negatively impacted.

TABLE 3
Composition of a 7200 capsule batch size, for each component
the amount used as starting material is indicated

| | Quantity per batch of 7200 capsules | | | |
|---|---|---|---|---|
| | | | 50 mg | 200 mg |
| Component | 2 mg capsule | 10 mg capsule | capsule | capsule |
| Compound A | 14.98 g | 74.88 g | 374.40 g | 1497.60 g |
| monohydrate* | | | | |
| Copovidone | 45.00 g | 72.00 g | 144.00 g | 327.24 g |
| All-rac-alpha | 0.09 g | 0.14 g | 0.29 g | 0.65 g |
| tocopherol (Vit | | | | |
| E) | | | | |
| Macrogol 1500 | 840.24 g | 1293.12 g | 2361.60 g | 4719.60 g |
| Nitrogen^{†} | q.s. | q.s. | q.s. | q.s. |
| White opaque | 7200 size 4 | 7200 size 3 | 7200 size 1 | 7200 size 00 |
| hard gelatin | capsules | capsules | capsules | capsules |
| capsule^{†} | | | | |

*Compound A is used in the monohydrate form as starting material. The amount of Compound A monohydrate is calculated based on the active anhydrous equivalent in the final formulation, where a conversion factor from the anhydrous form to the monohydrate is 1.04.
^{†}Inert processing aid. q.s. = quantum sufficit.
^{†}the empty hard gelatin capsule is made up of gelatin and titanium dioxide.

Table 4 provides the components used to prepare each of the different capsule sizes.

TABLE 4
Components used to prepare the Compound A-eq. hard gelatin capsules

| | 2 mg capsule | | | 10 mg capsule | | | 50 mg capsule | | | 200 mg capsule | | |
|---|---|---|---|---|---|---|---|---|---|---|---|---|
| Component | Quantity | | w/w % | Quantity | | w/w % | Quantity | | w/w % | Quantity | | w/w % |
| Compound A | 2.08 | mg | 1.66 | 10.4 | mg | 5.20 | 52 | mg | 13.00 | 208 | mg | 22.88 |
| monohydrate* | | | | | | | | | | | | |
| Copovidone | 6.25 | mg | 5.00 | 10 | mg | 5.00 | 20 | mg | 5.00 | 45.45 | mg | 5.00 |
| All-rac-alpha | 12.5 | μg | 0.01 | 20 | μg | 0.01 | 40 | μg | 0.01 | 90.9 | μg | 0.01 |
| tocopherol^{‡} | | | | | | | | | | | | |
| Macrogol | 116.7 | mg | 93.33 | 179.6 | mg | 89.79 | 328 | mg | 81.99 | 655.5 | mg | 72.11 |

| 1500 | | | | | | | | |
|---|---|---|---|---|---|---|---|---|
| White opaque | 1 size | n/a | 1 size | n/a | 1 size | n/a | 1 size | n/a |
| hard gelatin | 4 capsule | | 3 capsule | | 1 capsule | | 00 capsule | |
| capsule^{†} | | | | | | | | |

*Compound A is used in the monohydrate form as starting material. The amount of Compound A monohydrate is calculated based on the active anhydrous equivalent in the 10 final formulation (2 mg, 10 mg, 50 mg, 200 mg), where a conversion factor from the anhydrous form to the monohydrate is 1.04. ^{‡}vitamin E. ^{†}the empty hard gelatin capsule is made up of gelatin and titanium dioxide. n/a means not available.

### Example 5: Non-Clinical Study

A pharmacokinetic (PK) study was carried out using a formulation of Compound A in PEG1500/PVPVA64.

Compound A was formulated in 10 mg (see Table 4) capsules for assessment in a fasted dog PK study. A single dose was administered to fasted male beagle dogs (N=4/group) in a crossover manner with at least a 12-day washout period between doses.

TABLE 5
Mean (SD) Plasma PK Parameters of Compound A in Dog
Following a Single 10 mg Dose Administered as a capsule

| | | Cₘₐₓ | | Tₘₐₓ | | AUC₀₋₂₄ₕ | | AUC₀₋₉₆ₕ | | AUC_{inf} | | t_{1/2} | | Fᵣₑₗ^{a} | |
|---|---|---|---|---|---|---|---|---|---|---|---|---|---|---|---|
| | N | (ng/mL) | | (h) | | (ng h/mL) | | (ng h/mL) | | (ng h/mL) | | (h) | | (%) | |
| Formulation 1- | 4 | 624 | (83.4) | 3.5 | (1.0) | 7410 | (949) | 11200 | (1770) | 11300 | (1830) | 15 | (1.1) | — | |
| Solution (90% | | | | | | | | | | | | | | | |
| PEG400: | | | | | | | | | | | | | | | |
| 10% PVPVA64) | | | | | | | | | | | | | | | |
| PEG1500/ | 3 | 978 | (178) | 1.7 | (0.58) | 10700 | (2050) | 15300 | (2620) | 15900 | (2550) | 24 | (3.7) | 131 | (17) |
| PVPVA64/ | | | | | | | | | | | | | | | |

Vit E

^{a}Relative bioavailability calculated using AUC_{inf} Solution (Formulation 1) was used as the reference.
AUC = area under the plasma concentration-time curve; AUC₀₋₂₄ₕ = AUC from time 0 to 24 hours postdose; AUC₀₋₉₆ₕ = AUC from time 0 to 96 hours postdose; AUC_{inf} = AUC from time 0 to infinity with extrapolation of the terminal phase; Cₘₐₓ = maximum observed plasma concentration; Fᵣₑₗ = relative bioavailability; N = number of animals; SD = standarddeviation; t_{1/2} = half-life; Tₘₐₓ = time correspondent to the maximum observed plasma concentration; Vit E = Vitamin E.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention and that embodiments within the scope of these claims and their equivalents be covered thereby.

1. A pharmaceutical formulation, comprising:
a) a polyethylene glycol having a freezing point of at least about 30° C.;
b) an active pharmaceutical ingredient that is soluble in molten polyethylene glycol wherein the polyethylene glycol is as defined in a); and
c) a crystallisation rate inhibitor.

2. The pharmaceutical formulation of claim 1, wherein the pharmaceutical formulation is a solid formulation.

3. The pharmaceutical formulation of claim 1, wherein the polyethylene glycol has a freezing point of from about 35° C. to about 70° C.

4. The pharmaceutical formulation of claim 3, wherein the polyethylene glycol has an average molecular weight of at least about 900 g/mol or at least about 1000 g/mol.

5. The pharmaceutical formulation of claim 4, wherein the polyethylene glycol has an average molecular weight of from about 1000 to about 20000 g/mol.

6. The pharmaceutical formulation of claim 5, wherein the polyethylene glycol is PEG1500, PEG2000, PEG3000, PEG3350 or PEG4000.

7. The pharmaceutical formulation of claim 1, wherein the crystallisation rate inhibitor is selected from polyvinylpyrrolidone (PVP), a polyvinylpyrrolidone-vinyl acetate copolymer (PVPVA), a poly(meth)acrylate polymer, a cyclodextrin and a derivative thereof, hydroxypropylcellulose, methylcellulose, hydroxypropyl methylcellulose (HPMC), hydroxypropyl methylcellulose acetate succinate (HPMCAS), a poloxamer, and combinations thereof.

8. The pharmaceutical formulation of claim 7, wherein the crystallisation rate inhibitor is selected from polyvinylpyrrolidone (PVP) and a polyvinylpyrrolidone-vinyl acetate copolymer (PVPVA), and a combination thereof.

9. The pharmaceutical formulation of claim 8, wherein the crystallisation rate inhibitor is polyvinylpyrrolidone-vinyl acetate copolymer (PVPVA).

10. The pharmaceutical formulation of claim 9, wherein the PVPVA is a copolymer of 1-vinyl-2-pyrrolidone and vinyl acetate in a ratio of 6:4 by mass (PVPVA64).

11. The pharmaceutical formulation of claim 1, wherein the formulation comprises from about 1 w/w % to about 15 w/w % of the crystallisation rate inhibitor relative to the total weight of the formulation.

12. The pharmaceutical formulation of claim 11, wherein the formulation comprises about 1 w/w %, about 5 w/w % or about 10 w/w % of the crystallisation rate inhibitor.

13. The pharmaceutical formulation of claim 1, wherein the formulation further comprises an antioxidant.

14. The pharmaceutical formulation of claim 13, wherein the antioxidant is selected from tocopherol (vitamin E), thiodipropionic acid, lipoic acid, hydroquinone, phytic acid, monothioglycerol, sodium thioglycolate, thioglycol, beta carotene, butylated hydroxyanisole (BHA), butylated hydroxytoluene (BHT), cysteine, cysteine hydrochloride, propyl gallate (PG), sodium metabisulfite, ascorbyl palmitate, ascorbyl stearate, potassium metabisulfite, disodium EDTA (ethylenediamine tetraacetic acid; also known as disodium edentate), EDTA, erythorbic acid, ethoxyquin, glutathione, gum guaiac, lecithin, propyl gallate, TBHQ (tert butyl hydroxyquinone), tartaric acid, citric acid, citric acid monohydrate, methane sulfonic acid, methionine, sodium metabisulfite, sodium thiosulfate, sodium sulphite, and combinations thereof.

15. The pharmaceutical formulation of claim 14, wherein the antioxidant is selected from tocopherol (vitamin E), lipoic acid, hydroquinone, monothioglycerol, thioglycol, beta carotene, butylated hydroxyanisole (BHA), butylated hydroxytoluene (BHT), propyl gallate (PG), ascorbyl palmitate, ascorbyl stearate, ethoxyquin, propyl gallate, TBHQ (tert butyl hydroxyquinone), and combinations thereof.

16. The pharmaceutical formulation of claim 15, wherein the antioxidant is all-rac-alpha tocopherol.

17. The pharmaceutical formulation of claim 1, wherein the formulation comprises from about 0.1 w/w % to about 40 w/w %, from about 1 w/w % to about 30 w/w %, or from about 5 w/w % to about 25 w/w % of the active pharmaceutical ingredient relative to the total weight of the formulation.

18. The pharmaceutical formulation of claim 17, wherein the formulation comprises from about 20 w/w % to about 25 w/w % of the active pharmaceutical ingredient relative to the total weight of the formulation.

19. The pharmaceutical formulation of claim 1, wherein the active pharmaceutical ingredient is a MALT1 inhibitor.

20. The pharmaceutical formulation of claim 1, wherein the active pharmaceutical ingredient is a compound of Formula (I) 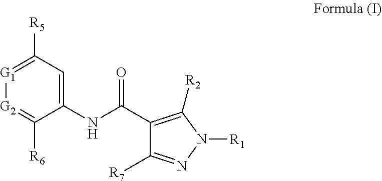
wherein
R₁ is selected from the group consisting of
i) naphthalen-1-yl, optionally substituted with a fluoro or amino substituent; and
ii) a heteroaryl of nine to ten members containing one to four heteroatoms selected from the group consisting of O, N, and S; such that no more than one heteroatom is O or S; wherein said heteroaryl of ii) is optionally independently substituted with one or two substituents selected from deuterium, methyl, ethyl, propyl, isopropyl, trifluoromethyl, cyclopropyl, methoxymethyl, difluoromethyl, 1,1-difluoroethyl, hydroxymethyl, 1-hydroxyethyl, 1-ethoxyethyl, hydroxy, methoxy, ethoxy, fluoro, chloro, bromo, methylthio, cyano, amino, methylamino, dimethylamino, 4-oxotetrahydrofuran-2-yl, 5-oxopyrrolidin-2-yl, 1,4-dioxanyl, aminocarbonyl, methylcarbonyl, methylaminocarbonyl, oxo, 1-(t-butoxycarbonyl)azetidin-2-yl, N-(methyl)formamidomethyl, tetrahydrofuran-2-yl, 3-hydroxy-pyrrolidin-1-yl, pyrrolidin-2-yl, 3-hydroxyazetidinyl, azetidin-3-yl, or azetidin-2-yl;
R₂ is selected from the group consisting of C₁₋₄alkyl, 1-methoxy-ethyl, difluoromethyl, fluoro, chloro, bromo, cyano, and trifluoromethyl;
G₁ is N or C(R₄);
G₂ is N or C(R₃); such that only one of G₁ and G₂ are N in any instance;
R₃ is independently selected from the group consisting of trifluoromethyl, cyano, C₁₋₄alkyl, fluoro, chloro, bromo, methylcarbonyl, methylthio, methylsulfinyl, and methanesulfonyl; or, when G₁ is N, R₃ is further selected from C₁₋₄alkoxycarbonyl;
R₄ is selected from the group consisting of
i) hydrogen, when G₂ is N;
ii) C₁₋₄alkoxy;
iii) cyano;
iv) cyclopropyloxy;
v) a heteroaryl selected from the group consisting of triazolyl, oxazolyl, isoxazolyl, pyrazolyl, pyrrolyl, thiazolyl, tetrazolyl, oxadiazolyl, imidazolyl, 2-amino-pyrimidin-4-yl, 2H-[1,2,3]triazolo[4,5-c]pyridin-2-yl, 2H-[1,2,3]triazolo[4,5-b]pyridin-2-yl, 3H-[1,2,3]triazolo[4,5-b]pyridin-3-yl, 1H-[1,2,3]triazolo[4,5-c]pyridin-1-yl, wherein the heteroaryl is optionally substituted with one or two substituents independently selected from oxo, C₁₋₄alkyl, carboxy, methoxycarbonyl, aminocarbonyl, hydroxymethyl, aminomethyl, (dimethylamino)methyl, amino, methoxymethyl, trifluoromethyl, amino(C₂₋₄alkyl)amino, or cyano;
vi) 1-methyl-piperidin-4-yloxy;
vii) 4-methyl-piperazin-1-ylcarbonyl;
viii) (4-aminobutyl)aminocarbonyl;
ix) (4-amino)butoxy;
x) 4-(4-aminobutyl)-piperazin-1-ylcarbonyl;
xi) methoxycarbonyl;
xii) 5-chloro-6-(methoxycarbonyl)pyridin-3-ylaminocarbonyl;
xiii) 1,1-dioxo-isothiazolidin-2-yl;
xiv) 3-methyl-2-oxo-2,3-dihydro-1H-imidazol-1-yl;
xv) 2-oxopyrrolidin-1-yl;
xvi) (E)-(4-aminobut-1-en-1-yl-aminocarbonyl;
xvii) difluoromethoxy; and
xviii) morpholin-4-ylcarbonyl;
R₅ is independently selected from the group consisting of hydrogen, chloro, fluoro, bromo, methoxy, methylsulfonyl, cyano, C₁₋₄alkyl, ethynyl, morpholin-4-yl, trifluoromethyl, hydroxyethyl, methylcarbonyl, methylsulfinyl, 3-hydroxy-pyrrolidin-1-yl, pyrrolidin-2-yl, 3-hydroxyazetidinyl, azetidin-3-yl, azetidin-2-yl, methylthio, and 1,1-difluoroethyl;
or R₄ and R₅ may be taken together to form 8-chloro-4-methyl-3-oxo-3,4-dihydro-2H-benzo[b][1,4]oxazin-6-yl, 8-chloro-3-oxo-3,4-dihydro-2H-benzo[b][1,4]oxazin-6-yl, 2-methyl-1-oxo-1,2,3,4-tetrahydroisoquinolin-7-yl, 4-methyl-3-oxo-3,4-dihydro-2H-benzo[b][1,4]oxazin-6-yl, 3-oxo-3,4-dihydro-2H-benzo[b][1,4]oxazin-6-yl, 1-methyl-1H-pyrazolo[3,4-b]pyridin-5-yl, 1H-pyrazolo[3,4-b]pyridin-5-yl, 2,3-dihydro-[1,4]dioxino[2,3-b]pyridin-5-yl, 1,3-dioxolo[4,5]pyridine-5-yl, 1-oxo-1,3-dihydroisobenzofuran-5-yl, 2,2-dimethylbenzo[d][1,3]dioxol-5-yl, 2,3-dihydrobenzo[b][1,4]dioxin-6-yl, 1-oxoisoindolin-5-yl, or 2-methyl-1-oxoisoindolin-5-yl, 1H-indazol-5-yl;
R₆ is hydrogen, C₁₋₄alkyl, fluoro, 2-methoxy-ethoxy, chloro, cyano, or trifluoromethyl; and
R₇ is hydrogen or fluoro;
or an enantiomer, diastereomer, solvate, or pharmaceutically acceptable salt form thereof.

21. The pharmaceutical formulation according to claim 20, wherein the compound of Formula (I) is 1-(1-oxo-1,2-dihydroisoquinolin-5-yl)-5-(trifluoromethyl)-N-(2-(trifluoromethyl)pyridin-4-yl)-1H-pyrazole-4-carboxamide: 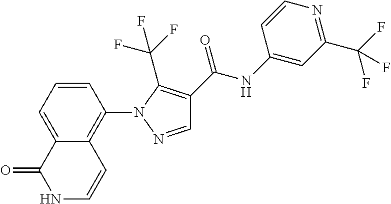 [SMILES: O=C(Nc1ccnc(C(F)(F)F)c1)c1cnn(-c2cccc3c(=O)[nH]ccc23)c1C(F)(F)F]
or a solvate or pharmaceutically acceptable salt thereof.

22. A solid dosage form comprising the pharmaceutical formulation of claim 1.

23. The solid dosage form of claim 22, wherein the dosage form is an oral dosage form.

24. The solid dosage form of claim 22, wherein the dosage form comprises a capsule encapsulating the pharmaceutical formulation.

25. The solid dosage form of claim 24, wherein the capsule is a hard gelatin capsule.

26. The solid dosage form of claim 22, wherein the formulation comprises about 2 to about 1000 mg of the active pharmaceutical ingredient.

27. The solid dosage form of claim 26, wherein the formulation comprises 2, 10, 50 or 200 mg of the active ingredient 1-(1-oxo-1,2-dihydroisoquinolin-5-yl)-5-(trifluoromethyl)-N-(2-(trifluoromethyl)pyridin-4-yl)-1H-pyrazole-4-carboxamide:

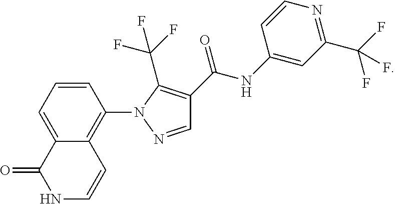 [SMILES: O=C(Nc1ccnc(C(F)(F)F)c1)c1cnn(-c2cccc3c(=O)[nH]ccc23)c1C(F)(F)F]

28. A method of treating a disease, syndrome, condition, or disorder, wherein said disease, syndrome, condition, or disorder is affected by the inhibition of MALT1, comprising administering to a subject in need thereof a therapeutically effective amount of a pharmaceutical formulation of claim 20.

29. The method of claim 28 wherein said disease, syndrome, condition, or disorder is selected from cancer and immunological diseases.

30-31. (canceled)

32. A process for preparing a pharmaceutical formulation according to claim 1, comprising the steps of:
a) forming a melt comprising polyethylene glycol having a freezing point of at least about 30° C., an active pharmaceutical ingredient, and a crystallisation rate inhibitor, wherein the forming a melt step comprises heating polyethylene glycol to a temperature above its freezing point; and
b) cooling the melt below the freezing point of the polyethylene glycol;
to provide a pharmaceutical formulation according to claim 1.

33. A process for preparing a solid dosage form according to claim 24, the process comprising the steps of:
a) forming a melt comprising polyethylene glycol having a freezing point of at least about 30° C., an active pharmaceutical ingredient, and a crystallisation rate inhibitor, wherein the forming a melt step comprises heating polyethylene glycol to a temperature above its freezing point;
b) filling a hard capsule with the melt; and
c) cooling the filled capsule below the freezing point of the polyethylene glycol;
to provide a solid dosage form according to claim 24.

34. The process of claim 32, wherein the polyethylene glycol is heated to a temperature of at least about 5° C. above its freezing point.

35. The process of claim 32, wherein the polyethylene glycol is heated to a temperature of at least about 10° C. above its freezing point.

36. The process of claim 32, wherein the active pharmaceutical ingredient used as starting material in step b) of the process is a crystalline form of Compound A monohydrate producing an X-ray powder diffraction pattern comprising peaks at 16.4, 23.7 and 25.7 degrees two theta±0.2 degrees two theta.

37. A solid dosage form comprising
a) a polyethylene glycol having a freezing point of at least about 30° C.; and
b) an active pharmaceutical ingredient that is soluble in molten polyethylene glycol wherein the polyethylene glycol is as defined in a);
wherein the solid dosage form is a HPMC capsule.

38. The process of claim 33, wherein the polyethylene glycol is heated to a temperature of at least about 5° C. above its freezing point.

39. The process of claim 33, wherein the polyethylene glycol is heated to a temperature of at least about 10° C. above its freezing point.

40. The process of claim 33, wherein the active pharmaceutical ingredient used as starting material in step b) of the process is a crystalline form of Compound A monohydrate producing an X-ray powder diffraction pattern comprising peaks at 16.4, 23.7 and 25.7 degrees two theta±0.2 degrees two theta.
